(19) United States
(12) Patent Application Publication
(10) Pub. No.: US 2009/0005355 A1
(43) Pub. Date: Jan. 1, 2009

(54) Piperidine Compound and Process for Preparing the Same

(75) Inventors: Tsutomu Miyake, Osaka (JP); Takeshi Yamanaka, Osaka (JP); Hidetoshi Asai, Osaka (JP); Yoshihiro Terakawa, Osaka (JP)

Correspondence Address: BROWDY AND NEIMARK, P.L.L.C.;624 NINTH STREET, NW, SUITE 300, WASHINGTON, DC, 20001-5303, US

(73) Assignee: Tanabe Seiyaku Co., Ltd., Osaka-shi (JP)

(21) Appl. No.: 11/631,282

(22) PCT Filed: Jul. 1, 2005
(86) PCT No.: PCT/JP05/12630

§ 371 Date: Jan. 3, 2007

(30) Foreign Application Priority Data
Jul. 2, 2004 (JP) 2004-196360

(51) Int. Cl. A61K 31/445; C07D 211/40; C07D 401/04; C07D 401/14; C07D 417/06; A61P 13/00; A61K 31/397; A61K 31/4545; A61K 31/496; A61K 31/541

(52) U.S. Cl. 514/210.18; 546/226; 546/189; 544/364; 544/60; 544/58.2; 514/330; 514/316; 514/253.13; 514/227.8

(57) ABSTRACT

The present invention is to provide a piperidine compound represented by the formula [I]:

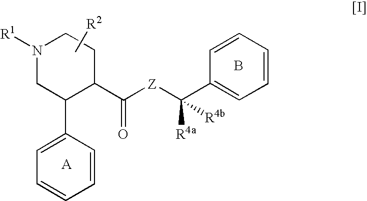

wherein
Ring A is an optionally substituted benzene ring,
Ring B is an optionally substituted benzene ring,
R¹ is hydrogen atom or a substituent for amino group,
R² is hydrogen atom, an optionally substituted hydroxyl group, an optionally substituted amino group, an optionally substituted alkyl group, a substituted carbonyl group or a halogen atom,
Z is oxygen atom or —N(R³)—,
R³ is hydrogen atom or an optionally substituted alkyl group,
R^{4a} and R^{4b} may be the same or different, and each is hydrogen atom or an optionally substituted alkyl group, or may be bonded to each other at the both ends to form an alkylene group,
   or a pharmaceutically acceptable salt thereof, which has an excellent tachykinin receptor antagonistic action.

### TECHNICAL FIELD

The present invention relates to a piperidine compound having an excellent activity of tachykinin receptor antagonist, and a process for preparing the piperidine compound.

### BACKGROUND ART

Tachykinin is a general name for a group of neuropeptides, and there have been known substance P (hereinafter referred to as “SP”), neurokinin-A, and neurokinin-B in mammals. These peptides are known to exhibit various kinds of biological activities by binding their corresponding receptors which exist in vivo (neurokinin-1, neurokinin-2, neurokinin-3). Among them, SP is one of those which have been studied the longest and in detail. Its existence was confirmed in an extract of horse intestinal tube in 1931, and it was a peptide comprising 11 amino acids, whose structure was determined in 1971.

SP exists widely in central and peripheral nervous systems, and it has physiological activities such as vasodilative action, vascular permeability promoting action, smooth muscle contracting action, neuronal excitatory action, salivary action, diuretic action, immunological action, etc., as well as a function of neurotransmitter of the primary sensory neuron. Especially, it is known that SP released from the terminal of posterior horn of spinal cord upon pain impulse transfers pain information to the secondary sensory neuron, and that SP released from the peripheral terminus induces an inflammatory response via its receptors. From these facts, SP is considered to be involved in various diseases (for example, pain, inflammation, allergy, pollakiuria, urinary incontinence, respiratory disease, mental disorder, depression, anxiety, emesis, etc.), and also, SP is considered to be involved in Alzheimer-type dementia [Review: Physiological Reviews, vol. 73, pp. 229-308 (1993), Journal of Autonomic Pharmacology, vol. 13, pp. 23-93 (1993)].

[Non-Patent publication 1] Physiological Reviews, vol. 73, pp. 229-308 (1993)
[Non-Patent publication 2] Journal of Autonomic Pharmacology, vol. 13, pp. 23-93 (1993)

### SUMMARY OF THE INVENTION

Currently, as a therapeutic agent for the above-mentioned various diseases (especially for emesis, depression, urinary disorder, etc.), there have not been discovered yet any compound having an excellent tachykinin receptor antagonistic action (specifically, SP receptor antagonistic action), and having sufficiently satisfying safety and sustainability (metabolism, dynamics in vivo, and absorption), etc. Therefore, a compound has been sought for which has an excellent tachykinin receptor antagonistic action, and has sufficiently satisfying clinical effect as the therapeutic agent.

Accordingly, an object of the present invention is to provide a compound having excellent tachykinin receptor antagonistic action, and having a clinical satisfying effect in terms of safety, sustainability (metabolism, dynamics in vivo and absorption), etc.

The present invention relates to a piperidine compound represented by the formula [I]:

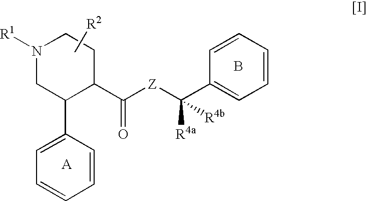

wherein
Ring A represents an optionally substituted benzene ring,
Ring B represents an optionally substituted benzene ring,
R¹ represents hydrogen atom or a substituent for amino group,
R² represents hydrogen atom, an optionally substituted hydroxyl group, an optionally substituted amino group, an optionally substituted alkyl group, a substituted carbonyl group or a halogen atom,
Z represents oxygen atom or a group represented by the formula: —N(R³)—,
R³ represents hydrogen atom or an optionally substituted alkyl group,
R^{4a} and R^{4b} are the same or different from each other and each is hydrogen atom or an optionally substituted alkyl group, or may be bonded to each other at the both ends to form an alkylene group,
   or a pharmaceutically acceptable salt thereof.

### BEST MODE FOR CARRYING OUT THE INVENTION

In the present invention, Ring A represents an optionally substituted benzene ring, and a substituent of the benzene ring is exemplified by an optionally substituted alkyl group, a halogen atom, cyano group, hydroxyl group which may be protected or an alkoxy group. Ring A may have 1 to 3 of these substituent(s) which are the same or different.

In the present invention, Ring B represents an optionally substituted benzene ring, and a substituent of the benzene ring is exemplified by a haloalkyl group, a halogen atom, cyano group, phenyl group, a heterocyclic group having 1 to 4 atoms selected from nitrogen atom, oxygen atom and sulfur atom as hetero atom(s), an alkyl group, hydroxyl group which may be protected or an alkoxy group. Ring B may have 1 to 3 of these substituent(s) which are the same or different.

A preferred example of Ring A and Ring B in the compound of the present invention is exemplified by a compound wherein Ring A is a benzene ring of the formula:

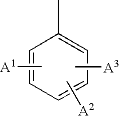

and Ring B is a benzene ring of the formula:

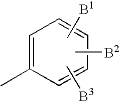

wherein A¹, A² and A³ are the same or different, and each is hydrogen atom, a halogen atom, an optionally substituted alkyl group, hydroxyl group which may be protected or an alkoxy group, B¹, B² and B³ are the same or different, and each is hydrogen atom, a haloalkyl group, a halogen atom, cyano group, phenyl group, a heterocyclic group having 1 to 4 atoms selected from nitrogen atom, oxygen atom and sulfur atom as hetero atom(s), an alkyl group, hydroxyl group which may be protected or an alkoxy group. The substituent for the optionally substituted alkyl group is exemplified by a halogen atom, etc. The haloalkyl group is exemplified by an alkyl group substituted by 1 to 3 halogen atoms which may be the same or different from each other, and specifically mentioned a trihalogenoalkyl group. The trihalogenoalkyl group is exemplified by trifluoromethyl group or trichloromethyl group, etc. The heterocyclic group having 1 to 4 atoms selected from nitrogen atom, oxygen atom and sulfur atom as hetero atom(s) is exemplified by tetrazolyl group.

In the present invention, the protective group for the optionally protected hydroxyl group is exemplified by a conventionally used protective group such as an optionally substituted arylalkyl group, an optionally substituted silyl group, an acyl group, etc. Of these, preferred is exemplified by an arylalkyl group such as benzyl group, phenethyl group, etc., a substituted silyl group such as tert-butyldimethylsilyl group, tert-butyldiphenylsilyl group, etc., an acyl group such as formyl group, acetyl group, propionyl group, malonyl group, acryloyl group, benzoyl group, etc.

In the present invention, R¹ represents hydrogen atom or a substituent for amino group, and the substituent of the amino group in R¹ is exemplified by an optionally substituted alkyl group, an optionally substituted cycloalkyl group, an optionally substituted aryl group, an optionally substituted amino group, an optionally substituted hydroxyl group, a substituted carbonyl group, a substituted sulfinyl group, a substituted sulfonyl group or an optionally substituted heterocyclic group.

Of these, R¹ is preferably an optionally substituted alkyl group, an optionally substituted cycloalkyl group, a substituted carbonyl group, a substituted sulfonyl group or an optionally substituted heterocyclic group, and R¹ is further preferable a substituted carbonyl group, a substituted sulfonyl group or an optionally substituted heterocyclic group.

In the present invention, the substituent of the optionally substituted alkyl group of R¹ is exemplified by an alkoxycarbonyl group, morpholinocarbonyl group, a dialkylaminocarbonyl group wherein the alkyl moiety thereof is optionally substituted by hydroxyl group, an optionally substituted heterocyclic group, hydroxyl group, hydroxyalkylaminocarbonyloxy group, an alkylpiperazinocarbonyl group, an alkanoyl group, an alkylsulfonyl group, pyrrolidinylsulfonyl group, cyano group, carboxyl group, a halogen atom, an alkylthio group or an alkanoylamino group. The alkyl group may have 1 to 3 substituent(s). Preferred substituent of the heterocyclic group of which is substituted by the alkyl group, is exemplified by an alkanoyl group optionally substituted by hydroxyl group, an alkyl group or oxo group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, oxadiazolyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

In the present invention, the substituent of the optionally substituted cycloalkyl group of R¹ is exemplified by hydroxyl group, an alkylenedioxy group or oxo group.

In the present invention, the substituent of the optionally substituted aryl group of R¹ is exemplified by hydroxyl group, an alkyl group, cyano group, a halogen atom, etc. The aryl group is exemplified by phenyl group, naphthyl group, anthracenyl group or phenanthrenyl group.

In the present invention, the substituent of the optionally substituted amino group of R¹ is exemplified by

(1) an optionally substituted alkyl group,
(2) an optionally substituted cycloalkyl group,
(3) an optionally substituted aryl group or
(4) a heterocyclic group having 1 to 4 atoms selected from nitrogen atom, oxygen atom and sulfur atom as hetero atom(s), and the heterocyclic group may have a substituent(s).

The substituent of the optionally substituted alkyl group in the above-mentioned (1) is exemplified by a dialkylaminocarbonyl group, an alkoxy group, a dialkylamino group, cyano group, morpholino group, pyridyl group or a halogen atom.

The substituent of the substituted cycloalkyl group of the above-mentioned (2) is exemplified by hydroxyl group, an alkyl group, cyano group, a halogen atom, etc.

The substituent of the optionally substituted aryl group of substituent the above-mentioned (3) is exemplified by hydroxyl group, an alkyl group, cyano group, a halogen atom, etc. The aryl group is exemplified by phenyl group, naphthyl group, anthracenyl group or phenanthrenyl group.

The heterocyclic group having 1 to 4 atoms selected from nitrogen atom, oxygen atom and sulfur atom as hetero atom(s) of the above-mentioned (4) is exemplified by a saturated or unsaturated monocyclic or bicyclic heteroaromatic group, and may include, for example, thienyl group, furyl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. Of these heterocyclic groups, suitably used are pyridyl group, pyrrolyl group, piperazinyl group, quinolyl group, piperidinyl group, pyrimidinyl group, thiazolyl group, pyrazinyl group, morpholino group, indolyl group, cinnolinyl group, furyl group, thienyl group, pyrrolidinyl group, imidazolidinyl group, etc. The substituent of the heterocyclic group is exemplified by a dialkylamino group, an alkoxycarbonyl group, an alkyl group, an alkoxy group, oxo group, hydroxyl group or a halogen atom.

In the present invention, the substituent of the optionally substituted hydroxyl group of R¹ is exemplified by an optionally substituted alkyl group. The substituent of the optionally substituted alkyl group is exemplified by an optionally substituted hydroxyl group, a dialkylamino group or a heteromonocyclic group having 1 to 4 atom(s) selected from sulfur atom, nitrogen atom and oxygen atom as hetero atom(s) (the heteromonocyclic group may have a substituent(s).). The substituent of the optionally substituted hydroxyl group is exemplified by an alkyl group, an alkylsulfonyl group or tetrahydropyranyl group. The heteromonocyclic group is exemplified by pyridyl group, piperidinyl group, morpholino group, isoxazolyl group, triazolyl group, tetrazolyl group, pyrrolidinyl group, or imidazolidinyl group. The substituent of the monocyclic heterocyclic group is exemplified by an alkyl group and phenyl group.

In the present invention, the substituent of the substituted carbonyl group of R¹ is exemplified by

(1) an optionally substituted alkyl group,
(2) an optionally substituted cycloalkyl group,
(3) an optionally substituted aryl group,
(4) an optionally substituted heterocyclic group,
(5) an optionally substituted amino group
(6) an optionally substituted alkoxy group or
(7) an optionally substituted hydroxyl group.

The substituent of the optionally substituted alkyl group of the above-mentioned (1) is exemplified by

(I) hydroxyl group,
(II) a substituted carbonylamino group,
(III) an optionally substituted aminocarbonyl group,
(IV) an alkanoyl group,
(V) an alkylsulfonyl group,
(VI) an optionally substituted heterocyclic group or
(VII) amino group.

The substituent of the substituted carbonylamino group of the above-mentioned (II) is exemplified by (i) hydroxyl group, (ii) an optionally substituted alkyl group or (iii) an optionally substituted heterocyclic group, etc. The substituent of the optionally substituted alkyl group of the above-mentioned (ii) is exemplified by hydroxyl group or a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The substituent of the optionally substituted heterocyclic group of the above-mentioned (iii) is exemplified by an alkanoyl group optionally substituted by hydroxyl group, oxo group or hydroxyl group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted aminocarbonyl group of the above-mentioned (III) is exemplified by (i) an optionally substituted alkyl group or (ii) an optionally substituted heterocyclic group. The substituent of the optionally substituted alkyl group of the above-mentioned (i) is exemplified by hydroxyl group or a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The substituent of the optionally substituted heterocyclic group of the above-mentioned (ii) is exemplified by an alkanoyl group optionally substituted by hydroxyl group, oxo group or hydroxyl group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted heterocyclic group of the above-mentioned (VI) is exemplified by oxo group or an alkyl group. The heterocyclic group may have 1 or 2 substituent(s). The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted cycloalkyl group of the above-mentioned (2) is exemplified by hydroxyl group, an alkyl group, oxo group, an alkoxycarbonyl group, oxopyrrolidinyl group, cyano group, a halogen atom, etc. The cycloalkyl group may have 1 or 2 substituent(s).

The substituent of the optionally substituted aryl group of the above-mentioned (3) is exemplified by hydroxyl group, an alkyl group, cyano group, a halogen atom, etc. The aryl group is exemplified by phenyl group, naphthyl group, anthracenyl group or phenanthrenyl group.

The substituent of the optionally substituted heterocyclic group of the above-mentioned (4) is exemplified by

(I) oxo group,
(II) an optionally substituted alkanoyl group,
(III) an optionally substituted alkyl group,
(IV) hydroxyl group,
(V) an alkoxycarbonyl group,
(VI) an alkylsulfonyl group,
(VII) pyrimidinyl group,
(VIII) cyano group or
(IX) a dialkylaminocarbonyl group.

The heterocyclic group may have 1 to 3 substituent(s). The heterocyclic group is exemplified by a heteromonocyclic group having 1 to 4 atoms selected from sulfur atom, nitrogen atom and oxygen atom as hetero atom(s). The heteromonocyclic group is exemplified by piperidinyl group, piperazinyl group, pyridyl group, tetrazolidyl group, pyrrolidinyl group, imidazolidinyl group, morpholinyl group, thiomorpholinyl group, tetrahydropyranyl group, tetrahydrothiopyranyl group or azetidinyl group.

The substituent of the optionally substituted alkanoyl group of the above-mentioned (II) is exemplified by hydroxyl group, etc.

The substituent of the optionally substituted alkyl group of the above-mentioned (III) is exemplified by a halogen atom, hydroxyl group or a heterocyclic group having 1 to 4 atoms selected from nitrogen atom, oxygen atom and sulfur atom as hetero atom(s), and the heterocyclic group may have a substituent(s). The alkyl group may have 1 to 3 substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted amino group of the above-mentioned (5) is exemplified by an alkyl group optionally substituted by hydroxyl group, a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The amino group may have 1 to 2 substituent(s).

The substituent of the optionally substituted alkoxy group of the above-mentioned (6) is exemplified by hydroxyl group.

The optionally substituted hydroxyl group of the above-mentioned (7) is exemplified by a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidyl group, piperazinyl group, morpholinyl group, thiomorpholinyl group, tetrahydropyranyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

In the present invention, the substituent of the substituted sulfinyl group of R¹ is hydroxyl group or an optionally substituted alkyl group. The substituent of the optionally substituted alkyl group is hydroxyl group.

In the present invention, the substituent of the substituted sulfonyl group of R¹ is an optionally substituted alkyl group. The substituent of the optionally substituted alkyl group is hydroxyl group or an alkanoyloxy group.

In the present invention, the substituent of the optionally substituted heterocyclic group of R¹ is an optionally substituted alkanoyl group, an alkoxycarbonyl group, a substituted cycloalkyl group, an alkylsulfonyl group, an optionally substituted alkyl group, a dialkylaminocarbonyl group, hydroxyl group, oxo group or a substituted pyridyl group. The substituent(s) of optionally substituted alkanoyl group is exemplified by hydroxyl group. The substituent(s) of substituted cycloalkyl group is exemplified by hydroxyl group. The substituent(s) of optionally substituted alkyl group is exemplified by halogen atom(s). The substituent(s) of substituted pyridyl group is exemplified by a dialkylaminocarbonyl group; an alkylaminocarbonyl group wherein the alkyl moiety thereof is optionally substituted by hydroxyl group; an aminocarbonyl group; pyrroridinocarbonyl group; or morpholinocarbonyl group. The substituent(s) of the heterocyclic group of R¹ may be optionally substituted with 1 to 3 substituent(s) on the heterocyclic group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, oxazolidinyl group, tetrahydropyranyl group, tetrahydrothiopyranyl group, dioxanyl group, azetidinyl group, thietanyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. Of these heterocyclic groups, suitably used are piperidinyl group, pyrazinyl group, pyrimidinyl group, pyrrolidinyl group, oxazolidinyl group, tetrahydropyranyl group, tetrahydrothiopyranyl group, dioxanyl group, azetidinyl group or thietanyl group.

In the present invention, R² is hydrogen atom, an optionally substituted hydroxyl group, an optionally substituted amino group, an optionally substituted alkyl group, a substituted carbonyl group or a halogen atom.

In the present invention, the substituent of the optionally substituted hydroxyl group of R² is exemplified by an alkyl group optionally substituted by hydroxyl group.

In the present invention, the substituent of the optionally substituted amino group of R² is exemplified by an alkyl group optionally substituted by hydroxyl group.

In the present invention, the substituent of the optionally substituted alkyl group of R² is an alkoxy group optionally substituted by hydroxyl group or hydroxyl group.

In the present invention, the substituent of the substituted carbonyl group of R² is exemplified by hydroxyl group, an alkoxy group optionally substituted by hydroxyl group or an alkylamino group optionally substituted by hydroxyl group.

In the present invention, Z is exemplified by oxygen atom or a group represented by —N(R³)—.

In the present invention, R³ is exemplified by hydrogen atom or an optionally substituted alkyl group. The substituent of the optionally substituted alkyl group of R³ is exemplified by hydroxyl group, an alkanoyl group, a halogen atom, an alkoxy group or alkylamino group.

In the present invention, R^{4a} and R^{4b} may be the same or different from each other, hydrogen atom, an optionally substituted alkyl group, or may be bonded to each other at the both ends to form an alkylene group. The substituent of the optionally substituted alkyl group is exemplified by hydroxyl group, etc.

As the preferred compound of the present invention, a compound where R¹ is an optionally substituted alkyl group is mentioned. The preferred substituent of the alkyl group is a dialkylaminocarbonyl group wherein the alkyl moiety thereof is optionally substituted by hydroxyl group, morpholinocarbonyl group, hydroxyl group, an alkoxycarbonyl group, an alkanoyl group, an alkylsulfonyl group, alkylimidazolyl group, an alkylpyrazolinyl group, cyano group, carboxyl group, pyrrolidinylsulfonyl group, a halogen atom, an alkylthio group, oxadiazolyl group, a dialkylisoxazolyl group, an oxopyridyl group optionally substituted by an alkyl group or an alkanoylamino group.

As the preferred compound of the present invention, a compound where R¹ is a cycloalkyl group having a substituent(s) is mentioned. The preferred substituent of the cycloalkyl group is hydroxyl group, an alkylenedioxy group or oxo group.

As the preferred compound of the present invention, a compound where R¹ is a substituted carbonyl group is mentioned. The preferred substituent of the carbonyl group is a hydroxyalkyl group; an alkanoylalkyl group; a hydroxycycloalkyl group; an alkylsulfonylalkyl group; an oxopyrrolidinylalkyl group; an oxopyridinylalkyl group substituted by an alkyl group; a morpholinoalkyl group; a thiomorpholinoalkyl group; an aminoalkyl group; tetrahydropyranyloxy group; an alkanoylpiperidinyl group; an alkoxycarbonylpiperidinyl group; an alkylsulfonylpiperidinyl group; pyrimidinylpiperidinyl group; an alkyloxypiperidinyl group; hydroxypiperidinyl group; oxopiperazino group; an alkylpiperazino group; an alkanoylpiperazino group; an alkoxycarbonylpiperazino group; a hydroxyalkylpiperazino group; morpholino group; thiomorpholino group the sulfur atom of which is optionally substituted by 1 or 2 oxo groups; a hydroxyalkylpyrrolidinyl group; an alkyloxopyrrolidinyl group; dioxopyrrolidinyl group; tetrahydropyranyl group; tetrahydrothiopyranyl group the sulfur atom of which is optionally substituted by 2 oxo groups; hydroxyalkylamino group; a dialkylamino group wherein the alkyl moiety thereof is optionally substituted by hydroxyl group; oxopyridyl group; cyanopyridyl group; an alkanoylazetidinyl group; an alkoxycarbonylazetidinyl group; a dialkylaminocarbonylazetidinyl group; an alkylsulfonylazetidinyl group; or an alkoxy group optionally substituted by hydroxyl group.

As the preferred compound of the present invention, a compound where R¹ is a substituted sulfinyl group is mentioned. The substituent of the sulfinyl group is preferably exemplified by an alkyl group optionally substituted by hydroxyl group or hydroxyl group, more preferably an alkyl group optionally substituted by hydroxyl group.

As the preferred compound of the present invention, a compound where R¹ is a substituted sulfonyl group is mentioned. The substituent of the sulfonyl group is preferably exemplified by an alkyl group.

As the preferred compound of the present invention, a compound where R¹ is an optionally substituted heterocyclic group is mentioned. The heterocyclic group is preferably exemplified by piperidinyl group, pyrazinyl group, pyrimidinyl group, pyrrolidinyl group, oxazolidinyl group, tetrahydropyranyl group, tetrahydrothiopyranyl group, dioxanyl group, morpholino group, thiomorpholino group, pyridyl group, azetidinyl group or thietanyl group. Also, the substituent of the heterocyclic group is preferably exemplified by an alkanoyl group optionally substituted by hydroxyl group; an alkoxycarbonyl group; an alkylsulfonyl group; a dialkylaminocarbonyl group; an alkylaminocalbonyl group wherein the alkyl moiety thereof is optionally substituted by hydroxyl group; an aminocarbonyl group; pyrrolidinylcarbonyl group; morpholinocarbonyl group; a cycloalkyl group substituted by hydroxyl group; an alkyl group; a trihalogenoalkyl group; hydroxyl group; or oxo group; etc. The heterocyclic group may have 1 to 3 substituent(s).

As the compound [I] of the present invention, a compound where Ring A is a benzene ring represented by the formula:

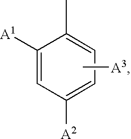

Ring B is a benzene ring represented by the formula:

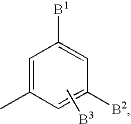

A¹ is hydrogen atom, a halogen atom, an alkyl group or an alkoxy group, A² is hydrogen atom or a halogen atom, A³ is hydrogen atom, B¹ is hydrogen atom, an alkyl group, a halogen atom, cyano group, an alkoxy group or a trihalogenoalkyl group, B² is hydrogen atom, an alkyl group, a halogen atom, cyano group, an alkoxy group or a trihalogenoalkyl group, B³ is hydrogen atom, R¹ is hydrogen atom; an alkyl group optionally substituted by a dialkylaminocarbonyl group wherein the alkyl moiety thereof is optionally substituted by hydroxyl group, morpholinocarbonyl group, hydroxyl group, an alkoxycarbonyl group, morpholinoaminocarbonyl group, a hydroxyalkylaminocarbonyloxy group, an alkylpiperazinocarbonyl group, an alkanoyl group, an alkylsulfonyl group, an alkylimidazolyl group, an alkylpyrazolinyl group, cyano group, carboxyl group, pyrrolidinylsulfonyl group, a halogen atom, an alkylthio group, oxadiazolyl group, a dialkylisoxazolyl group, oxopyridyl group optionally substituted by an alkyl group, an alkanoylamino group or a hydroxyalkylaminocarbonyl group; a trihalogenoalkyl group; a cycloalkyl group optionally substituted by hydroxyl group, an alkylenedioxy group or oxo group; carboxyl group; an alkanoyl group optionally substituted by hydroxyl group, an alkanoyl group, an alkylsulfonyl group, oxopyrrolidinyl group, pyrrolidinyl group substituted by an alkyl group and oxo group, morpholino group, thiomorpholino group or amino group; an alkoxycarbonyl group optionally substituted by hydroxyl group; tetrahydropyranyloxycarbonyl group; pyrimidinylaminocarbonyl group; an alkylaminocarbonyl group wherein the alkyl moiety thereof is optionally substituted by hydroxyl group or cyano group; a dialkylaminocarbonyl group wherein the alkyl moiety thereof is optionally substituted by 1 or 2 hydroxyl group(s); pyridylaminocarbonyl group wherein the pyridyl moiety thereof is substituted by hydroxyl group; aminocarbonyl group substituted by an alkylpyridonyl group and an alkyl group; piperidinylcarbonyl group substituted by 1 or 2 substituent(s) selected from an alkanoyl group, hydroxyl group, oxo group, an alkoxycarbonyl group, an alkylsulfonyl group, pyrimidinyl group and an alkyl group; piperazinocarbonyl group substituted by oxo group, an alkyl group, pyrimidinyl group, an alkylsulfonyl group, an alkanoyl group, an alkoxycarbonyl group or hydroxyalkyl group; morpholinocarbonyl group; thiomorpholinocarbonyl group the sulfur atom of which is optionally substituted by 1 or 2 oxo group(s); pyrrolidinylcarbonyl group substituted by a hydroxyalkyl group or hydroxyl group; a cycloalkylcarbonyl group substituted by 1 or 2 substituent(s) selected from hydroxyl group, an alkyl group, oxo group, an alkoxycarbonyl group or oxopyrrolidinyl group; oxopyrrolidinylcarbonyl group optionally substituted by an alkyl group or oxo group; tetrahydropyranylcarbonyl group; tetrahydrothiopyranylcarbonyl group the sulfur atom of which is optionally di-substituted by oxo groups; pyridylcarbonyl group substituted by oxo group or cyano group; an azetidinylcarbonyl group substituted by an alkanoyl group, an alkoxycarbonyl group, a dialkylaminocarbonyl group, an alkylsulfonyl group or a trihalogenoalkyl group; an alkylsulfinyl group optionally substituted by hydroxyl group; hydroxysulfinyl group; an alkylsulfonyl group optionally substituted by hydroxyl group or an alkanoyloxy group; piperidinyl group substituted by an alkanoyl group, an alkoxycarbonyl group or an alkylsulfonyl group; tetrahydropyranyl group; tetrahydrothiopyranyl group the sulfur atom of which is optionally substituted by 1 or 2 oxo groups; dialkyldioxanyl group; dioxothiomorpholino group; morpholino group optionally disubstituted by oxo group; oxopyrrolidinyl group; dioxopyrrolidinyl group optionally substituted by an alkyl group; azetidinyl group substituted by an alkanoyl group optionally substituted by hydroxyl group, an alkoxycarbonyl group, an alkylsulfonyl group, a dialkylaminocarbonyl group, a trihalogenoalkyl group or a cycloalkylcarbonyl group substituted by hydroxyl group; thietanyl group the sulfur atom of which is substituted by 1 or 2 oxo group(s); pyrazinyl group; pyrimidinyl group; oxo-oxazolidinyl group; or a pyridyl group substituted by a dialkylaminocarbonyl group, an alkylaminocarbonyl group wherein the alkyl moiety thereof is optionally substituted by hydroxyl group, an aminocarbonyl group, pyrrolidinylcarbonyl group or morpholinocarbonyl group, R² is hydrogen atom, Z is oxygen atom or a group represented by —N(R³)—, R³ is an alkyl group optionally substituted by hydroxyl group, R^{4a} is hydrogen atom or an alkyl group optionally substituted by hydroxyl group, R^{4b} is hydrogen atom or an alkyl group optionally substituted by hydroxyl group.

Of these, preferred are compounds wherein Ring A is a benzene ring represented by the formula:

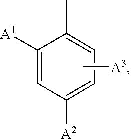

Ring B is a benzene ring represented by the formula:

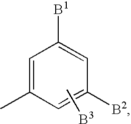

A¹ is hydrogen atom, a halogen atom or alkyl group, A² is hydrogen atom or a halogen atom, A³ is hydrogen atom, B¹ is a trihalogenoalkyl group, an alkyl group, an alkoxy group or a halogen atom, B 2 is hydrogen atom, a trihalogenalkyl group, an alkyl group, an alkoxy group or a halogen atom, B³ is hydrogen atom or a halogen atom, R¹ is hydrogen atom; an alkyl group optionally substituted by a dialkylaminocarbonyl group wherein the alkyl moiety thereof is optionally substituted by hydroxyl group, morpholinocarbonyl group, hydroxyl group, an alkoxycarbonyl group, an alkanoyl group, an alkylsulfonyl group, an alkylimidazolyl group, an alkylpyrazolinyl group, cyano group, carboxyl group, pyrrolidinylsulfonyl group, a halogen atom, an alkylthio group, oxadiazolyl group, a dialkylisoxazolyl group, oxopyridyl group optionally substituted by an alkyl group or an alkanoylamino group; a trihalogenoalkyl group; a cycloalkyl group optionally substituted by hydroxyl group, an alkylenedioxy group or oxo group; an alkanoyl group substituted by hydroxyl group, an alkanoyl group, an alkylsulfonyl group, oxopyrrolidinyl group, pyrrolidinyl group substituted by an alkyl group and oxo group, morpholino group, thiomorpholino group or amino group; an alkoxycarbonyl group optionally substituted by hydroxyl group; tetrahydropyranyloxycarbonyl group; an alkylaminocarbonyl group wherein the alkyl moiety thereof is optionally substituted by hydroxyl group; a dialkylaminocarbonyl group wherein the alkyl moiety thereof is optionally substituted by 1 or 2 hydroxyl group(s); piperidinylcarbonyl group substituted by 1 or 2 substituent(s) selected from an alkanoyl group, hydroxyl group, oxo group, an alkoxycarbonyl group, an alkylsulfonyl group, pyrimidinyl group and an alkyl group; piperazinocarbonyl group substituted by oxo group, an alkyl group, an alkanoyl group, an alkoxycarbonyl group or hydroxyalkyl group; morpholinocarbonyl group; thiomorpholinocarbonyl group the sulfur atom of which is optionally substituted by 1 or 2 oxo group(s); pyrrolidinylcarbonyl group substituted by a hydroxyalkyl group or hydroxyl group; a cycloalkylcarbonyl group substituted by 1 or 2 substituent(s) selected from hydroxyl group, an alkyl group, oxo group, an alkoxycarbonyl group and oxopyrrolidinyl group; oxopyrrolidinylcarbonyl group optionally substituted by an alkyl group or oxo group; tetrahydropyranylcarbonyl group; tetrahydrothiopyranylcarbonyl group the sulfur atom of which is optionally di-substituted by oxo groups; pyridylcarbonyl group substituted by oxo group or cyano group; azetidinylcarbonyl group substituted by an alkanoyl group, an alkoxycarbonyl group, a dialkylaminocarbonyl group or an alkylsulfonyl group; an alkylsulfinyl group; an alkylsulfonyl group; piperidinyl group substituted by an alkanoyl group, an alkoxycarbonyl group or an alkylsulfonyl group; tetrahydropyranyl group; tetrahydrothiopyranyl group the sulfur atom of which is optionally di-substituted by oxo groups; dialkyldioxanyl group; dioxothiomorpholino group; morpholino group optionally disubstituted by oxo group; oxopyrrolidinyl group; dioxopyrrolidinyl group optionally substituted by an alkyl group; azetidinyl group substituted by an alkanoyl group optionally substituted by hydroxyl group, an alkoxycarbonyl group, an alkylsulfonyl group, a dialkylaminocarbonyl group a trihalogenoalkyl group or a cycloalkylcarbonyl group substituted by hydroxyl group; thietanyl group the sulfur atom of which is substituted by 1 or 2 oxo group(s); pyrazinyl group; pyrimidinyl group; oxoxazolidinyl group; or a pyridyl group substituted by a dialkylaminocarbonyl group, an alkylaminocarbonyl group wherein the alkyl moiety thereof is optionally substituted by hydroxyl group, an aminocarbonyl group, pyrrolidinylcarbonyl group or morpholinocarbonyl group, R² is hydrogen atom, Z is a group represented by —N(R³)—, R³ is an alkyl group, R^{4a} is hydrogen atom or an alkyl group, R^{4b} is hydrogen atom or an alkyl group.

Moreover, preferred are compounds wherein Ring A is a benzene ring represented by the formula:

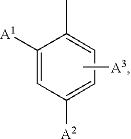

Ring B is a benzene ring represented by the formula:

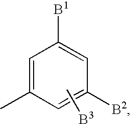

A¹ is hydrogen atom or an alkyl group, A² is a halogen atom, A³ is hydrogen atom, B¹ is a trihalogenomethyl group, B² is a trihalogenomethyl group, B³ is hydrogen atom, R¹ is an alkyl group substituted by oxopyridyl group optionally substituted by an alkyl group, a dialkylaminocarbonyl group or an alkoxycarbonyl group; an alkanoyl group substituted by hydroxyl group; an alkoxycarbonyl group substituted by hydroxyl group; an alkylaminocarbonyl group wherein the alkyl moiety thereof is optionally substituted by hydroxyl group; piperidinylcarbonyl group substituted by an alkanoyl group, an alkoxycarbonyl group or an alkylsulfonyl group; piperazinecarbonyl group substituted by an alkanoyl group; a cycloalkylcarbonyl group substituted by hydroxyl group and an alkyl group; tetrahydropyranylcarbonyl group; azetidinylcarbonyl group substituted by an alkoxycarbonyl group or an alkylsulfonyl group; piperidinyl group substituted by an alkanoyl group or an alkoxycarbonyl group; tetrahydropyranyl group; tetrahydrothiopyranyl group the sulfur atom of which is optionally di-substituted by oxo groups; dioxothiomorpholino group; oxopyrrolidinyl group; dioxopyrrolidinyl group; azetidinyl group substituted by an alkanoyl group, an alkoxycarbonyl group, an alkylsulfonyl group or dialkylaminocarbonyl group; thietanyl group the sulfur atom of which is substituted by 1 or 2 oxo group(s); or oxo-oxazolidinyl group, R² is hydrogen atom, Z is a group represented by the formula —N(R³)—, R³ is an alkyl group, R^{4a} is hydrogen atom or an alkyl group, R^{4b} is hydrogen atom or an alkyl group.

Furthermore, in the compounds of the present invention, preferred compounds are a compound selected from the following (A) to (BD) or a pharmaceutically acceptable salt thereof.
(A) (3S,4S)-1-(acetylpiperidin-4-yl)carbonyl-4-{N-1-(R)-(3,5-bistrifluoromethylphenyl)ethyl-N-methyl}aminocarbonyl-3-(4-fluoro-2-methylphenyl)piperidine,
(B) (3S,4S)-1-(1-acetylpiperidin-4-yl)-4-{N-1-(R)-(3,5-bistrifluoromethylphenyl)ethyl-N-methyl}aminocarbonyl-3-(4-fluoro-2-methylphenyl)piperidine,
(C) (3S,4S)-1-(1-acetylpiperidin-4-yl)-4-{N-1-(S)-(3,5-bistrifluoromethylphenyl)ethyl-N-methyl}aminocarbonyl-3-(4-fluoro-2-methylphenyl)piperidine,
(D) (3S,4S)-4-{N-1-(S)-(3,5-bistrifluoromethylphenyl)ethyl-Nmethyl}aminocarbonyl-3-(4-fluoro-2-methylphenyl)-1-(3-hydroxy-3-methylbutyryl)piperidine,
(E) (3S,4S)-4-{N-1-(S)-(3,5-bistrifluoromethylphenyl)ethyl-Nmethyl}aminocarbonyl-3-(4-fluoro-2-methylphenyl)-1-{3-(S)-hydroxybutyryl}piperidine,
(F) (3S,4S)-4-{N-1-(R)-(3,5-bistrifluoromethylphenyl)ethyl-Nmethyl}aminocarbonyl-3-(4-fluoro-2-methylphenyl)-1-{3-(S)-hydroxybutyryl}piperidine,
(G) (3S,4S)-4-{N-1-(R)-(3,5-bistrifluoromethylphenyl)ethyl-Nmethyl}aminocarbonyl-1-(1,1-dioxotetrahydrothiopyran-4-yl)-2-(4-fluoro-2-methylphenyl)piperidine,
(H) (3S,4S)-4-{N-1-(S)-(3,5-bistrifluoromethylphenyl)ethyl-Nmethyl}aminocarbonyl-1-(1,1-dioxotetrahydrothiopyran-4-yl)-2-(4-fluoro-2-methylphenyl)piperidine,
(I) (3S,4S)-1-(1-propionylpiperidin-3-yl)carbonyl-4-{N-1-(R)-(3,5-bistrifluoromethylphenyl)ethyl-N-methyl}aminocarbonyl-3-(4-fluoro-2-methylphenyl)piperidine,
(J) (3S,4S)-4-{N-1-(R)-(3,5-bistrifluoromethylphenyl)ethyl-Nmethyl}aminocarbonyl-3-(4-fluoro-2-methylphenyl)-1-(1-methoxycarbonylpiperidin-3-yl)piperidine,
(K) (3S,4S)-4-{N-1-(S)-(3,5-bistrifluoromethylphenyl)ethyl-N-methyl}aminocarbonyl-3-(4-fluoro-2-methylphenyl)-1-(1-methoxycarbonylpiperidin-3-yl)piperidine,
(L) (3S,4S)-4-{N-1-(R)-(3,5-bistrifluoromethylphenyl)ethyl-N-methyl}aminocarbonyl-3-(4-fluoro-2-methylphenyl)-1-hydroxyacetylpiperidine,
(M) (3S,4S)-4-{N-1-(S)-(3,5-bistrifluoromethylphenyl)ethyl-N-methyl}aminocarbonyl-3-(4-fluoro-2-methylphenyl)-1-(2-hydroxy-2-methylpropionyl)piperidine,
(N) (3S,4S)-4-{N-1-(S)-(3,5-bistrifluoromethylphenyl)ethyl-N-methyl}aminocarbonyl-3-(4-fluoro-2-methylphenyl)-1-{2-(R)-hydroxypropylaminocarbonyl}piperidine,
(O) (3S,4S)-4-{N-1-(S)-(3,5-bistrifluoromethylphenyl)ethyl-N-methyl}aminocarbonyl-3-(4-fluoro-2-methylphenyl)-1-{2-(S)-hydroxypropylaminocarbonyl}piperidine,
(P) (3S,4S)-1-(4-acetylpiperazinocarbonyl)-4-{N-1-(R)-(3,5-bistrifluoromethylphenyl)ethyl-N-methyl}aminocarbonyl-3-(4-fluoro-2-methylphenyl)piperidine,
(Q) (3S,4S)-4-{N-1-(R)-(3,5-bistrifluoromethylphenyl)ethyl-N-methyl}aminocarbonyl-3-(4-fluoro-2-methylphenyl)-1-(1-methoxycarbonylpiperidin-4-yl)piperidine,
(R) (3S,4S)-4-{N-1-(S)-(3,5-bistrifluoromethylphenyl)ethyl-N-methyl}aminocarbonyl-3-(4-fluoro-2-methylphenyl)-1-(1-methoxycarbonylpiperidin-4-yl)piperidine,
(S) (3S,4S)-1-(1-acetylazetidin-3-yl)-4-{N-1-(R)-(3,5-bistrifluoromethylphenyl)ethyl-N-methyl}aminocarbonyl-3-(4-fluoro-2-methylphenyl)piperidine,
(T) (3S,4S)-1-(1-acetylazetidin-3-yl)-4-{N-1-(S)-(3,5-bistrifluoromethylphenyl)ethyl-N-methyl}aminocarbonyl-3-(4-fluoro-2-methylphenyl)piperidine,
(U) (3S,4S)-4-{N-1-(R)-(3,5-bistrifluoromethylphenyl)ethyl-Nmethyl}aminocarbonyl-3-(4-fluoro-2-methylphenyl)-1-(1-propionyl-azetidin-3-yl)piperidine,
(V) (3S,4S)-4-{N-1-(S)-(3,5-bistrifluoromethylphenyl)ethyl-Nmethyl}aminocarbonyl-3-(4-fluoro-2-methylphenyl)-1-(1-propionyl-azetidin-3-yl)piperidine,
(W) (3S,4S)-4-{N-1-(R)-(3,5-bistrifluoromethylphenyl)ethyl-Nmethyl}aminocarbonyl-3-(4-fluoro-2-methylphenyl)-1-(1-methoxycarbonylazetidin-3-yl)piperidine,
(X) (3S,4S)-4-{N-1-(S)-(3,5-bistrifluoromethylphenyl)ethyl-N-methyl}aminocarbonyl-3-(4-fluoro-2-methylphenyl)-1-(1-methoxycarbonylazetidin-3-yl)piperidine,
(Y) (3S,4S)-4-{N-1-(R)-(3,5-bistrifluoromethylphenyl)ethyl-Nmethyl}aminocarbonyl-3-(4-fluoro-2-methylphenyl)-1-(1-methanesulfonylazetidin-3-yl)piperidine,
(Z) (3S,4S)-4-{N-1-(S)-(3,5-bistrifluoromethylphenyl)ethyl-N-methyl}aminocarbonyl-3-(4-fluoro-2-methylphenyl)-1-(1-methanesulfonylazetidin-3-yl)piperidine,
(AA) (3S,4S)-4-{N-1-(R)-(3,5-bistrifluoromethylphenyl)ethyl-Nmethyl}aminocarbonyl-3-(4-fluoro-2-methylphenyl)-1-(1-dimethylaminocarbonylazetidin-3-yl)piperidine,
(AB) (3S,4S)-4-{N-1-(S)-(3,5-bistrifluoromethylphenyl)ethyl-Nmethyl}aminocarbonyl-3-(4-fluoro-2-methylphenyl)-1-(1-dimethylaminocarbonylazetidin-3-yl)piperidine,
(AC) (3S,4S)-4-{N—(S)-2-(3,5-bistrifluoromethylphenyl)ethyl-Nmethyl}aminocarbonyl-3-(4-fluoro-2-methylphenyl)-1-(1-oxothiethan-3-yl)piperidine,
(AD) (3S,4S)-4-{N—(R)-2-(3,5-bistrifluoromethylphenyl)ethyl-Nmethyl}aminocarbonyl-3-(4-fluoro-2-methylphenyl)-1-(1,1-dioxothiethan-3-yl)piperidine,
(AE) (3S,4S)-4-{N—(S)-2-(3,5-bistrifluoromethylphenyl)ethyl-Nmethyl}aminocarbonyl-3-(4-fluoro-2-methylphenyl)-1-(1,1-dioxothiethan-3-yl)piperidine,
(AF) (3S,4S)-4-{N-1-(S)-(3,5-bistrifluoromethylphenyl)ethyl-Nmethyl}aminocarbonyl-2-(4-fluoro-2-methylphenyl)-1-(tetrahydrothiopyran-4-yl)piperidine,
(AG) (3S,4S)-4-{N-1-(R)-(3,5-bistrifluoromethylphenyl)ethyl-Nmethyl}aminocarbonyl-2-(4-fluoro-2-methylphenyl)-1-(tetrahydropyran-4-yl)piperidine,
(AH) (3S,4S)-4-{N-1-(S)-(3,5-bistrifluoromethylphenyl)ethyl-Nmethyl}aminocarbonyl-2-(4-fluoro-2-methylphenyl)-1-(tetrahydropyran-4-yl)piperidine,
(AI) (3S,4S)-4-{N-1-(R)-(3,5-bistrifluoromethylphenyl)ethyl-Nmethyl}aminocarbonyl-2-(4-fluoro-2-methylphenyl)-1-(1-oxypyridin-4-yl)methylpiperidine,
(AJ) (3S,4S)-4-{N-1-(S)-(3,5-bistrifluoromethylphenyl)ethyl-Nmethyl}aminocarbonyl-2-(4-fluoro-2-methylphenyl)-1-(1-oxypyridin-4-yl)methylpiperidine,
(AK) (3S,4S)-4-{N-1-(R)-(3,5-bistrifluoromethylphenyl)ethyl-Nmethyl}aminocarbonyl-2-(4-fluoro-2-methylphenyl)-1-(2-methyl-1-oxypyridin-5-yl)methylpiperidine,
(AL) (3S,4S)-4-{N-1-(S)-(3,5-bistrifluoromethylphenyl)ethyl-Nmethyl}aminocarbonyl-2-(4-fluoro-2-methylphenyl)-1-(2-oxopyrrolidin-1-yl)piperidine,
(AM) (3S,4S)-4-{N-1-(S)-(3,5-bistrifluoromethylphenyl)ethyl-N-methyl}aminocarbonyl-2-(4-fluoro-2-methylphenyl)-1-(2-oxooxazolidin-3-yl)piperidine,
(AN) (3S,4S)-4-{N-1-(R)-(3,5-bistrifluoromethylphenyl)ethyl-Nmethyl}aminocarbonyl-1-(2,4-dioxopyrrolidin-1-yl)-2-(4-fluoro-2-methylphenyl)piperidine,
(AO) (3S,4S)-4-{N-1-(S)-(3,5-bistrifluoromethylphenyl)ethyl-N-methyl}aminocarbonyl-1-(2,4-dioxopyrrolidin-1-yl)-2-(4-fluoro-2-methylphenyl)piperidine,
(AP) (3S,4S)-4-{N-1-(R)-(3,5-bistrifluoromethylphenyl)ethyl-Nmethyl}aminocarbonyl-1-(1,1-dioxothiomorpholin-4-yl)-2-(4-fluoro-2-methylphenyl)piperidine,
(AQ) (3S,4S)-4-{N-1-(S)-(3,5-bistrifluoromethylphenyl)ethyl-Nmethyl}aminocarbonyl-1-(1,1-dioxothiomorpholin-4-yl)-2-(4-fluoro-2-methylphenyl)piperidine,
(AR) (3S,4S)-4-{N-1-(R)-(3,5-bistrifluoromethylphenyl)ethyl-Nmethyl}aminocarbonyl-2-(4-fluoro-2-methylphenyl)-1-(trans-4-hydroxy-4-methylcyclohexylcarbonyl)piperidine,
(AS) (3S,4S)-4-{N-1-(S)-(3,5-bistrifluoromethylphenyl)ethyl-N-methyl}aminocarbonyl-2-(4-fluoro-2-methylphenyl)-1-(trans-4-hydroxy-4-methylcyclohexylcarbonyl)piperidine,
(AT) (3S,4S)-4-{N-1-(R)-(3,5-bistrifluoromethylphenyl)ethyl-N-methyl}aminocarbonyl-2-(4-fluoro-2-methylphenyl)-1-(1-methoxycarbonylazetidin-3-ylcarbonyl)piperidine,
(AU) (3S,4S)-4-{N-1-(S)-(3,5-bistrifluoromethylphenyl)ethyl-Nmethyl}aminocarbonyl-2-(4-fluoro-2-methylphenyl)-1-(1-methoxycarbonylazetidin-3-ylcarbonyl)piperidine,
(AV) (3S,4S)-4-{N-1-(S)-(3,5-bistrifluoromethylphenyl)ethyl-N-methyl}aminocarbonyl-1-ethylaminocarbonyl-2-(4-fluoro-2-methylphenyl)piperidine,
(AW) (3S,4S)-4-{N-1-(S)-(3,5-bistrifluoromethylphenyl)ethyl-Nmethyl}aminocarbonyl-2-(4-fluoro-2-methylphenyl)-1-(1-methylethylaminocarbonyl)piperidine,
(AX) (3S,4S)-4-{N—(S)-2-(3,5-bistrifluoromethylphenyl)ethyl-Nmethyl}aminocarbonyl-3-(4-fluoro-2-methylphenyl)-1-(2-hydroxyethyloxycarbonyl)piperidine,
(AY) (3S,4S)-4-{(N—(S)-2-(3,5-bistrifluoromethylphenyl)ethyl-Nmethyl}aminocarbonyl-1-dimethylaminocarbonylmethyl-3-(4-fluoro-2-methylphenyl)piperidine,
(AZ) (3S,4S)-4-{N—(S)-2-(3,5-bistrifluoromethylphenyl)ethyl-N-methyl}aminocarbonyl-1-dimethylaminocarbonylethyl-3-(4-fluoro-2-methylphenyl)piperidine,
(BA) (3S,4S)-4-{N-1-(R)-(3,5-bistrifluoromethylphenyl)ethyl-N-methyl}aminocarbonyl-3-(4-fluoro-2-methylphenyl)-1-(1-methanesulfonylpiperidine-4-yl)piperidine,
(BB) (3S,4S)-4-{N-1-(R)-(3,5-bistrifluoromethylphenyl)ethyl-Nmethyl}aminocarbonyl-3-(4-fluoro-2-methylphenyl)-1-{1-(2-methylpropionyl)piperidin-4-ylcarbonyl}piperidine,
(BC) (3S,4S)-4-{N-1-(S)-(3,5-bistrifluoromethylphenyl)ethyl-N-methyl}aminocarbonyl-3-(4-fluoro-2-methylphenyl)-1-(tetrahydropyran-4-ylcarbonyl)piperidine, and
(BD) (3S,4S)-4-{N—(S)-(3,5-bistrifluoromethylphenyl)ethyl-N-methyl}aminocarbonyl-3-(4-fluoro-2-methylphenyl)-1-{(R)-1-methoxycarbonyl}ethyl]piperidine.

Another preferred compounds are a compound selected from the following (a) to (c) or a pharmaceutically acceptable salt thereof.
(a) (3S,4S)-4-{N-1-(R)-(3,5-bistrifluoromethylphenyl)ethyl-N-methyl}aminocarbonyl-3-(4-fluoro-2-methylphenyl)-1-{1-(3-hydroxy-3-methylbutyryl)azetidin-3-yl}piperidine,
(b) (3S,4S)-4-{N-1-(S)-(3,5-bistrifluoromethylphenyl)ethyl-Nmethyl}aminocarbonyl-3-(4-fluoro-2-methylphenyl)-1-{1-(3-hydroxy-3-methylbutyryl)azetidin-3-yl}piperidine, and
(c) (3S,4S)-4-{N-1-(R)-(3,5-bistrifluoromethylphenyl)ethyl-Nmethyl}aminocarbonyl-3-(4-fluoro-2-methylphenyl)-1-{1-(3,3,3-trifuluoropropyl)azetidin-3-yl}piperidine.

The compound [I] of the present invention can be used for a pharmaceutical use either in a free form or in form of a pharmaceutically acceptable salt.

As the pharmaceutically acceptable salt of the compound [I] of the present invention, there may be mentioned, for example, an inorganic acid salt such as hydrochloride, sulfate, phosphate and hydrobromide; and an organic acid salt such as acetate, fumarate, oxalate, citrate, methanesulfonate, benzenesulfonate, tosylate, maleate, succinate and tartarate.

Further, the compound [I] of the present invention or a pharmaceutically acceptable salt thereof includes any of its internal salts, solvates and hydrates, etc.

Although an optical isomer based on an asymmetric carbon can be present in the compound [I] of the present invention, the present invention includes any of these optical isomers and the mixture thereof. In the present invention, among these optical isomers, preferred is a compound having S configuration at 3-position of the piperidine ring (the connecting position of Ring A), and particularly preferred is a compound having S configuration at 3-position of the piperidine ring (the connecting position of Ring A) and S configuration at 4-position of the piperidine ring.

The compound [I] or a pharmaceutically acceptable salt thereof of the present invention has an excellent tachykinin receptor antagonistic action, particularly an SP receptor antagonistic action, whereby it is useful as a safe medicament for prophylaxis and treatment for inflammation or allergic diseases (for example, atopic dermatitis, dermatitis, herpes, proriasis, asthma, bronchitis, expectoration, rhinitis, rheumatoid arthritis, osteoarthritis, osteoporosis, multiple sclerosis, conjunctivitis, ophthalmia, cystitis, etc.), pain, migraine, neuralgia, itchiness, cough, and further central nervous system diseases (for example, schizophrenia, Parkinson's disease, depression, uneasiness, psychosomatic disorder, morphine dependence, dementia (for example, Alzheimer's disease, etc.), etc.), digestive organs disease (for example, irritable bowel syndrome, ulcerative colitis, Crohn's disease, disorder (for example, gastritis, gastric ulcer, etc.) related to urease-positive Spirillum (for example, helicobacter pylori, etc.), etc.), nausea, emesis, urinary disorder (for example, pollakiurea, urinary incontinence, etc.), circulatory disease (for example, angina pectoris, hypertension, cardiac failure, thrombosis, etc.) and immune disorder, etc. in mammals (for example, mouse, guinea pig, Mongolian gerbil, ferret, rat, hamster, rabbit, cat, dog, bovine, sheep, monkey, human, etc.). Particularly, since compound [I] or a pharmaceutically acceptable salt thereof which is an active ingredient of the present invention has a high penetration to the brain and has a low toxicity (high safety), showing almost no side effect, it is useful as a therapeutic or prophylactic agent for central nervous system diseases such as emesis, depression and so forth, or urinary disorder such as pollakiuria, etc.

Measurements on the compound of the present invention or a pharmaceutically acceptable salt thereof can be carried out, according to the method described in European Journal of Pharmacology, vol. 254, pages 221-227 (1994) with respect to a neurokinin-1 receptor binding action, and according to the method described in European Journal of Pharmacology, vol. 265, pages 179-183 (1994) with respect to neurokinin-1 receptor antagonstic action, according to the method described in Journal of Urology, vol. 155, No. 1, pages 355-360 (1996) with regard to an inhibitory action on pollakiuria.

The compound [I] or a pharmaceutically acceptable salt thereof of the present invention can be administered orally or parenterally, and it can be formulated into a suitable preparation, using a conventionally used pharmaceutical carrier for an oral or parenteral administration. As such a pharmaceutical carrier, there may be mentioned, for example, a binder (syrup, Gum Arabic, gelatin, sorbitol, tragacanth, polyvinylpyrrolidone, etc.), an excipient (lactose, sugar, corn starch, potassium phosphate, sorbitol, glycine, etc.), a lubricant (magnesium stearate, talc, polyethylene glycol, silica, etc.), a disintegrator (potato starch, etc.) and a wetting agent (anhydrous lauryl sodium sulfate, etc.), and the like.

Also, when these pharmaceutical preparations are administered orally, they may be a solid preparation such as tablets, granules, capsules and powders, or a liquid preparation such as solution, suspension and emulsion. On the other hand, when they are administered parenterally, for example, they can be administered as an injection solution or an infusion solution, using distilled water for injection, physiological saline, aqueous glucose solution, etc., or they may be administered as a suppository, and the like.

A dose of the compound [I] or a pharmaceutically acceptable salt thereof of the present invention may vary depending on an administration method, an age, a body weight or a condition of a patient, etc., and, for example, in case of oral administration, it is usually administered in a dose of 0.1 to 20 mg/kg per day, and particularly preferably 0.1 to 10 mg/kg per day, and in case of parenteral administration, usually in a dose of 0.01 to 10 mg/kg per day, particularly preferably 0.01 to 1 mg/kg per day.

### [Method A]

The compound of the formula [I]:

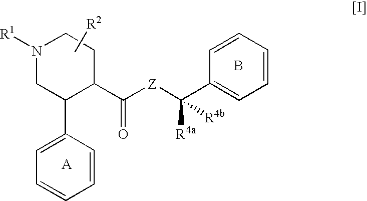

wherein Ring A represents an optionally substituted benzene ring,
Ring B represents an optionally substituted benzene ring,
R¹ represents hydrogen atom or a substituent for the amino group,
R² represents hydrogen atom, an optionally substituted hydroxyl group, an optionally substituted amino group, an optionally substituted alkyl group, a substituted carbonyl group or a halogen atom,
Z represents oxygen atom or a group represented by —N(R³)—,
R³ represents hydrogen atom or an optionally substituted alkyl group,
R^{4a} and R^{4b} may be the same or different from each other, and each is hydrogen atom or an optionally substituted alkyl group, or may be bonded to each other at the both ends to form an alkylene group,
   according to the present invention can be prepared, for example, by reacting the compound of the formula [II]:

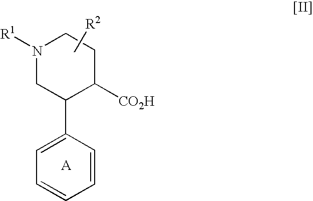

wherein Ring A, R¹ and R² have the same meanings as defined above,
   with the compound of the formula [III]:

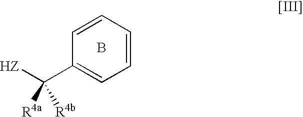

wherein Ring B, Z, R^{4a} and R^{4b} have the same meanings as defined above.

This [Method A] can be carried out as mentioned below.

### [Method A]

The reaction of Compound [II] with Compound [III] can be carried out in a solvent in the presence of a condensing agent; reacting a reactive derivative (acyl halide, acid anhydride, active amide, active ester, mixed acid anhydride, etc.) of Compound [II] with Compound [III] in a solvent in the presence or absence of a base; or reacting an active ester of Compound [II] with Compound [III] in a solvent in the presence of a condensing agent, to prepare a target compound. As the base, organic bases such as pyridine, 4-dimethylaminopyridine, N-methylmorpholine, triethylamine, N,N-dimethylaniline, N,N-diethylaniline, 1,8-diazabicyclo-[5.4.0]undec-7-ene, etc., inorganic bases such as sodium hydrogen carbonate, potassium hydrogen carbonate, sodium carbonate, potassium carbonate, etc. can be used. As the condensing agent, 1,1′-carbonyldiimidazole, 1,3-dicyclohexylcarbodiimide, 1-ethyl-3-(3-dimethylaminopropyl)carbodiimide hydrochloride, propanephosphonic acid anhydride, etc. can be used. Any solvent can be used as long as it does not exert any bad effect on the reaction, for example, N,N-dimethylformamide, dichloromethane, tetrahydrofuran, dioxane, ethyl acetate, 1,3-dimethyl-2-imidazolidinone, etc. can be used. This reaction suitably proceeds, for example, at −20° C. to 60° C., particularly preferably at 5° C. to 60° C. As the active ester of Compound [II], an ester with N-hydroxysuccinic imide, N-hydroxyphthalimide, 1-hydroxybenzotriazole or p-nitrophenol can be used. As the acyl halide of Compound [II], an acyl chloride, an acyl bromide, etc., can be suitably used. Also, as the active amide of Compound [II], an amide with imidazole, etc. can be used.

The objective Compound [I] of the present invention can be also prepared by converting the group R¹ of the compound obtained as mentioned above into the other substituent. Such a converting method of the substituent can be suitably selected depending on the kinds of the substituents to be converted, for example, it can be carried out by the following (Method a) to (Method p).

(Method a): The objective Compound [I] in which the group R¹ in the formula [I] is hydrogen atom can be prepared by eliminating a protective group from a corresponding Compound [I] in which the group R¹ is the protective group for the amino group. Removal of the protective group can be carried out by the conventional manner (for example, acid treatment, base treatment, catalytic reduction, etc.). Among the present reactions, a reaction by the acid treatment can be carried out, for example, at 5° C. to 120° C., a reaction by the base treatment at 5° C. to 40° C., and a reaction by the catalytic reduction at 10° C. to 40° C.
(Method b): The objective Compound [I] in which the group R¹ in the formula [I] is a substituted carbonyl group can be prepared by reacting a corresponding Compound [I] in which the group R¹ is hydrogen atom with the corresponding carboxylic acid compound or its active ester, or carboxylic halide in the presence or in the absence of a condensing agent. As the condensing agent, 1,1′-carbonyldiimidazole, 1,3-dicyclohexylcarbodiimide, 1-ethyl-3-(3-dimethylaminopropyl)carbodiimide hydrochloride, isobutyl chloroformate or N-methylmorpholine, etc., can be used, which are compounds normally used in a reaction to form an amide bond from a carboxylic acid and an amine. As the active ester of the carboxylic acid compound, an ester with N-hydroxysuccinic imide, N-hydroxyphthalimide, 1-hydroxybenzotriazole or p-nitrophenol can be used. This reaction can be carried out, for example, at −20° C. to 50° C.
(Method c): The objective Compound [I] in which the group R¹ in the formula [I] is an optionally substituted heterocyclic group can be prepared by subjecting a corresponding Compound [I] in which the group R¹ is hydrogen atom and a heterocyclic group having a corresponding oxo group to reductive condensation. The reductive condensation can be suitably carried out, for example, according to the method disclosed in (a) Tetrahedron Letters, vol. 31, p. 5595, 1990, (b) Journal of Organic Chemistry, vol. 28, p. 3259, 1963, etc., in the presence of a reducing agent in a suitable solvent. As the reducing agent, any materials which can be suitably used in the reductive amination can be used. Such a reducing agent can be exemplified by a metal reducing agent, for example, metal hydrides [borane hydrides (diborane, etc.)], metal hydride complexes [lithium aluminum hydride, sodium borohydride, etc.], organometal complexes [borane-methyl sulfide, 9-borabicyclononane (9-BBN), triethylsilane, sodium triacetoxyborohydride, sodium cyanoborohydride, etc.] and the like. Also, if necessary, a Lewis acid (titanium tetrachloride, etc.) can be used as an additive. Also, in the reductive condensation, it can be also carried out under catalytic hydrogenation conditions in place of existing the reducing agent. For example, it can be carried out by using a suitable catalyst such as platinum catalyst, palladium-carbon, etc., in a suitable solvent under hydrogen stream. Also, it is preferred to add a catalytic amount of an acid in the reductive condensation, and such an acid is exemplified by organic acids such as formic acid, acetic acid, propionic acid, methane sulfonic acid, etc., inorganic acids such as hydrochloric acid, nitric acid, sulfuric acid, etc. This reaction can be suitably carried out under cooling to under heating, preferably at 0° C. to 100° C., more preferably at 10° C. to 50° C.
(Method d): When the objective Compound [I] in which the group R¹ in the formula [I] is a substituted carbonyl group is a compound having a urea bond, it can be prepared by reacting a corresponding Compound [I] in which the group R¹ is hydrogen atom with a corresponding amine compound by using a urea bond forming agent. As the urea bond forming agent, 1,1′-carbonyldiimidazole, phosgene, etc., are preferred, and, for example, 1,1′-carbonyldiimidazole, carbonyl dihalides such as triphosgene and phosgene can be used. This reaction can be carried out, for example, at 0° C. to 80° C., preferably at 0° C. to 50° C. Also, this reaction can be carried out according to the method disclosed in Japanese Unexamined Patent Publication No. Hei. 10-195037.
(Method e): The objective Compound [I] in which the sulfur atom which is a substituent of the group R¹ in the formula [I] is a group containing a group substituted by two oxo groups (for example, sulfonyl group, etc.) can be prepared by treating a corresponding Compound [I] in which the group R¹ is a group having thio group with an oxidizing agent (for example, 3-chloroperbenzoic acid, peracetic acid, sodium periodate, OXONE, etc.). This reaction suitably proceeds, for example, at −80° C. to 150° C., particularly preferably at 0° C. to 40° C.

Also, the objective Compound [I] in which the group R¹ is a substituted sulfonyl group can be prepared by reacting a corresponding Compound [I] in which the group R¹ is a hydrogen atom with a halogenosulfonyl compound which is a corresponding compound in the presence of a base. As the base, triethylamine, etc., can be used. Moreover, this reaction can be carried out, for example, at 0° C. to 50° C.

(Method f): The objective Compound [I] in which the group R¹ in the formula [I] contains amino group can be prepared by removing a protective group from a corresponding Compound [I] in which the group R¹ is a protected amino group. Removal of the protective group can be carried out by the conventional manner (for example, acid treatment, base treatment, catalytic reduction, etc.). Among the present reactions, for example, a reaction by the acid treatment can be carried out at 5° C. to 120° C., a reaction by the base treatment can be carried out at 5° C. to 40° C., and a reaction by the catalytic reduction can be carried out at 10° C. to 40° C.

Also, the objective Compound [I] in which the group R¹ in the formula [I] contains amino group can be prepared by reducing a corresponding Compound [I] in which the group R¹ contains nitro group. Reduction can be carried out in the presence of an acid by reacting with tin dichloride, zinc, etc. This reaction can be carried out, for example, by refluxing the solvent.

Moreover, the objective Compound [I] in which the group R¹ in the formula [I] contains amino group can be prepared by subjecting a corresponding Compound [I] in which the group R¹ contains carboxyl group to Curtius rearrangement, etc. Curtius rearrangement can be carried out, for example, by the method described in Advanced Organic Chemistry, 4th Edition, p. 1054. That is, it can be carried out by converting a carboxyl group into an acid chloride by thionyl chloride, etc., and subsequently subjecting the same to azidation by sodium azide, etc., followed by hydrolysis.

(Method g): The objective Compound [I] in which the group R¹ in the formula [I] contains a substituted carbonylamino group can be prepared by reacting a corresponding compound in which the group R¹ contains amino group with a corresponding carboxylic acid compound or its active ester in the presence or in the absence of a condensing agent. As the condensing agent, 1,1′-carbonyldiimidazole, 1,3-dicyclohexylcarbodiimide, 1-ethyl-3-(3-dimethylaminopropyl)carbodiimide hydrochloride, isobutyl chloroformate or N-methylmorpholine, etc., can be used, which are compounds normally used in a reaction to form an amide bond from a carboxylic acid and an amine. As the active ester of the carboxylic acid compound, an ester with N-hydroxysuccinic imide, N-hydroxyphthalimide, 1-hydroxybenzotriazole or p-nitrophenol can be used. This reaction can be carried out, for example, at −20° C. to 50° C.
(Method h): When a carbon number of the group R¹ in the formula [I] of the objective Compound [I] is to be increased, it can be carried out by Grignard reaction. For example, it can be carried out by reacting with a Grignard reagent such as a corresponding alkyl magnesium chloride, etc. This reaction can be carried out at −50° C. to 0° C.
(Method i): The objective Compound [I] in which the group R¹ in the formula [I] is amino group having a substituent can be prepared by substituting a corresponding compound in which the group R¹ contains amino group with a substituent (for example, an alkoxycarbonyl group such as tert-butoxycarbonyl group, etc., an arylalkoxycarbonyl group such as benzyloxycarbonyl group, etc., an alkanoyl group such as formyl group, acetyl group, propionyl group, etc., an alkyl group such as methyl group, ethyl group, propyl group, etc., an alkylsulfonyl group such as methanesulfonyl group, ethanesulfonyl group, etc., an alkenylsulfonyl group such as vinylsulfonyl group, etc., heterocyclic group such as pyridyl group, etc.) of the amino group by the conventional manner, or reacting with, for example, an alkoxyalkyl alcohol, etc. by using a reagent for synthesizing a carbamate such as N,N′-succinimidylcarbonate, etc. Substitution can be suitably carried out depending on the kind of the substituent by the conventional manner such as alkylation, acylation, sulfonylarion, allylation, etc. Moreover, by substituting hydrogen atom of the amino group with a substituent, a di-substituted product can be prepared. This reaction can be carried out at −20° C. to 50° C.
(Method j): The objective Compound [I] in which the group R¹ in the formula [I] contains free carboxyl group can be prepared by subjecting a corresponding Compound [I] in with the group R¹ containing an esterified carboxyl group to deesterification (for example, depending on the kind of an ester residue, hydrolysis using a base such as sodium hydroxide, etc.; acid treatment by using trifluoroacetic acid, hydrogen chloride, hydrogen bromide, etc., reduction using palladium (black), palladium carbon, etc., under hydrogen atmosphere, and the like) according to the conventional manner. Among the present deesterifications, hydrolysis using a base can be carried out, for example, at 5° C. to 70° C., acid treatment at 5° C. to 8° C., and reduction at 10° C. to 40° C.
(Method k): The objective Compound [I] in which the group R¹ in the formula [I] contains an amide bond can be prepared by reacting a corresponding Compound [I] in which the group R¹ contains free carboxyl group or a corresponding Compound [I] in which the R¹ contains a carboxylic acid ester group with a corresponding amine compound, or reacting a corresponding Compound [I] in which the group R¹ contains free amino group with a corresponding carboxylic acid compound in the presence or in the absence of a condensing agent. As the condensing agent, 1,1′-carbonyldiimidazole, 1,3-dicyclohexylcarbodiimide, 1-ethyl-3-(3-dimethylaminopropyl)carbodiimide hydrochloride, isobutyl chloroformate or N-methylmorpholine, etc., can be used, which are compounds normally used in a reaction to form an amide bond from a carboxylic acid and an amine. This reaction can be carried out, for example, at −20° C. to 50° C.
(Method l): The objective Compound [I] in which the group R¹ in the formula [I] contains hydroxyl group can be prepared by removing a protective group from a corresponding Compound [I] in which the group R¹ contains a protected hydroxyl group by a conventional manner. Removal of the protective group can be carried out depending on the kind of the protective group by an acid treatment, a base treatment, catalytic reduction, etc. This reaction suitably proceeds, for example, at 0° C. to 80° C., particularly at 5° C. to 50° C.

Also, the objective Compound [I] in which the group R¹ in the formula [I] contains hydroxyl group can be prepared by reducing a corresponding Compound [I] in which the group R¹ contains formyl group. Reduction can be carried out by treating the above compound in the presence of a reducing agent such as sodium borohydride, etc. This reaction suitably proceeds, for example, at −80° C. to 80° C., particularly preferably at −70° C. to 20° C.

Moreover, the objective Compound [I] in which the group R¹ in the formula [I] contains hydroxyl group can be prepared by reducing a corresponding Compound [I] in which the group R¹ contains an ester or carboxyl group. Reduction can be carried out by treating the above compound in the presence of a reducing agent such as lithium aluminum hydride, etc. This reaction suitably proceeds, for example, at −50° C. to 200° C., particularly preferably at −20° C. to 60° C.

(Method m): The objective Compound [I] in which the group R¹ in the formula [I] contains a group where the sulfur atom as a substituent is substituted by an oxo group (for example, sulfinyl group, etc.) can be prepared by treating a corresponding Compound [I] in which the group R¹ is a group containing thio group with an oxidizing agent (for example, 3-chloroperbenzoic acid, peracetic acid, sodium periodate, OXONE, etc.). This reaction suitably proceeds, for example, at −80° C. to 150° C., particularly preferably at 0° C. to 40° C.

Also, the objective Compound [I] in which the group R¹ in the formula [I] contains a group where the sulfur atom as a substituent is substituted by two oxo groups (for example, sulfonyl group, etc.) can be prepared by treating a corresponding Compound [I] in which the group R¹ contains thio group with an oxidizing agent (for example, 3-chloroperbenzoic acid, peracetic acid, sodium periodate, OXONE, etc.). This reaction suitably proceeds, for example, at −80° C. to 150° C., particularly preferably at 0° C. to 40° C.

(Method n): The objective Compound [I] in which the group R¹ in the formula [I] is amino group can be prepared by reacting a corresponding Compound [I] in which the group R¹ is hydrogen atom with an aminating agent (for example, tert-butyl nitrite, etc.). This reaction can be carried out, for example, at room temperature to under reflux.

Also, it can be prepared by removing a protective group from a corresponding compound in which the group R¹ is a substituted amino group by a conventional manner.

(Method o): The objective Compound [I] in which the group R¹ in the formula [I] is a cyclized group (for example, oxopyrrolidinyl group, oxo-oxazolidinyl group, etc.) can be prepared by subjecting a corresponding Compound [I] to cyclization. This reaction suitably proceeds at −50° C. to 200° C., particularly preferably at −20° C. to 60° C.
(Method p): The objective Compound [I] in which the group R¹ in the formula [I] is an optionally substituted alkyl group can be prepared by alkylating a corresponding Compound [I] in which the group R¹ in the formula [I] is hydrogen by a conventional manner. This reaction proceeds at 20° C. to 80° C.

The solvent to be used in the reactions described in the above-mentioned (Method a) to (Method p) is not specifically limited so long as it does not inhibit the reaction, and, for example, dioxane, ethylene glycol dimethyl ether, dimethylacetamide, dimethylformamide, hexamethylphosphoramide, benzene, tetrahydrofuran, toluene, ethyl acetate, alcohol, dichloromethane, carbon tetrachloride, 1,3-dimethyl-2-imidazolidine, acetic acid, diethyl ether, methoxyethane, dimethylsulfoxide, acetonitrile, water or a mixed solvent of the above solvents can be used by optionally selecting them.

Incidentally, the starting Compound [II] of the present invention is a novel compound, and can be prepared, for example, by the following chemical reaction formulae.

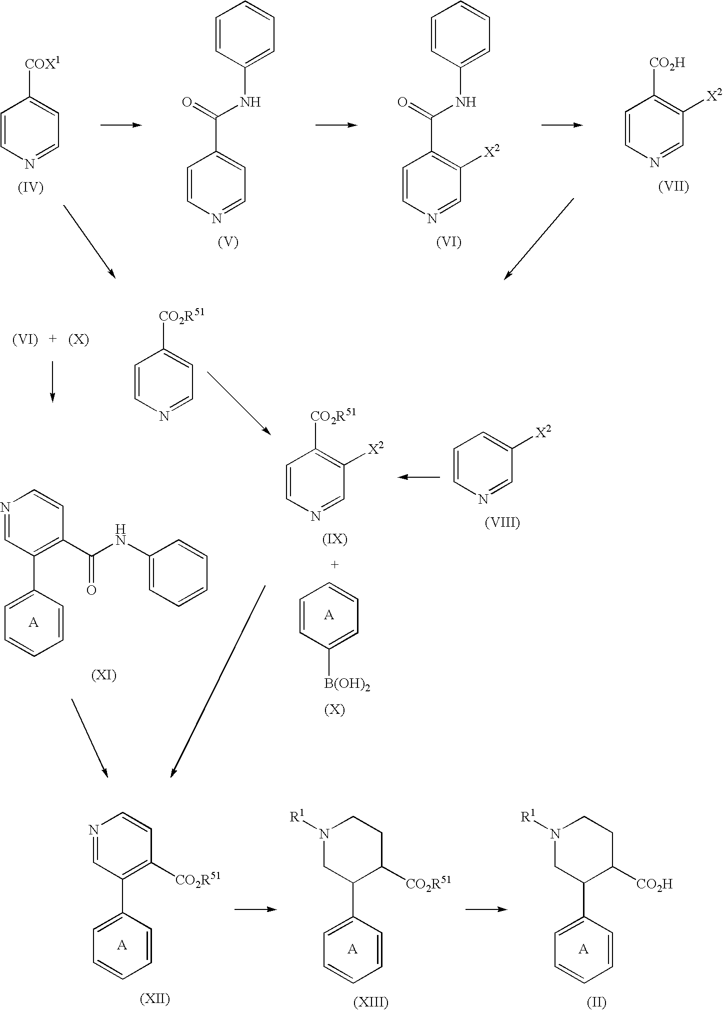

wherein R⁵¹ represents an alkyl group, X¹ represents a leaving group, X² represents a leaving group, Ring A and R¹ have the same meanings as defined above.

That is, the pyridine compound [IV] is subjected to condensation with aniline to give Compound [V], then, subjecting to halogenation to give Compound [VI], and the aniline is eliminated, and esterifying the acyl group of the obtained compound to give Compound [VII]. Also, Compound [IX] is obtained by esterifying the carboxyl group of Compound [VII], subjecting Compound [VIII] to C—C bond formation, or esterifying the acyl group of Compound [IV] and then to haloganate. The obtained Compound [IX] and Compound [X] are coupled or Compound [VI] and Compound [X] are coupled to give Compound [XI], and the aniline is eliminated to give Compound [XII], the resulting Compound [XII] is subjected to reduction, then, a substituent of the amino group is introduced to give Compound [XIII]. An ester group of the resulting Compound [XIII] is converted to a carboxyl group to give Compound [II].

Compound [II] has an asymmetric carbon, and optical isomers exist based on the asymmetric carbon. For example, when cis isomer and trans isomer are obtained as a mixture, the respective cis isomer and trans isomer can be obtained separately by a conventional manner such as silica gel chromatography, etc. Also, optical isomers of Compound [II] can be obtained, for example, by optically resolving racemic mixtures of Compound [XIII] where R¹ is hydrogen atom or Compound [II] according to a conventional manner.

In the case of a compound wherein R¹ of Compound [XIII] is hydrogen atom, optical resolution can be carried out, for example, by acting Compound [XIII] with N-acyl-optically active amino acid, N-sulfonyl-optically active amino acid or optically active carboxylic acid, and separating and collecting one of the diastereomer salts utilizing the differences in solubility between two kinds of the formed diastereomer salts. The acyl group of the N-acyl-optically active amino acid can be exemplified by, for example, acetyl group, propionyl group or benzyloxycarbonyl group, and the sulfonyl group of the N-sulfonyl-optically active amino acid can be exemplified by, for example, tosyl group or mesyl group, and the optically active amino acid can be exemplified by, for example, L-phenylalanine, L-leucine, L-glutamine, L-methionine, L-valine, L-threonine, D-phenylalanine or D-phenylglycine. Also, the optically active carboxylic acid is exemplified by mandelic acid, malic acid or tartaric acid derivatives. The tartaric acid derivatives are exemplified by dibenzoyl-L-tartaric acid, di-ptoluoyl-L-tartaric acid, dibenzoyl-D-tartaric acid, di-p-toluoyl-D-tartaric acid, etc.

Also, in the case of Compound [II], optical resolution can be carried out by, for example, acting Compound [II] with O-alkyl-optically active amino acid or an optically active amine derivative, and separating and collecting one of the diastereomer salts utilizing the differences in solubility between two kinds of the formed diastereomer salts. The optically active amino acid can be exemplified by, for example, L-phenylalanine, L-leucine, L-glutamine, L-methionine, L-valine, L-threonine, D-phenylalanine or D-phenylglycine. The alkyl group of the O-alkyl-optically active amino acid can be exemplified by methyl group, ethyl group, etc. The optically active amine derivative can be exemplified by brucine, quinidine, (S)-1-phenethylamine, (R)-1-phenethylamine, (R)-(−)-1-cyclohexylethylamine, (S)-(+)-1-cyclohexylethylamine, etc.

Further, in preparation of the objective compound or the starting materials of the present invention, when the starting materials or the intermediates have a functional group, suitable protecting groups can be introduced to each of the functional group by a conventional method, besides the above described method, and if they are not necessary, these protecting groups can be suitably removed.

For example, in the present specification, as the protective group for the amino group, a protective group to be generally used for protecting the amino group for applying the same to a reaction, and it can be specifically exemplified by, for example, an alkoxycarbonyl group such as tert-butoxycarbonyl group, an arylalkoxycarbonyl group such as benzyloxycarbonyl group, etc.

In the present specification, the alkyl group means, for example, a straight or branched alkyl group having 1 to 6 carbon atoms such as methyl group, ethyl group, propyl group, isopropyl group, butyl group, isobutyl group, tert-butyl group, isopentyl group, etc., preferably those having 1 to 4 carbon atoms. The alkenyl group means, for example, a straight or branched alkenyl group having 2 to 7 carbon atoms such as vinyl group, allyl group, propenyl group, isopropenyl group, etc., preferably those having 2 to 5 carbon atoms. The alkoxy group means a straight or branched alkoxy group having 1 to 6 carbon atoms such as methoxy group, ethoxy group, propoxy group, isopropoxy group, butoxy group, etc., preferably those having 1 to 4 carbon atoms. The alkanoyl group means a straight or branched alkanoyl group having 1 to 6 carbon atoms such as formyl group, acetyl group, propionyl group, butyryl group, valeryl group, tert-butylcarbonyl group, etc., preferably those having 1 to 4 carbon atoms. The alkylene group means, for example, a straight or branched alkylene group having 2 to 7 carbon atoms such as methylene group, ethylene group, propylene group, butylene group, etc., preferably those having 2 to 5 carbon atoms. The cycloalkyl group means, for example, a cycloalkyl group having 3 to 8 carbon atoms such as cyclopropyl group, cyclobutyl group, cyclopentyl group, cyclohexyl group, cycloheptyl group, cyclooctyl group, etc., preferably those having 3 to 6 carbon atoms. Further, the halogen atom is exemplified by chlorine atom, bromine atom, fluorine atom and iodine atom.

### EXAMPLE

### Example 1

To 50 ml of a N,N-dimethylformamide solution containing 2.7 g of 1-tert-butoxycarbonyl-4-carboxyl-3-(4-fluoro-2-methylphenyl)piperidine and 2.72 g of (R)-1-(3,5-bistrifluoromethylphenyl)ethyl-1-methylamine were added 1.92 g of 1-[3-(dimethylamino)propyl]-3-ethylcarbodiimide hydrochloride and 1.53 g of 1-hydroxybenzotriazole, and the mixture was stirred at room temperature for 16 hours. To the reaction mixture were added ethyl acetate and brine, the liquids were separated, and the obtained organic layer was washed with an aqueous sodium hydrogen bicarbonate solution and water. The obtained organic layer was dried over anhydrous magnesium sulfate, and concentrated under reduced pressure. The obtained residue was purified by silica gel column chromatography (n-hexane:ethyl acetate=3:1) to give 730 mg of (a) (3S,4S)-1-tert-butoxycarbonyl-4-[N-{1-(R)-(3,5-bistrifluoromethylphenyl)ethyl}-N-methyl]aminocarbonyl-3-(4-fluoro-2-methylphenyl)piperidine and 680 mg of (b) (3S,4R)-1-tert-butoxycarbonyl-4-[N-{1-(R)-(3,5-bistrifluoromethylphenyl)ethyl}-N-methyl]aminocarbonyl-3-(4-fluoro-2-methylphenyl)piperidine shown in Table 1 below.

### Examples 2 to 5

The corresponding starting materials were used and treated in the same manner as in Example 1, to give compounds as shown in Tables 1 to 3 below.

### Example 6

To 1.2 g of trans-1-tert-butoxycarbonyl-4-{N-(3,5-bistrifluoromethylbenzyl)-N-methyl}aminocarbonyl-3-(4-fluoro-2-methylphenyl)piperidine was added 10 ml of an ethyl acetate solution containing 4M hydrochloric acid, and the mixture was stirred for 1 hour and then concentrated under reduced pressure. To the residue were added an aqueous 4M sodium carbonate solution and ethyl acetate, and the liquids were separated. The organic layer was dried over anhydrous sodium sulfate, and concentrated under reduced pressure to give 600 mg of trans-4-{N-(3,5-bistrifluoromethylbenzyl)-N-methyl}aminocarbonyl-3-(4-fluoro-2-methylphenyl)piperidine shown in Table 4 below.

### Examples 7 to 12

The corresponding starting materials were used and treated in the same manner as in Example 6, to give compounds as shown in Table 4 below.

### Example 13

To 3 ml of N,N-dimethylformamide solution containing 90 mg of trans-4-{N-(3,5-bistrifluoromethylbenzyl)-N-methyl}aminocarbonyl-3-(4-fluoro-2-methylphenyl)piperidine and 24 mg of 3-hydroxy-3-methylbutanoic acid were added 40 mg of 1-[3-(dimethylamino)propyl]-3-ethylcarbodiimide hydrochloride and 31 mg of 1-hydroxybenzotriazole, and the mixture was stirred at room temperature for 16 hours. To the reaction mixture were added ethyl acetate and brine, liquids were separated, and the organic layer was successively washed with an aqueous sodium hydrogen bicarbonate solution and water. The obtained organic layer was dried over anhydrous magnesium sulfate, and concentrated under reduced pressure. The obtained residue was purified by silica gel column chromatography (chloroform:methanol=19:1) to give 75 mg of trans-4-{N-(3,5-bistrifluoromethylbenzyl)-N-methyl}aminocarbonyl-3-(4-fluoro-2-methylphenyl)-1-(3-hydroxy-3-methylbutyryl)piperidine shown in Table 5 below.

### Examples 14 to 26

The corresponding starting materials were used and treated in the same manner as in Example 13, to give compounds as shown in Tables 5 to 7 below.

### Example 27

To 3 ml of a dichloromethane solution containing 143 mg of trans-4-{N-(3,5-bistrifluoromethylbenzyl)-N-methyl}aminocarbonyl-3-(4-fluoro-2-methylphenyl)piperidine, 45 mg of 1-acetyl-4-piperidone and 0.01 ml of acetic acid was added 110 mg of sodium triacetoxyborohydride, and the mixture was stirred at room temperature for 16 hours. To the reaction mixture was added sodium carbonate, the resulting mixture was stirred for 1 hour, chloroform was added to the mixture, the liquids were separated, and the aqueous layer was extracted again with chloroform. The combined organic layers were dried over anhydrous magnesium sulfate, and concentrated under reduced pressure. The obtained residue was purified by silica gel column chromatography (chloroform:methanol=19:1) to give 120 mg of trans-1-(1-acetoxypiperidin-4-yl)-4-{N-(3,5-bistrifluoromethylbenzyl)-N-methyl}aminocarbonyl-3-(4-fluoro-2-methylphenyl)piperidine shown in Table 8 below.

### Examples 28 to 31

The corresponding starting materials were used and treated in the same manner as in Example 27, to give compounds as shown in Table 8 below.

### Example 32

To 2.5 ml of a tetrahydrofuran solution containing 145 mg of trans-4-{N-(3,5-bistrifluoromethylbenzyl)-N-methyl}aminocarbonyl-3-(4-fluoro-2-methylphenyl)piperidine was added 42 mg of 1,1′-carbonyldiimidazole, and the mixture was stirred at 50° C. for 1 hour. After the reaction mixture was concentrated, 5 ml of acetonitrile and 0.5 ml of methyl iodide were successively added to the residue and the mixture was stirred at 70° C. for 1 hour. The reaction mixture was concentrated again, the concentrate was dissolved in 5 ml of tetrahydrofuran, 120 mg of 2-aminoethanol and 0.04 ml of triethylamine were added to the mixture, and the resulting mixture was stirred at 40° C. for 16 hours. Water and ethyl acetate were added to the reaction mixture, the liquids were separated, and the organic layer was washed with water twice, dried over anhydrous magnesium sulfate and concentrated under reduced pressure. The obtained residue was purified by silica gel column chromatography (chloroform:methanol=19:1) to give 103 mg of trans-4-{N-(3,5-bistrifluoromethylbenzyl)-N-methyl}aminocarbonyl-3-(4-fluoro-2-methylphenyl)-1-{(2-hydroxyethyl)aminocarbonyl}piperidine shown in Table 9 below.

### Examples 33 and 34

The corresponding starting materials were used and treated in the same manner as in Example 32, to give compounds as shown in Table 9 below.

### Example 35

To 4 ml of a tetrahydrofuran solution containing 100 mg of trans-4-{N-(3,5-bistrifluoromethylbenzyl)-N-methyl}aminocarbonyl-3-(4-fluoro-2-methylphenyl)piperidine were added 0.3 ml of triethylamine and 0.16 ml of methanesulfonyl chloride, and the mixture was stirred at room temperature for 16 hours. After completion of the reaction, water and ethyl acetate were added to the mixture, liquids were separated and the organic layer was washed with water. The obtained organic layer was dried over anhydrous magnesium sulfate, and concentrated under reduced pressure. The obtained residue was purified by silica gel column chromatography (chloroform:methanol=19:1) to give 93 mg of trans-4-{N-(3,5-bistrifluoromethylbenzyl)-N-methyl}aminocarbonyl-3-(4-fluoro-2-methylphenyl)-1-methanesulfonylpiperidine shown in Table 9 below.

### Example 36

The corresponding starting materials were used and treated in the same manner as in Example 1, to give compounds as shown in Table 10 below.

### Example 37

(1) To 80 ml of a tetrahydrofuran solution containing 0.80 g of (3S,4S)-1-benzyloxycarbonyl-4-carboxyl-3-(4-fluoro-2-methylphenyl)piperidine were added a catalytic amount of N,N-dimethylformamide and 0.56 g of thionyl chloride, and the mixture was stirred at room temperature for 16 hours, then, the reaction mixture was concentrated under reduced pressure. A solution of the obtained residue dissolved in 20 ml of dichloromethane was cooled to 0° C., 0.26 g of triethylamine and 0.94 g of N-{1-(3,5-bistrifluoromethylphenyl)-1-methyl-ethyl}-N-methylamine were added thereto, and the resulting mixture was stirred at room temperature for 2 days. Chloroform and water were added to the reaction mixture, and the liquids were separated. The organic layer was washed successively with a saturated aqueous citric acid solution and brine, dried over anhydrous sodium sulfate, and concentrated under reduced pressure. The obtained residue was purified by basic silica gel column chromatography (hexane:ethyl acetate=19:1→2:1) to give 0.17 g of (3S,4S)-1-benzyloxycarbonyl-4-[N-{1-(3,5-bistrifluoromethylphenyl)-1-methylethyl}-N-methyl]aminocarbonyl-3-(4-fluoro-2-methylphenyl)piperidine shown in Table 10 below.
(2) To 3 ml of a methanol solution containing 0.16 g of the compound obtained in the above-mentioned (1) was added 25 mg of 10% palladium carbon, the mixture was stirred under hydrogen atmosphere at room temperature for 16 hours, and further stirred after adding 20 mg of palladium hydroxide and 0.2 ml of 6M aqueous hydrochloric acid solution under hydrogen atmosphere at room temperature for 3 hours. The reaction mixture was filtered through a membrane filter and the filtrate was concentrated under reduced pressure to give 0.11 g of (3S,4S)-4-(N-{1-(3,5-bistrifluoromethylphenyl)-1-methylethyl)-N-methyl}aminocarbonyl-3-(4-fluoro-2-methylphenyl)piperidine shown in Table 10 below.

### Examples 38 to 103

The corresponding starting materials were used and treated in the same manner as in Example 13, to give compounds as shown in Tables 11 to 19 below.

### Example 104

(1) To 5 ml of a N,N-dimethylformamide solution containing 400 mg of (3S,4S)-4-{N-1-(S)-(3,5-bistrifluoromethylphenyl)ethyl-N-methyl}aminocarbonyl-3-(4-fluoro-2-methylphenyl)piperidine and 180 mg of 1-tert-butoxycarbonyl-3-azetidine carboxylic acid were added 180 mg of 1-hydroxybenztriazole and 224 mg of 1-{3-(N,N-dimethylamino)propyl}-3-ethylcarbodiimide hydrochloride, and the mixture was stirred at room temperature overnight. To the reaction mixture was added an aqueous sodium hydrogen carbonate solution, ethyl acetate was added and the liquids were separated. The organic layer was washed with water and brine, dried and concentrated under reduced pressure. The obtained residue was purified by silica gel column chromatography (hexane:ethyl acetate=100:0→50:50) to give 539 mg of (3S,4S)-1-{(1-tert-butoxycarbonylazetidin-3-yl)carbonyl}-4-{N-1-(S)-(3,5-bistrifluoromethylphenyl)ethyl-N-methyl}aminocarbonyl-3-(4-fluoro-2-methylphenyl)piperidine.
(2) To 2 ml of an ethyl acetate solution containing 539 mg of the compound obtained in the above-mentioned (1) was added 6 ml of an ethyl acetate solution containing 4M hydrogen chloride, the mixture was stirred at room temperature overnight, and the reaction mixture was concentrated under reduced pressure. The obtained residue was purified by silica gel thin-layer chromatography (chloroform:methanol=9:1) to give 195 mg of (3S,4S)-1-{(azetidin-3-yl)carbonyl}-4-{N-1-(S)-(3,5-bistrifluoromethylphenyl)ethyl-N-methyl}aminocarbonyl-3-(4-fluoro-2-methylphenyl)piperidine.
(3) To 1.5 ml of a dichloromethane solution containing 36 mg of the compound obtained in the above-mentioned (2) was added 11 μl of triethylamine at room temperature, and after adding dropwise 75 μl of a tetrahydrofuran solution containing 1.0M acetyl chloride at 0° C., the resulting mixture was stirred at room temperature overnight. The reaction mixture was filtered, and concentrated under reduced pressure. The residue was purified by silica gel thin-layer chromatography (chloroform:methanol=9:1) to give 27 mg of (3S,4S)-1-{(1-acetylazetidin-3-yl)carbonyl}-4-{N-1-(S)-(3,5-bistrifluoromethylphenyl)ethyl-N-methyl}aminocarbonyl-3-(4-fluoro-2-methylphenyl)piperidine shown in Table 19 below.

### Examples 105 to 115

The corresponding starting materials were used and treated in the same manner as in Example 104, to give compounds as shown in Tables 19 to 21 below.

### Example 116

A solution of 3 ml of tetrahydrofuran containing 100 mg of the compound obtained in Example 55 was cooled to −20° C., then 1 ml of a tetrahydrofuran solution containing 1.0M methyl magnesium chloride was added dropwise to the solution, and the resulting mixture was stirred for 1 hour. An aqueous ammonium chloride solution and ethyl acetate were added to the mixture, the liquids were separated, and the organic layer was washed with brine. The obtained organic layer was dried over anhydrous magnesium sulfate, and concentrated under reduced pressure. The residue was purified by silica gel column chromatography (chloroform:methanol-19:1) to give 100 mg of (3S,4S)-4-{N-1-(R)-(3,5-bistrifluoromethylphenyl)ethyl-N-methyl}aminocarbonyl-3-(4-fluoro-2-methylphenyl)-1-(4-hydroxy-4-methylpentanoyl)piperidine shown in Table 22 below.

### Example 117

The corresponding starting materials were used and treated in the same manner as in Example 116, to give compounds as shown in Table 22 below.

### Example 118

The same treatment was carried out as in Example 13 by using (3S,4S)-4-{N-1-(R)-(3,5-bistrifluoromethylphenyl)ethyl-N-methyl}-aminocarbonyl-3-(4-fluoro-2-methylphenyl)piperidine, and 2-tertbutoxycarbonylamino-2-methylpropionic acid, then, the resulting material was treated with a 4M hydrochloric acid-ethyl acetate solution to tive (3S,4S)-1-(2-amino-2-methylpropionyl)-4-{N—(R)-2-(3,5-bistrifluoromethylphenyl)ethyl-N-methyl}aminocarbonyl-3-(4-fluoro-2-methylphenyl)piperidine shown in Table 22 below.

### Example 119

To 2 ml of a N,N-dimethylformamide solution containing 60 mg of the compound obtained in Example 87 were added 10 mg of sodium hydride (40% in oil) and 0.05 ml of methyl iodide at 0° C., and the mixture was stirred for 2 hours. After completion of the stirring, brine and ethyl acetate were added to the mixture, the liquids were separated and the organic layer was washed with brine. The organic layer was dried over anhydrous magnesium sulfate, and concentrated under reduced pressure. The obtained residue was purified by silica gel column chromatography (chloroform:methanol=19:1) to give 45 mg of (3S,4S)-1-(azetidin-3-yl)carbonyl-4-{N-1-(R)-(3,5-bistrifluoromethylphenyl)ethyl-N-methyl}aminocarbonyl-3-(4-fluoro-2-methylphenyl)piperidine shown in Table 22 below.

### Examples 120 to 124

The corresponding starting materials were used and treated in the same manner as in Example 119, to give compounds as shown in Table 22 below.

### Examples 125 to 159

The corresponding starting materials were used and treated in the same manner as in Example 27, to give compounds as shown in Tables 23 to 26 below.

### Example 160

In 1.5 ml of methanol was dissolved 30 mg of (3S,4S)-4-{N-1-(R)-(3,5-bistrifluoromethylphenyl)ethyl-N-methyl}aminocarbonyl-3-(4-fluoro-2-methylphenyl)piperidine, and 12 μl of acrylonitrile was added to the mixture at room temperature and allowed to stand for 30 minutes. The reaction mixture was purified by silica gel thin-layer chromatography (developed by chloroform:methanol=9:1, eluted with dichloromethane:ethanol:aqueous ammonia=100:10:1), and lyophilized by using tert-butanol to give 27 mg of (3S,4S)-4-{N-1-(R)-(3,5-bistrifluoromethylphenyl)ethyl-N-methyl}aminocarbonyl-1-(2-cyanoethyl)-2-(4-fluoro-2-methylphenyl)piperidine shown in Table 26 below.

### Examples 161-170

The corresponding starting materials were used and treated in the same manner as in Example 160, to give compounds as shown in Tables 26 and 27 below.

### Example 171

To 1 ml of a N,N-dimethylformamide solution containing 49 mg of (3S,4S)-4-{N-1-(R)-(3,5-bistrifluoromethylphenyl)ethyl-Nmethyl}aminocarbonyl-3-(4-fluoro-2-methylphenyl)piperidine were added 0.028 ml of triethylamine and 0.012 ml of 2-fluorobromoethane at room temperature, and the mixture was stirred overnight. Ethyl acetate and water were added to the reaction mixture, liquids were separated, and the organic layer was washed with brine. The organic layer was dried over ahydrous magnesium sulfate, and concentrated under reduced pressure. The obtained residue was purified by basic silica gel thin-layer chromatography (n-hexane:ethyl acetate=2:1) to give 42 mg of (3S,4S)-4-{N-1-(R)-(3,5-bistrifluoromethylphenyl)ethyl-N-methyl}aminocarbonyl-1-(2-fluoroethyl)-3-(4-fluoro-2-methylphenyl)piperidine shown in Table 27 below.

### Examples 172 to 194

The corresponding starting materials were used and treated in the same manner as in Example 171, to give compounds as shown in Tables 27 to 29 below.

### Example 195

To 56 mg of the compound obtained in Example 179 was added 2 ml of a methanol solution containing 0.5M potassium hydroxide at room temperature and the mixture was stirred overnight. The reaction mixture was neutralized by a saturated aqueous citric acid solution and an aqueous sodium hydroxide solution, chloroform was added to the mixture and the liquids were separated. The organic layer was dried over anhydrous magnesium sulfate, and concentrated under reduced pressure to give 48 mg of (3S,4S)-4-{N-1-(R)-(3,5-bistrifluoromethylphenyl)ethyl-N-methyl}aminocarbonyl-1-carboxymethyl-3-(4-fluoro-2-methylphenyl)piperidine shown in Table 29 below.

### Example 196

The corresponding starting materials were used and treated in the same manner as in Example 195, to give compounds as shown in Table 29 below.

### Example 197

In 1 ml of acetonitrile was dissolved 40 mg of (3S,4S)-4-{N-1-(R)-(3,5-bistrifluoromethylphenyl)ethyl-N-methyl}aminocarbonyl-3-(4-fluoro-2-methylphenyl)piperidine, a mixture to which 15 mg of 2-chloromethylpyridine N-oxide hydrochloride and 43 μl of diisopropylethylamine were added to the solution and the resulting mixture was stirred at 80° C. for 4 hours. After cooling the reaction mixture to room temperature, it was purified by silica gel thin-layer chromatography (developed by and eluted with dichloromethane:ethanol:aqueous ammonia=100:10:1), and lyophilized by using tert-butanol, to give 26 mg of (3S,4S)-4-{N-1-(R)-(3,5-bistrifluoromethylphenyl)ethyl-N-methyl}aminocarbonyl-2-(4-fluoro-2-methylphenyl)-1-(1-oxypyridin-2-yl)methylpiperidine shown in Table 30 below.

### Examples 198 to 202

The corresponding starting materials were used and treated in the same manner as in Example 197, to give compounds as shown in Table 30 below.

### Example 203

To 2 ml of an acetonitrile solution containing 50 mg of (3S,4S)-4-{N-1-(R)-(3,5-bistrifluoromethylphenyl)ethyl-N-methyl}-aminocarbonyl-3-(4-fluoro-2-methylphenyl)piperidine were added 32 mg of methyl 2-(toluene-4-sulfonyloxy)propionate and 50 mg of potassium carbonate, and the mixture was stirred under reflux for 2 hours. After completion of the stirring, ethyl acetate and water were added to the mixture, liquids were separated, and the organic layer was concentrated under reduced pressure. The obtained residue was purified by silica gel column chromatography (chloroform:methanol=19:1) to give 36 mg of methyl (3S,4S)-2-[4-{N—(R)(3,5-bistrifluoromethylphenyl)ethyl-N-methyl}aminocarbonyl-3-(4-fluoro-2-methylphenyl)piperidine-1-yl]propionate shown in Table 30 below.

### Example 204

The corresponding starting materials were used and treated in the same manner as in Example 203, to give compounds as shown in Table 30 below.

### Example 205

To 3 ml of an acetonitrile solution containing 100 mg of (3S,4S)-4-{N-1-(S)-(3,5-bistrifluoromethylphenyl)ethyl-N-methyl}-aminocarbonyl-3-(4-fluoro-2-methylphenyl)piperidine were added 67.2 mg of 2-(tert-butoxycarbonylamino)ethyl bromide and 55.2 mg of potassium carbonate at room temperature, and the mixture was stirred at 80° C. overnight. To the reaction mixture were added 3 ml of N,N-dimethylformamide and 50 mg of 2-(tert-butoxycarbonylamino)ethyl bromide, and the mixture was further stirred at 110° C. overnight. To the reaction mixture was added an aqueous ammonium chloride solution, and the liquids were separated by adding ethyl acetate. The organic layer was washed with brine, dried, and concentrated under reduced pressure. The obtained residue was purified by silica gel column chromatography (chloroform:methanol=100:0→91:9). To the resulting compound was added 2 ml of a 4M aqueous hydrochloric acid solution, the resulting mixture was stirred 1 day, and the reaction mixture was concentrated under reduced pressure. The obtained residue was purified by column chromatography (LC-MS) (water-methanol). The obtained compound was dissolved in 2 ml of dichloromethane, 14 μl of triethylamine and 7 μl of acetyl chloride were added dropwise to the solution, and the resulting mixture was stirred for 3 days. To the reaction mixture was added an aqueous sodium hydrogen carbonate solution, the liquids were separated by adding dichloromethane, and the organic layer was concentrated under reduced pressure. The obtained residue was purified by silica gel thin-layer chromatography (chloroform:methanol=9:1) and column chromatography (LC-MS) (water-methanol) to give 3.6 mg of (3S,4S)-1-(2-acetylaminoethyl)-4-{N-1-(S)-(3,5-bistrifluoromethylphenyl)ethyl-N-methyl}aminocarbonyl-3-(4-fluoro-2-methylphenyl)piperidine shown in Table 30 below.

### Example 206

To 2 ml of a methanol solution containing 100 mg of the compound obtained in Example 203 was added 2 ml of a 2M aqueous sodium hydroxide solution, and the mixture was stirred at room temperature for 16 hours. After adding 2.2 ml of a 2M aqueous hydrochloric acid solution, the mixture was extracted with chloroform three times. The organic layer was dried over anhydrous magnesium sulfate, and concentrated under reduced pressure. To 2 ml of a N,N-dimethylformamide solution containing the obtained residue were added 0.1 ml of hydroxyethylamine, 40 mg of 1-[3-(dimethylamino)propyl]-3-ethylcarbodiimide hydrochloride and 31 mg of 1-hydroxybenzotriazole, and the mixture was stirred at 50° C. for 16 hours. After completion of the stirring, ethyl acetate and brine were added, the liquids were separated, and the organic layer was washed twice with an aqueous sodium bicarbonate solution, and concentrated under reduced pressure. The obtained residue was purified by silica gel column chromatography (chloroform:methanol=19:1) to give 64 mg of (3S,4S)-4-{N-1-(R)-(3,5-bistrifluoromethylphenyl)ethyl-N-methyl}aminocarbonyl-3-(4-fluoro-2-methylphenyl)-1-{1-(R)-(2-hydroxyaminocarbonyl)ethyl}piperidine shown in Table 31 below.

### Examples 207 to 211

The corresponding starting materials were used and treated in the same manner as in Example 206, to give compounds as shown in Table 31 below.

### Example 212

(1) To 5 ml of a dichloromethane solution containing 300 mg of (3S,4S)-4-{N-1-(S)-(3,5-bistrifluoromethylphenyl)ethyl-N-methyl}-aminocarbonyl-3-(4-fluoro-2-methylphenyl)piperidine was added 150 mg of 1-benzyloxycarbonylazetidin-3-one, and the mixture was stirred at room temperature for 90 minutes. After adding 86 μl of acetic acid to the reaction mixture, 648 mg of sodium triacetoxyborohydride was added to the same, and the mixture was stirred at room temperature overnight. To the reaction mixture was added an aqueous sodium hydrogen carbonate solution and the liquids were separated by adding chloroform. The organic layer was washed with brine, dried, and concentrated under reduced pressure. The obtained residue was purified by silica gel column chromatography (chloroform:methanol=100:0→95:5) to give 330 mg of (3S,4S)-1-(1-benzyloxycarbonylazetidin-3-yl)-4-{N-1-(S)-(3,5-bistrifluoromethylphenyl)ethyl-N-methyl}aminocarbonyl-3-(4-fluoro-2-methylphenyl)piperidine.
(2) To 5 ml of a methanol solution containing 330 mg of the compound obtained in the above-mentioned (1) was added 70 mg of 10% palladium carbon under nitrogen atmosphere at room temperature, and the mixture was stirred under hydrogen atmosphere for 4 hours. The reaction mixture was filtered and then concentrated. The obtained residue was purified by basic silica gel thin-layer chromatography (chloroform:methanol=19:1) to give 103 mg of (3S,4S)-1-(azetidin-3-yl)-4-{N-1-(S)-(3,5-bistrifluoromethylphenyl)ethyl-N-methyl}aminocarbonyl-3-(4-fluoro-2-methylphenyl)piperidine.
(3) To 1.5 ml of a dichloromethane solution containing 38 mg of the compound obtained in the above-mentioned (2) was added 24 μl of triethylamine at room temperature, and 1.4 ml of a tetrahydrofuran solution containing 0.1M acetyl chloride was added dropwise to the mixture at 0° C. The resulting mixture was stirred at room temperature overnight. The reaction mixture was filtered, and concentrated under reduced pressure. The obtained residue was purified by silica gel thin-layer chromatography (chloroform:methanol=9:1) to give 22 mg of (3S,4S)-1-(1-acetoxyazetidin-3-yl)-4-{N-1-(S)(3,5-bistrifluoromethylphenyl)ethyl-N-methyl}aminocarbonyl-3-(4-fluoro-2-methylphenyl)piperidine shown in Table 31 below.

### Examples 213 to 222

The corresponding starting materials were used and treated in the same manner as in Example 212, to give compounds as shown in Tables 31 to 33 below.

### Example 223

To 2.5 ml of an acetonitrile solution containing 100 mg of (3S,4S)-4-{N-1-(R)-(3,5-bistrifluoromethylphenyl)ethyl-N-methyl}aminocarbonyl-3-(4-fluoro-2-methylphenyl)piperidine and 0.2 ml of 2-(2-bromoethoxy)tetrahydropyran was added 200 mg of potassium carbonate, and the resulting mixture was stirred under reflux for 2 hours. After the reaction mixture was cooled to room temperature, diisopropyl ether was added to the mixture and the mixture was filtered. The filtrate was concentrated under reduced pressure, then 2 ml of a dioxane solution containing 4M hydrochloric acid was added to the filtrate and the resulting mixture was stirred for 2 hours. The reaction mixture was concentrated under reduced pressure, and purified by silica gel column chromatography (chloroform:methanol=19:1) to give 56 mg of (3S,4S)-4-{N-1-(R)-(3,5-bistrifluoromethylphenyl)ethyl-N-methyl}aminocarbonyl-3-(4-fluoro-2-methylphenyl)-1-(2-hydroxyethyl)piperidine shown in Table 33 below.

### Examples 224 to 225

The corresponding starting materials were used and treated in the same manner as in Example 223, to give compounds as shown in Table 33 below.

### Example 226

To 2 ml of a dichloromethane solution containing 65 mg of the compound obtained in Example 152 was added 55 mg of trifluoroacetic acid, and the mixture was cooled to 0° C. To the solution was added 55 mg of metachloroperbenzoic acid, and the mixture was stirred at 0° C. for 1 hour. To the mixture were added an aqueous sodium bicarbonate solution and chloroform, the resulting mixture was stirred, the liquids were separated, and the organic layer was concentrated under reduced pressure. The obtained residue was purified by silica gel column chromatography (chloroform:methanol=19:1) to give 18 mg of (a) (3S,4S)-4-{N—(R)-2-(3,5-bistrifluoromethylphenyl)ethyl-N-methyl}aminocarbonyl-3-(4-fluoro-2-methylphenyl)-1-(1-oxothiethan-3-yl)-piperidine and 36 mg of (b) (3S,4S)-4-{N—(R)-2-(3,5-bistrifluoromethylphenyl)ethyl-N-methyl}aminocarbonyl-3-(4-fluoro-2-methylphenyl)-1-(1,1-dioxothiethan-3-yl)piperidine shown in Table 33 below.

### Example 227

The corresponding starting materials were used and treated in the same manner as in Example 226, to give compounds as shown in Table 33 below.

### Example 228

To 2.5 ml of a dichloromethane solution containing 100 mg of the compound obtained in Example 147 was added 80 mg of methanesulfonic acid, and the mixture was cooled to 0° C. To the solution was added 100 mg of metachloroperbenzoic acid (70-75%), the mixture was stirred at 0° C. for 2 hours, and the mixture was stirred at room temperature for 16 hours. To the mixture were added 4M aqueous sodium carbonate solution and chloroform, the resulting mixture was stirred, the liquids were separated, and the organic layer was concentrated under reduced pressure. The obtained residue was purified by silica gel column chromatography (chloroform:methanol=19:1) to give 55 mg of (3S,4S)-4-{N—(R)-2-(3,5-bistrifluoromethylphenyl)ethyl-N-methyl}aminocarbonyl-1-(1,1-dioxotetrahydrothiopyran-4-yl)-3-(4-fluoro-2-methylphenyl)piperidine shown in Table 33 below.

### Examples 229 to 230

The corresponding starting materials were used and treated in the same manner as in Example 228, to give compounds as shown in Table 34 below.

### Examples 231 to 261

The corresponding starting materials were used and treated in the same manner as in Example 32, to give compounds as shown in Table 34 to Table 38 below.

### Examples 262 to 263

The corresponding starting materials were used and treated in the same manner as in Example 226, to give compounds as shown in Table 38 below.

### Example 264

3 ml of an acetonitrile solution containing 150 mg of (3S,4S)-4-{N-1-(R)-(3,5-bistrifluoromethylphenyl)ethyl-N-methyl}-aminocarbonyl-3-(4-fluoro-2-methylphenyl)piperidine and 50 mg of 2-chloropyrazine was stirred under reflux for 16 hours. The reaction mixture was cooled to room temperature, an aqueous sodium bicarbonate solution and ethyl acetate were added to the mixture, the liquids were separated, and the organic layer was concentrated under reduced pressure. The obtained residue was purified by silica gel column chromatography (chloroform:methanol=19:1) to give 43 mg of (3S,4S)-4-{N-1-(R)-(3,5-bistrifluoromethylphenyl)ethyl-Nmethyl}aminocarbonyl-3-(4-fluoro-2-methylphenyl)-1-(2-pyrazinyl)piperidine shown in Table 38 below.

### Example 265

In 2 ml of 1,4-dioxane was dissolved 100 mg of (3S,4S)-4-{N-1-(R)-(3,5-bistrifluoromethylphenyl)ethyl-N-methyl}aminocarbonyl-2-(4-fluoro-2-methylphenyl)piperidine, then, 26 mg of 2-chloropyrimidine and 39 μl of diisopropylethylamine were added to the solution and the resulting mixture was stirred at 90° C. overnight. The reaction mixture was cooled to room temperature and concentrated under reduced pressure, and the obtained residue was purified by silica gel column chromatography (n-hexane:ethyl acetate=9:1→2:1) to give 90 mg of (3S,4S)-4-{N-1-(R)-(3,5-bistrifluoromethylphenyl)ethyl-N-methyl}aminocarbonyl-2-(4-fluoro-2-methylphenyl)-1-(2-pyrimidyl)piperidine shown in Table 38 below.

### Example 266

The corresponding starting materials were used and treated in the same manner as in Example 35, to give compounds as shown in Table 38 below.

### Example 267

To 4 ml of a tetrahydrofuran solution containing 100 mg of (3S,4S)-4-{N-1-(R)-(3,5-bistrifluoromethylphenyl)ethyl-N-methyl}-aminocarbonyl-2-(4-fluoro-2-methylphenyl)piperidine were added 0.07 ml of triethylamine and 0.05 ml of methyl chlorocarbonate, and the resulting mixture was stirred at 0° C. for 2 hours. Ethyl acetate and water were added to the mixture, the liquids were separated, and the organic layer was dried over anhydrous magnesium sulfate and concentrated under reduced pressure. The obtained residue was purified by silica gel column chromatography (chloroform:methanol=19:1) to give 60 mg of (3S,4S)-4-{N-1-(R)-(3,5-bistrifluoromethylphenyl)ethyl-N-methyl}aminocarbonyl-3-(4-fluoro-2-methylphenyl)-1-methoxycarbonylpiperidine shown in Table 38 below.

### Example 268

The corresponding starting materials were used and treated in the same manner as in Example 267, to give compounds as shown in Table 38 below.

### Example 269

To 1 ml of a dichloromethane solution containing 50 mg of (3S,4S)-4-{N-1-(S)-(3,5-bistrifluoromethylphenyl)ethyl-N-methyl}-aminocarbonyl-2-(4-fluoro-2-methylphenyl)piperidine were added 12 mg of triphosgene and 0.014 ml of triethylamine at 0° C., and the resulting mixture was stirred at room temperature for 20 minutes and concentrated under reduced pressure. To the obtained residue were added 2 ml of ethylene glycol, 0.014 ml of triethylamine and 0.5 mg of 4-N,N-dimethylaminopyridine, and the resulting mixture was stirred at 40° C. for 16 hours and concentrated under reduced pressure. The obtained residue was purified by silica gel column chromatography to give 52 mg of (3S,4S)-4-{N—(S)-2-(3,5-bistrifluoromethylphenyl)ethyl-N-methyl}aminocarbonyl-3-(4-fluoro-2-methylphenyl)-1-(2-hydroxyethyloxycarbonyl)piperidine shown in Table 39 below.

### Example 270

The corresponding starting materials were used and treated in the same manner as in Example 269, to give compounds as shown in Table 39 below.

### Example 271

To 2.5 ml of a tetrahydrofuran solution containing 51 mg of 4-hydroxytetrahydropyran was added 81 mg of N,N-carbonyldiimidazole, and the resulting mixture was stirred at 70° C. for 2 hours. Ethyl acetate and water were added to the mixture, the liquids were separated, and the obtained organic layer was concentrated under reduced pressure. To the obtained residue were added 2.5 ml of tetrahydrofuran; 60 mg of (3S,4S)-4-{N-1-(R)-(3,5-bistrifluoromethylphenyl)ethyl-N-methyl}aminocarbonyl-2-(4-fluoro-2-methylphenyl)piperidine and 0.028 ml of triethylamine, and the resulting mixture was stirred at 70° C. for 16 hours. Ethyl acetate and water were added to the mixture, the liquids were separated, and the organic layer was concentrated under reduced pressure. The residue was purified by silica gel column chromatography (chloroform:methanol=19:1) to give 55 mg of (3S,4S)-4-{N-1-(R)-(3,5-bistrifluoromethylphenyl)ethyl-N-methyl}aminocarbonyl-3-(4-fluoro-2-methylphenyl)-1-(4-tetrahydropyranyloxycarbonyl)piperidine shown in Table 39 below.

### Example 272

(1) In 2 ml of dichloromethane was dissolved 100 mg of (3S,4S)-4-{N-1-(R)-(3,5-bistrifluoromethylphenyl)ethyl-N-methyl}aminocarbonyl-2-(4-fluoro-2-methylphenyl)piperidine, 49 μl of tert-butyl nitrite was added to the solution, and the mixture was stirred under reflux overnight. 49 μl of tert-butyl nitrite was additionally added to the mixture, the resulting mixture was further refluxed overnight, then, 49 μl of tert-butyl nitrite was additionally added to the same, and the resulting mixture was allowed to stand at room temperature for 4 hours. The reaction mixture was concentrated under reduced pressure, the obtained residue was dissolved in 1 ml of acetic acid and 1 ml of methanol, 107 mg of zinc powder was added to the mixture under ice-cooling, and the resulting mixture was stirred at room temperature for 3 hours. Insoluble materials were filtered off, washed with methanol, and the filtrate and the washing solution were combined and concentrated under reduced pressure. To the residue were added an aqueous saturated sodium hydrogen carbonate solution and dichloromethane, the liquids were separated and the organic layer was concentrated under reduced pressure to give unpurified (3S,4S)-1-amino-4-{N-1-(R)-(3,5-bistrifluoromethylphenyl)ethyl-N-methyl}-aminocarbonyl-2-(4-fluoro-2-methylphenyl)piperidine.
(2) The compound obtained from 50 mg of (3S,4S)-4-{N-1-(R)-(3,5-bistrifluoromethylphenyl)ethyl-N-methyl}aminocarbonyl-2-(4-fluoro-2-methylphenyl)piperidine in the above-mentioned (1) was dissolved in 1 ml of tetrahydrofuran, 11 mg of succinic anhydride was added to the mixture and the resulting mixture was refluxed for 1 hour. The reaction mixture was cooled to room temperature, 12 mg of 1,1′-carbonyldiimidazole was added to the mixture, and the resulting mixture was refluxed for 2 hours. The reaction mixture was cooled to room temperature, and purified by silica gel thin-layer chromatography (developed by and eluted with dichloromethane:ethanol:aqueous ammonia=200:10:1), and lyophilized from tert-butanol to give 36 mg of (3S,4S)-4-{N-1-(R)-(3,5-bistrifluoromethylphenyl)ethyl-N-methyl}aminocarbonyl-1-(2,5-dioxo-pyrrolidin-1-yl)-2-(4-fluoro-2-methylphenyl)piperidine shown in Table 39 below.

### Example 273

The corresponding starting materials were used and treated in the same manner as in Example 272, to give compounds as shown in Table 39 below.

### Example 274

(1) The compound obtained from 50 mg of (3S,4S)-4-{N-1-(R)-(3,5-bistrifluoromethylphenyl)ethyl-N-methyl}aminocarbonyl-2-(4-fluoro-2-methylphenyl)piperidine in Example 272(1) was dissolved in 2 ml of tetrahydrofuran, then, 17 μl of triethylamine and 12 μl of 4-chlorobutyric acid chloride were added to the solution at room temperature, and the mixture was stirred at the same temperature overnight. The reaction mixture was purified by silica gel thin-layer chromatography (developed by chloroform:methanol=9:1, eluted with dichloromethane:ethanol:aqueous ammonia=100:10:1) to give 49 mg of (3S,4S)-4-{N-1-(R)-(3,5-bistrifluoromethylphenyl)ethyl-Nmethyl}aminocarbonyl-1-(4-chlorobutyrylamino)-2-(4-fluoro-2-methylphenyl)piperidine.
(2) In 2 ml of N,N-dimethylformamide was dissolved 49 mg of the compound obtained in the above-mentioned (1), and 4 mg of 70% sodium hydride was added to the solution under ice-cooling and stirring. The mixture was stirred while elevating the temperature to room temperature for 6 hours. To the reaction mixture was added an aqueous saturated ammonium chloride solution, and the liquids were separated by adding dichloromethane. The organic layer was washed with water, and concentrated under reduced pressure. The obtained residue was purified by silica gel thin-layer chromatography (developed by chloroform:methanol=9:1, eluted with dichloromethane:ethanol:aqueous ammonia=100:10:1), and then lyophilized by using tert-butanol to give 35 mg of (3S,4S)-4-{N-1-(R)-(3,5-bistrifluoromethylphenyl)ethyl-N-methyl}aminocarbonyl-2-(4-fluoro-2-methylphenyl)-1-(2-oxo-pyrrolidin-1-yl)piperidine shown in Table 39 below.

### Examples 275 to 277

The corresponding starting materials were used and treated in the same manner as in Example 274, to give compounds as shown in Table 39 below.

### Example 278

The compound obtained from 50 mg of (3S,4S)-4-{N-1-(R)-(3,5-bistrifluoromethylphenyl)ethyl-N-methyl}aminocarbonyl-2-(4-fluoro-2-methylphenyl)piperidine in Example 272(1) was dissolved in 2 ml of tetrahydrofuran, then, 17 μl of triethylamine and 12 μl of divinylsulfone were added to the solution at room temperature, and the mixture was stirred at the same temperature for 5 hours. To the mixture were added 12 μl of divinylsulfone, 12 μl of triethylamine and methanol, and the resulting mixture was stirred at room temperature overnight. 20 μl of divinylsulfone was additionally added to the mixture, and the resulting mixture was stirred at room temperature overnight. The reaction mixture was purified by silica gel thin-layer chromatography (developed by chloroform:methanol=9:1, eluted with dichloromethane:ethanol:aqueous ammonia=100:10:1) to give 20 mg of (3S,4S)-4-{N-1-(R)-(3,5-bistrifluoromethylphenyl)ethyl-N-methyl}aminocarbonyl-1-(1,1-dioxo-thiomorpholine-4-yl)-2-(4-fluoro-2-methylphenyl)piperidine shown in Table 40 below.

### Example 279

The corresponding starting materials were used and treated in the same manner as in Example 278, to give compounds as shown in Table 40 below.

### Examples 280 to 281

The corresponding starting materials were used and treated in the same manner as in Example 272, to give compounds as shown in Table 40 below.

### Example 282

To 10 ml of acetonitrile and 20 ml of water solution containing 50 mg of (3S,4S)-1-amino-4-{N-1-(S)-(3,5-bistrifluoromethylphenyl)ethyl-N-methyl}aminocarbonyl-3-(4-fluoro-2-methylphenyl)piperidine was added a (2-oxoethoxy)acetaldehyde, which was prepared by adding 86 mg of sodium periodate to 1 ml of water containing 42 mg of 1,4-anhydroeryerythritol followed by stirring at room temperature overnight, and 63 mg of sodium cyanoborohydride, the resulting mixture was stirred at room temperature for 2 hours. The reaction mixture was concentrated under reduced pressure, and to the obtained residue were added ethyl acetate and an aqueous saturated sodium hydrogen carbonate solution, the liquids were separated. The organic layer was washed with brine, dried over anhydrous magnesium sulfate and concentrated under reduced pressure. The obtained residue was purified by thin-layer silica gel column chromatography (developed by chloroform:methanol=9:1, eluted with dichloromethane:ethanol:aqueous ammonia=200:10:1), and lyophilized by using tert-buthanol to give 18.3 mg of (3S,4S)-4-{N-1-(S)-(3,5-bistrifluoromethylphenyl)ethyl-N-methyl}aminocarbonyl-3-(4-fluoro-2-methylphenyl)-1-morpholinopiperidine shown in Table 40 below.

### Example 283

The corresponding starting materials were used and treated in the same manner as in Example 282, to give compounds as shown in Table 40 below.

### Example 284

(1) In 1 ml of dimethylsulfoxide were dissolved 50 mg of (3S,4S)-4-{N-1-(R)-(3,5-bistrifluoromethylphenyl)ethyl-N-methyl}aminocarbonyl-3-(4-fluoro-2-methylphenyl)piperidine and 21 mg of methyl 2-chloronicotinate, 14 mg of potassium carbonate was added to the mixture, and the resulting mixture was stirred at 100° C. overnight. After cooling the reaction mixture to room temperature, ethyl acetate and an aqueous saturated sodium hydrogen carbonate solution were added to the reaction mixture, the liquids were separated. The organic layer was washed with brine, dried over anhydrous magnesium sulfate and concentrated under reduced pressure. The obtained residue was purified by thin-layer silica gel column chromatography (developed by chloroform:methanol=9:1, eluted with dichloromethane:ethanol:aqueous ammonia=100:10:1) to give 53 mg of (3S,4S)-4-{N-1-(R)-(3,5-bistrifluoromethylphenyl)ethyl-N-methyl}-aminocarbonyl-3-(4-fluoro-2-methylphenyl)-1-(5-methoxycarbonylpyridin-2-yl)piperidine.
(2) In 1 ml of a ethanol was dissolved 53 mg of the compound obtained in the above-mentioned (1), 127 μl of a 1M aqueous sodium hydroxide solution was added to the mixture, and the resulting mixture was stirred at room temperature for 3 days. The reaction mixture was neutralized by 127 μl of a 1M aqueous hydrochloric acid solution and concentrated under reduced pressure. To 2 ml of a tetrahydrofurane were added the obtained residue, 68 mg of 1-hydroxybenzotriazole and 97 mg of 1-(3-dimethylaminopropyl)-3-ethylcarbodiimide hydrochloride, and the resulting mixture was stirred at room temperature overnight. To the reaction mixture was added 45 mg of a 50% aqueous dimethylamine solution and stirred at room temperature overnight. The resulting mixture was directly purified by thin-layer silica gel column chromatography (developed by chloroform:methanol=19:1, eluted with dichloromethane:ethanol:aqueous ammonia=100:10:1) to give 38 mg of (3S,4S)-4-{N-1-(R)-(3,5-bistrifluoromethylphenyl)ethyl-N-methyl}aminocarbonyl-1-(5-N,N-dimethylaminocarbonylpyridin-2-yl)-3-(4-fluoro-2-methylphenyl)piperidine as shown in Table 40 below.

### Examples 285 to 295

The corresponding starting materials were used and treated in the same manner as in Example 284, to give compounds as shown in Table 40 to 41 below.

### Examples 296 to 298

The corresponding starting materials were used and treated in the same manner as in Example 1, to give compounds as shown in Tables 41 to 42 below.

### Example 299

The corresponding starting materials were used and treated in the same manner as in Example 27, to give compounds as shown in Table 42 below.

### Examples 300 to 301

The corresponding starting materials were used and treated in the same manner as in Example 1 and the obtained two kinds of diastereomer compounds were separated by silica gel column chromatography, to give compounds as shown in Table 43 below.

### Examples 302 to 305

The corresponding starting materials were used and treated in the same manner as in Example 119, to give compounds as shown in Table 44 below.

### Examples 306 to 309

The corresponding starting materials were used and treated in the same manner as in Example 6, to give compounds as shown in Table 44 below.

### Examples 310 to 313

The corresponding starting materials were used and treated in the same manner as in Example 13, to give compounds as shown in Tables 44 to 45 below.

### Examples 314 to 320

The corresponding starting materials were used and treated in the same manner as in Example 27, to give compounds as shown in Table 45 below.

### Examples 321 to 323

The corresponding starting materials were used and treated in the same manner as in Example 228, to give compounds as shown in Table 46 below.

### Examples 324 to 327

The corresponding starting materials were used and treated in the same manner as in Example 1, to give compounds as shown in Table 47 below.

### Examples 328 to 331

The corresponding starting materials were used and treated in the same manner as in Example 119, to give compounds as shown in Table 48 below.

### Examples 332 to 335

The corresponding starting materials were used and treated in the same manner as in Example 6, to give compounds as shown in Table 48 below.

### Examples 336 to 338

The corresponding starting materials were used and treated in the same manner as in Example 13, to give compounds as shown in Table 49 below.

### Examples 339 to 341

The corresponding starting materials were used and treated in the same manner as in Example 27, to give compounds as shown in Table 49 below.

### Example 342

The corresponding starting materials were used and treated in the same manner as in Example 1, to give compounds as shown in Table 50 below.

### Example 343

The corresponding starting materials were used and treated in the same manner as in Example 1 and the obtained two kinds of diastereomer compounds were separated by silica gel column chromatography, to give compounds as shown in Table 50 below.

### Examples 344 to 345

The corresponding starting materials were used and treated in the same manner as in Example 119, to give compounds as shown in Table 50 below.

### Examples 346 to 349

The corresponding starting materials were used and treated in the same manner as in Example 6, to give compounds as shown in Table 50 below.

### Examples 350 to 354

The corresponding starting materials were used and treated in the same manner as in Example 13, to give compounds as shown in Table 51 below.

### Examples 355 to 358

The corresponding starting materials were used and treated in the same manner as in Example 27, to give compounds as shown in Tables 51 to 52 below.

### Example 359

The corresponding starting materials were used and treated in the same manner as in Example 212, to give compounds as shown in Table 52 below.

### Reference Example 1

(1) 320 ml of a tetrahydrofuran solution containing 22.4 ml of diisopropyl amine was cooled to −70° C. or lower in a dry ice-acetone bath, then, 100 ml of n-butyl lithium (1.6M hexane solution) was added dropwise to the solution, and the resulting mixture was stirred at the same temperature for 30 minutes. To the solution was added dropwise 250 ml of a tetrahydrofuran solution containing 25 g of 3-bromopyridine over 4 hours. After completion of the dropwise addition, the mixture was stirred at −70° C. or lower for further 1 hour. To the solution was added 8.8 g of dry ice the surface of which had been well polished and finely pulverized, and the mixture was stirred for 1 hour, and the temperature of the mixture was gradually elevated to room temperature. After the solvent and excess carbon dioxide were completely removed under reduced pressure, the residue was dissolved in 300 ml of N,N-dimethylformamide, 27.6 g of potassium carbonate and 12.6 ml of methyl iodide were added to the solution, and the mixture was stirred at room temperature for 16 hours. Ethyl acetate and an aqueous sodium bicarbonate solution were added to the mixture, liquids were separated, and the organic layer was washed with water and brine. The organic layer was dried over anhydrous magnesium sulfate and concentrated under reduced pressure. The obtained residue was purified by silica gel column chromatography (n-hexane:ethyl acetate=4:1) to give 13.5 g of methyl 3-bromoisonicotinate shown in Table 53 below.
(2) To 120 ml of N,N-dimethylformamide solution containing 12 g of the compound obtained in the above-mentioned (1) were added 9.3 g of 4-fluoro-2-methylphenylboric acid, 19.6 g of cesium carbonate, 1.12 g of palladium acetate and 2.63 g of triphenylphosphine, and the resulting mixture was stirred at 70° C. for 1 hour. After completion of the reaction, ethyl acetate and brine were added to the reaction mixture, and insoluble materials were filtered off. The filtrate was washed with brine and water, dried over anhydrous magnesium sulfate, and concentrated under reduced pressure. The obtained residue was purified by silica gel column chromatography (n-hexane:ethyl acetate=4:1) to give 7.9 g of methyl 3-(4-fluoro-2-methylphenyl)isonicotinate shown in Table 53 below.
(3) To 100 ml of a methanol solution containing 2.5 g the compound obtained in the above-mentioned (2) were added 600 mg of platinum oxide and 8 ml of conc. hydrochloric acid. Then, the mixture was stirred under hydrogen atmosphere at 101 kPa at room temperature for 24 hours. To the solution was added 100 ml of water, the mixture was filtered through Celite, and the filtrate was concentrated under reduced pressure. The remained aqueous solution was neutralized by sodium carbonate, aqueous ammonia was further added, and the mixture was extracted twice with chloroform. The combined organic layers were dried over anhydrous sodium sulfate and concentrated under reduced pressure. To 25 ml of a dichloromethane solution of the residue was added 5 g of di-tert-butyldicarbonate, and the resulting mixture was stirred at room temperature for 1 hour and concentrated under reduced pressure. The obtained residue was purified by silica gel column chromatography (n-hexane:ethyl acetate=85:15) to give 1.3 g of cis-1-tert-butoxycarbonyl-3-(4-fluoro-2-methylphenyl)-4-methoxycarbonylpiperidine shown in Table 53 below.
(4) In 5 ml of methanol and 5 ml of tetrahydrofuran was dissolved 1.3 g of the compound obtained in the above-mentioned (3), 5 ml of a 2M aqueous sodium hydroxide solution was added to the solution and the mixture was stirred at room temperature for 16 hours. The mixture was neutralized by a 2M aqueous hydrochloric acid solution, and extracted twice with chloroform. The organic layer was dried over anhydrous sodium sulfate, and concentrated under reduced pressure. The obtained residue was dried under reduced pressure to give 560 mg of a mixture of cis-1-tert-butoxycarbonyl-4-carboxyl-3-(4-fluoro-2-methylphenyl)piperidine, and trans-1-tert-butoxycarbonyl-4-carboxyl-3-(4-fluoro-2-methylphenyl)piperidine (cis isomer:trans isomer=56:44) shown in Table 53 below.
(5) In 100 ml of methanol was dissolved 10.5 g of cis-1-tert-butoxycarbonyl-3-(4-fluoro-2-methylphenyl)-4-methoxycarbonylpiperidine, 11.4 ml of a methanol solution containing 28% sodium methoxide was added to the solution and the resulting mixture was stirred under reflux for 3 hours. To the reaction mixture were added 50 ml of tetrahydrofuran and a 2M aqueous sodium hydroxide solution, and the resulting mixture was stirred overnight. After the mixture was neutralized by a 2M aqueous hydrochloric acid solution, chloroform was added to the mixture, and the organic layer was washed with water and saturated brine. The organic layer was dried over anhydrous sodium sulfate and concentrated under reduced pressure. The obtained residue was crystallized from ethyl acetate and n-hexane to give 7.15 g of trans-1-tert-butoxycarbonyl-4-carboxyl-3-(4-fluoro-2-methylphenyl)piperidine shown in Table 54 below.
(6) In 2500 ml of ethyl acetate was dissolved 84.3 g of trans-1-tert-butoxycarbonyl-4-carboxyl-3-(4-fluoro-2-methylphenyl)piperidine, 1500 ml of an ethyl acetate solution containing 15.1 g of (R)-phenethylamine was added dropwise to the solution at room temperature over 1.5 hours. The precipitated white salt was collected by filtration, washed twice with ethyl acetate, and washed with a mixed solvent of 200 ml of diisopropyl ether and 400 ml of methanol. A saturated aqueous citric acid solution was added to the white salt, the mixture was extracted with chloroform, washed with saturated brine, dried over anhydrous magnesium sulfate and concentrated under reduced pressure to give 26.3 g of (a) (3R,4R)-1-tert-butoxycarbonyl-4-carboxyl-3-(4-fluoro-2-methylphenyl)piperidine shown in Table 54 below. The filtrates obtained by the above-mentioned operations were combined, a saturated aqueous citric acid solution was added thereto, and the mixture was extracted with chloroform. The organic layer was washed with brine, dried over anhydrous magnesium sulfate, and concentrated under reduced pressure. The obtained residue was dissolved in 2500 ml of ethyl acetate, 1500 ml of an ethyl acetate solution containing 15.1 g of (S)-phenethylamine was added dropwise to the solution at room temperature over 1.5 hours. Precipitated white salt was collected by filtration, washed twice with ethyl acetate, a saturated aqueous citric acid solution was added to the white salt, and the mixture was extracted with chloroform. The organic layer was washed with brine, dried over anhydrous magnesium sulfate, and concentrated under reduced pressure. The obtained residue was crystallized from ethyl acetate and n-hexane to give 28.1 g of (b) (3S,4S)-1-tert-butoxycarbonyl-4-carboxyl-3-(4-fluoro-2-methylphenyl)piperidine shown in Table 54 below.

### Reference Example 2

(1) To a slurry comprising 500 ml of a 1,2-dichloroethane solution containing 50 g of isonicotinic acid chloride hydrochloride and cooled to 0° C. were gradually added dropwise 31.4 g of aniline and 50 ml of a 1,2-dichloroethane solution containing 60 g of triethylamine over 25 minutes or longer. After stirring the mixture at room temperature for 30 minutes, it was stirred under reflux for 1.5 hours. To the reaction mixture was added 100 ml of water and the mixture was gradually cooled to 0° C. The formed precipitates were collected by filtration, dried under reduced pressure, washed with diethyl ether, and dried under reduced pressure to give 45 g of N-phenylisonicotinic amide shown in Table 55 below.
(2) 640 ml of a tetrahydrofuran solution containing 32 g of the compound obtained in the above-mentioned (1) was cooled to −78° C., 13 ml of n-butyl lithium (1.6M hexane solution) was added dropwise to the solution and the resulting mixture was stirred for 0.5 hour. The temperature of the reaction mixture was gradually elevated up to 0° C., and the mixture was stirred for 1.5 hours. The mixture was again cooled to −78° C., 120 ml of a tetrahydrofuran solution containing 40 g of iodine was added dropwise to the mixture, and the resulting mixture was stirred for 3 hours. To the reaction mixture were added ethyl acetate and water, the liquids were separated, and the organic layer was dried over anhydrous magnesium sulfate. The organic layer was concentrated under reduced pressure, and the obtained residue was triturated by a mixed solvent of dichloromethane and diisopropyl ether to give 31 g of N-phenyl-3-iodoisonicotinic amide shown in Table 55 below.
(3) To 25 g of the compound obtained in the above-mentioned (2) was added 200 ml of about 25% aqueous hydrochloric acid solution, and the resulting mixture was stirred under reflux for 16 hours. The reaction mixture was cooled to 0° C., and the formed precipitates were collected by filtration. The collected precipitates were washed with a small amount of water to give 14.5 g of 3-iodoisonicotinic acid hydrochloride shown in Table 55 below.
(4) To 125 ml of an ethyl acetate solution containing 14.5 g of the compound obtained in the above-mentioned (3) were added one drop of N,N-dimethylformamide, and then, 9.3 g of thionyl chloride, and the resulting mixture was stirred under reflux for 1 hour. The reaction mixture was concentrated under reduced pressure, 100 ml of methanol was added to the residue, and the mixture was stirred under reflux for 2 hours. The reaction mixture was concentrated under reduced pressure, the obtained residue was triturated by diethyl ether and dried under reduced pressure to give 13.2 g of methyl 3-iodoisonicotinate shown in Table 55 below.
(5) 13.2 g of the compound obtained in the above-mentioned (4) and corresponding starting materials were used and treated in the same manner as in Reference example 1(2), to give 7.7 g of methyl 3-(4-fluorophenyl)isonicotinate shown in Table 55 below.
(6) 7.0 g of the compound obtained in the above-mentioned (5) and corresponding starting materials were used and treated in the same manner as in Reference example 1(3), to give 6.5 g of cis-1-tert-butoxycarbonyl-3-(4-fluorophenyl)-4-methoxycarbonylpiperidine shown in Table 55 below.
(7) 6 g of the compound obtained in the above-mentioned (6) and corresponding starting materials were used and treated in the same manner as in Reference example 1(4) to give 5.8 g of a mixture of cis-1-tert-butoxycarbonyl-4-carboxyl-3-(4-fluorophenyl)piperidine and trans-1-tert-butoxycarbonyl-4-carboxyl-3-(4-fluorophenyl)piperidine (cis isomer:trans isomer=54:46) shown in Table 56 below.

### Reference Example 3

(1) The compound obtained in Reference example 2(4) and corresponding starting materials were used and treated in the same manner as in Reference example 2(5), to give methyl 3-(2,4-difluorophenyl)isonicotinate shown in Table 56 below.
(2) The compound obtained in the above-mentioned (1) and corresponding starting materials were used and treated in the same manner as in Reference example 2(6), to give cis-1-tert-butoxycarbonyl-3-(2,4-difluorophenyl)-4-methoxycarbonylpiperidine shown in Table 56 below.
(3) The compound obtained in the above-mentioned (2) was used and treated in the same manner as in Reference example 1(4), to give (a) (3S,4S)-1-tert-butoxycarbonyl-4-carboxyl-3-(2,4-difluorophenyl)piperidine and (b) (3R,4R)-1-tert-butoxycarbonyl-4-carboxyl-3-(2,4-difluorophenyl)piperidine shown in Table 56 below.

### Reference Example 4

(1) To 200 ml of a tetrahydrofuran solution containing 12.8 g of 3,5-bistrifluoromethylacetophenone was added dropwise 25 ml of a tetrahydrofuran solution containing 3M of methyl magnesium at −20° C. and the resulting mixture was stirred for 2 hours. To the reaction mixture were added ammonium chloride and ethyl acetate, the mixture was concentrated under reduced pressure, 27 ml of trimethylsilyl cyanide and 16 ml of conc. sulfuric acid were added to the residue at −20° C., and the resulting mixture was stirred for 3 hours. The reaction mixture was dropped in ice, neutralized by a 1M aqueous sodium hydroxide solution and extracted with chloroform, and the organic layer was dried over anhydrous magnesium sulfate and evaporated under reduced pressure. The obtained residue was purified by silica gel column chromatography (n-hexane:ethyl acetate=80:20-50:50) to give 5.47 g of N-{1-(3,5-bistrifluoromethylphenyl)-1-methyl-ethyl}-N-formamide shown in Table 57 below.
(2) To 150 ml of a N,N-dimethylformamide solution containing 5.47 g of the compound obtained in the above-mentioned (1) was added 800 mg of sodium hydride (60% in oil) under nitrogen atmosphere, and the resulting mixture was stirred for 1 hour and 10 minutes. Then, 7.1 g of methyl iodide was added to the mixture, and the resulting mixture was stirred for 3 hours and 45 minutes, and concentrated under reduced pressure. The obtained residue was purified by basic silica gel column chromatography (n-hexane:ethyl acetate=85:15→60:40) to give N-{1-(3,5-bistrifluoromethylphenyl)-1-methyl-ethyl}-N-methylformamide shown in Table 57 below.
(3) To 50 ml of an ethanol solution containing the compound obtained in the above-mentioned (2) was added a 47% aqueous hydrogen bromide solution at room temperature, and after stirring overnight, the resulting mixture was raised to 60° C., and stirred for 2 days. The reaction mixture was dropped into an aqueous sodium hydrogen carbonate solution in which ice was charged, extracted with chloroform, and concentrated under reduced pressure. To the obtained residue was added dichloromethane, insoluble materials were removed by filtration, and the filtrate was concentrated under reduced pressure to give 3.00 g of N-{1-(3,5-bistrifluoromethylphenyl)-1-methyl-ethyl}-N-methylamine shown in Table 57 below.

### Reference Example 5

To 9 ml of an ethyl acetate solution containing 2.02 g of (3S,4S)-1-tert-butoxycarbonyl-4-carboxyl-3-(4-fluoro-2-methylphenyl)piperidine was added 27 ml of an ethyl acetate solution containing 4M of hydrochloric acid, and the resulting mixture was stirred at room temperature for 1.5 hours, and concentrated under reduced pressure. Water was added to the residue, the aqueous layer was basified with an aqueous sodium carbonate solution, and the precipitated solid was collected by filtration. To a solution of the obtained solid dissolved in 25 ml of water and 25 ml of tetrahydrofuran were added 1.9 g of sodium carbonate and 1.12 g of benzylchloroformate, the resulting mixture was stirred at room temperature for 16 hours, and concentrated under reduced pressure. To the residue were added ethyl acetate, water and a saturated aqueous citric acid solution, and the liquids were separated. The obtained organic layer was washed with brine, dried over anhydrous sodium sulfate, and concentrated under reduced pressure. The obtained residue was purified by silica gel column chromatography (chloroform-chloroform:methanol=19:1) to give 1.11 g of (3S,4S)-1-benzyloxycarbonyl-4-carboxyl-3-(4-fluoro-2-methylphenyl)piperidine shown in Table 57 below.

### Reference Example 6

To 30 ml of a dichloromethane solution containing 4.8 g of ethyl isonicopetinate were added 3.5 ml of propionic chloride and 5.6 ml of triethylamine at 0° C., and the mixture was stirred for 2 hours. To the reaction mixture were added an aqueous sodium bicarbonate solution and chloroform, and the liquids were separated. The organic layer was successively washed with an aqueous hydrochloric acid solution and then brine, dried over anhydrous magnesium sulfate, and concentrated under reduced pressure. To the obtained residue were added 30 ml of methanol and 30 ml of a 2M aqueous sodium hydroxide solution, and the mixture was stirred at room temperature for 16 hours. The aqueous layer obtained by removing methanol from the reaction mixture under reduced pressure was washed with ether, and the aqueous layer was slightly acidified by hydrochloric acid and citric acid. The aqueous layer was extracted twice with chloroform, and the organic layer was dried over anhydrous magnesium sulfate and concentrated under reduced pressure. The obtained residue was crystallized from diisopropyl ether to give 2.5 g of 1-propionylpiperidine-4-carboxylic acid shown in Table 57 below.

### Reference Example 7

The corresponding starting materials were used and treated in the same manner as in Reference example 6, to give 1-isobutyroylpiperidine-4-carboxylic acid as shown in Table 57 below.

### Reference Example 8

To 1.5 ml of a 1-propanol solution containing 500 mg of 2-bromo-2-methylpropionic acid were added 600 mg of morpholine and 0.55 ml of triethylamine, and the mixture was stirred under reflux for 16 hours. After the mixture was cooled to room temperature, 0.3 ml of a 10M aqueous sodium hydroxide solution was added to the mixture and the resulting mixture was concentrated under reduced pressure. The obtained residue was subjected to azeotropic distillation with toluene, and then vacuum dried. To the obtained residue was added 1-propanol to carry out trituration to give 380 mg of 2-methyl-2-(4-morpholinyl)propionic acid sodium salt shown in Table 57 below.

### Reference Example 9

(1) To 100 ml of a toluene solution containing 60 ml of a hexane solution with 2M trimethyl aluminum was added dropwise 40 ml of a toluene solution containing 10.2 g of 4-ethoxycarbonylcyclohexanone at 0° C., and the resulting mixture was stirred for 30 minutes. To the reaction mixture were added water and an aqueous saturated sodium hydrogen carbonate solution and the liquids were separated. The organic layer was washed twice with water, and once with saturated brine, dried over anhydrous magnesium sulfate, and concentrated under reduced pressure. The obtained residue was purified by silica gel column chromatography (hexane:ethyl acetate=85:15-75:25) to give 3.43 g of trans-4-ethoxycarbonyl-1-methylcyclohexanol shown in Table 58 below.
(2) To 24 ml of an ethanol solution containing 2.24 g of the compound obtained in the above-mentioned (1) were added 580 mg of sodium hydroxide and 12 ml of water, and the mixture was stirred at room temperature overnight. The reaction mixture was concentrated under reduced pressure, the residue was acidified with a 2M aqueous hydrochloric acid solution, and extracted three times with chloroform. The organic layer was dried over ahydrous magnesium, sulfate, and concentrated under reduced pressure to give 1.65 g of trans-4-carboxyl-1-methylcyclohexanol shown in Table 58 below.

### Reference Example 10

To 5 ml of a N,N-dimethylformaldehyde solution containing 800 mg of methyl cyclohexane-1,4-dicarboxylate were added 3.84 g of 1-[3-(dimethylamino)propyl]-3-ethylcarbodiimide hydrochloride, 3.06 g of 1-hydroxybenzotriazole and 1.5 ml of 50% aqueous dimethylamine solution, and the mixture was stirred at room temperature for 16 hours. To the reaction mixture were added ethyl acetate and brine, and the liquids were separated. The organic layer was washed twice with an aqueous sodium bicarbonate solution, dried over anhydrous magnesium sulfate, and concentrated under reduced pressure. To 680 mg of the residue were added 10 ml of methanol and 10 ml of an aqueous 2M sodium hydroxide solution, and the mixture was stirred at room temperature for 16 hours. The mixture was neutralized by 2M aqueous hydrochloric acid solution, and methanol was removed under reduced pressure. The aqueous layer was extracted twice with chloroform, and the combined organic layer was dried over anhydrous magnesium sulfate and concentrated under reduced pressure. The obtained residue was dried under vacuo to give 430 mg of 4-dimethylcarbamoylcyclohexanecarboxylic acid shown in Table 58 below.

### Reference Example 11

To 20 ml of a dichloromethane solution containing 2 g of 2-chloroethanesulfonyl chloride were added 4 ml of pyrrolidine and 4 ml of triethylamine, and the mixture was stirred at 0° C. for 1 hour. To the reaction mixture were added chloroform and 1M aqueous hydrochloric acid solution, and the liquids were separated. The organic layer was washed with water, dried over anhydrous magnesium sulfate, and concentrated under reduced pressure. The obtained residue was purified by silica gel column chromatography (hexane:ethyl acetate=85:15) to give 580 mg of 1-ethenesulfonylpyrrolidine shown in Table 58 below.

### Reference Example 12

(1) To 10 ml of a tetrahydrofuran solution containing 800 mg of magnesium powder and 20 mg of iodine was added dropwise 90 ml of a tetrahydrofuran-solution containing 9 g of 3,5-bistrifluoromethylbromobenzene under reflux, the resulting mixture was stirred for 2 hours. After cooling the reaction mixture to −78° C., 10 ml of a tetrahydrofuran solution containing 3 g propionyl aldehyde was added dropwise to the reaction mixture, and the resulting mixture was stirred for 2 hours. After elevating the reaction mixture to room temperature, to the mixture were added an aqueous ammonium chloride solution and ethyl acetate, and the liquids were separated. The organic layer was washed with brine, dried over anhydrous magnesium sulfate, and concentrated under reduced pressure. The obtained residue was purified by silica gel column chromatography (n-hexane:ethyl acetate=80:20) to give 5.3 g of 1-(3,5-bistrifluoromethylphenyl)-1-propernol as shown in Table 58 below.
(2) To 100 ml of a dichloromethane solution containing 5.3 g of the compound obtained in the above-mentioned (1) and 3 ml of triethylamine was added 1.6 ml of methanesulfonyl chloride at 0° C., and the resulting mixture was stirred for 3 hours. To the reaction mixture were added water and chloroform, and the liquids were separated. The aqueous layer was extracted again with chloroform. The combined organic layers were dried over anhydrous magnesium sulfate, and concentrated under reduced pressure. The obtained residue was purified by silica gel column chromatography (n-hexane:ethyl acetate=80:20) to give 4.3 g of methane sulfonic acid 1-(3,5-bistrifluoromethylphenyl)-1-propyl ester as shown in Table 58 below.
(3) To 100 ml of an acetonitrile solution containing 3.5 g of the compound obtained in the above-mentioned (2) was added 1.3 g of sodium azide, the resulting mixture was stirred under reflux for 3 hours. After cooling the reaction mixture to room temperature, the mixture was concentrated under reduced pressure. To the obtained residue were added water and ethyl acetate, and the liquids were separated. The organic layer was dried over anhydrous magnesium sulfate, and concentrated under reduced pressure. The obtained residue was purified by silica gel column chromatography (n-hexane:ethyl acetate=20:1) to give 2.3 g of 1-(1-azidepropyl)-3,5-bistrifluoromethylbenzene as shown in Table 58 below.
(4) To 40 ml of a methanol solution containing 2.2 g of the compound obtained in the above-mentioned (3) was added 200 mg of 10% palladium carbon, the mixture was stirred under hydrogen atmosphere at room temperature for 8 hours. The reaction mixture was filtered, and filtrate was concentrated under reduced pressure to give 1-(3,5-bistrifluoromethylphenyl)propylamine as shown in Table 58 below.

### Reference Example 13

The corresponding starting materials were used and treated in the same manner as in Reference example 12, to give compounds as shown in Table 59 below.

TABLE 1

| Example | | |
|---|---|---|
| No. | Structural formula | MS |
| 1(a) | 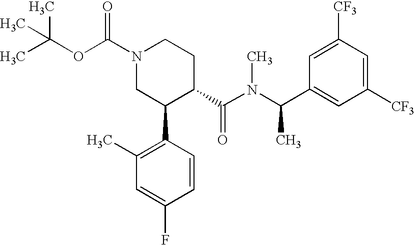 | 591 (M⁺ + 1) |
| 1(b) | 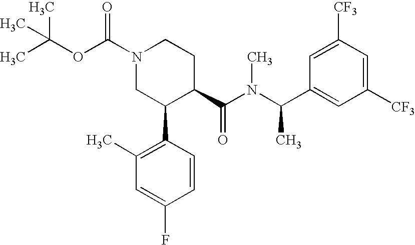 | 591 (M⁺ + 1) |
| 2 | 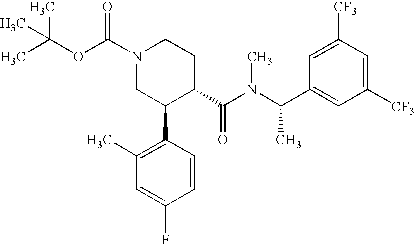 | 591 (M⁺ + 1) |

TABLE 2

| Example | | |
|---|---|---|
| No. | Structural formula | MS |
| 3(a) | 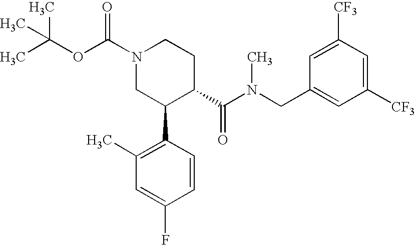 and 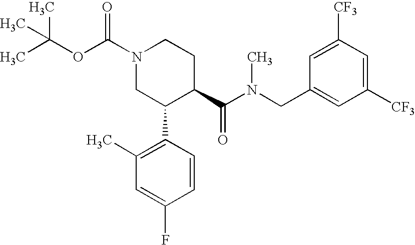 | 577 (M⁺ + 1) |
| 3(b) | 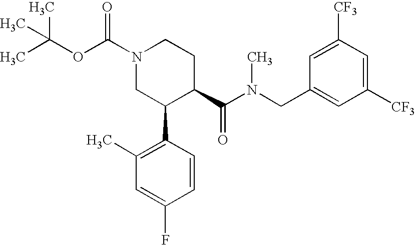 and 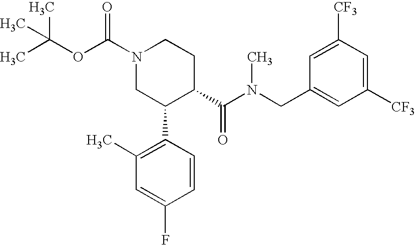 | 577 (M⁺ + 1) |

TABLE 3

| Example | | |
|---|---|---|
| No. | Structural formula | MS |
| 4(a) | 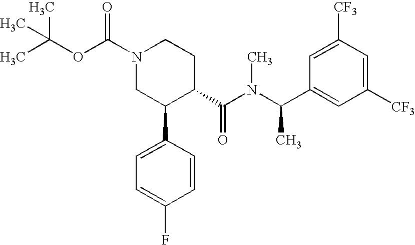 | 577 (M⁺ + 1) |
| 4(b) | 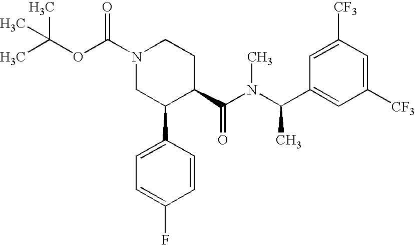 | 577 (M⁺ + 1) |
| 5 | 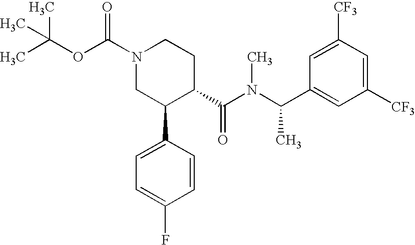 | 577 (M⁺ + 1) |

TABLE 4

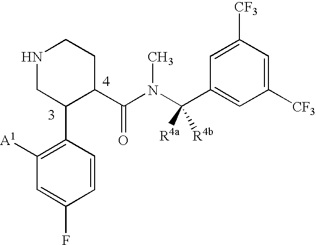

| Example | | | | | |
|---|---|---|---|---|---|
| No. | Configuration | A¹ | R^{4a} | R^{4b} | MS |
| 6 | (3S, 4S) | CH₃ | H | H | 477 (M⁺ + 1) |
| | and | | | | |
| | (3R, 4R) | | | | |
| 7 | (3S, 4S) | CH₃ | CH₃ | H | 491 (M⁺ + 1) |
| 8 | (3S, 4S) | CH₃ | H | CH₃ | 491 (M⁺ + 1) |
| 9 | (3S, 4R) | CH₃ | H | H | 477 (M⁺ + 1) |
| | and | | | | |
| | (3R, 4S) | | | | |
| 10 | (3S, 4S) | H | CH₃ | H | 477 (M⁺ + 1) |
| 11 | (3S, 4R) | H | CH₃ | H | 477 (M⁺ + 1) |
| 12 | (3S, 4S) | H | H | CH₃ | 477 (M⁺ + 1) |

TABLE 5

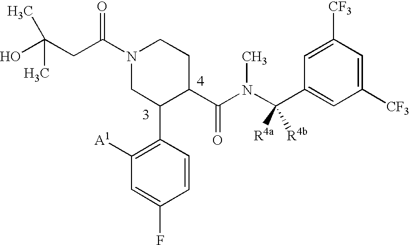

| Example | | | | | |
|---|---|---|---|---|---|
| No. | Configuration | A¹ | R^{4a} | R^{4b} | MS |
| 13 | (3S, 4S) | CH₃ | H | H | 577 (M⁺ + 1) |
| | and | | | | |
| | (3R, 4R) | | | | |
| 14 | (3S, 4S) | CH₃ | CH₃ | H | 591 (M⁺ + 1) |
| 15 | (3S, 4S) | CH₃ | H | CH₃ | 591 (M⁺ + 1) |
| 16 | (3S, 4S) | H | CH₃ | H | 577 (M⁺ + 1) |
| 17 | (3S, 4S) | H | H | CH₃ | 577 (M⁺ + 1) |

TABLE 6

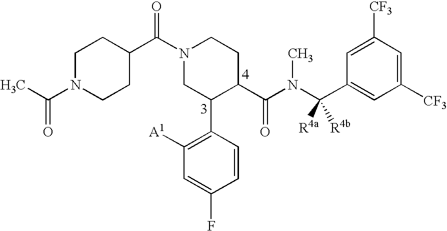

| Example | | | | | |
|---|---|---|---|---|---|
| No. | Configuration | A¹ | R^{4a} | R^{4b} | MS |
| 18 | (3S, 4S) | CH₃ | H | H | 630 (M⁺ + 1) |
| | and | | | | |
| | (3R, 4R) | | | | |
| 19 | (3S, 4R) | CH₃ | H | H | 630 (M⁺ + 1) |
| | and | | | | |
| | (3R, 4S) | | | | |
| 20 | (3S, 4S) | CH₃ | CH₃ | H | 644 (M⁺ + 1) |
| 21 | (3S, 4S) | CH₃ | H | CH₃ | 644 (M⁺ + 1) |
| 22 | (3S, 4S) | H | CH₃ | H | 630 (M⁺ + 1) |
| 23 | (3S, 4R) | H | H | CH₃ | 630 (M⁺ + 1) |
| 24 | (3S, 4S) | H | CH₃ | H | 630 (M⁺ + 1) |

TABLE 7

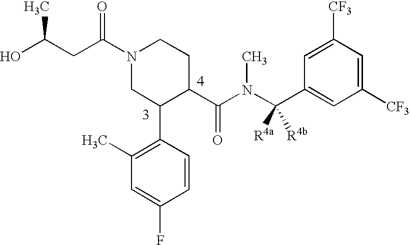

| Example No. | R^{4a} | R^{4b} | MS |
|---|---|---|---|
| 25 | CH₃ | H | 577 (M⁺ + 1) |
| 26 | H | CH₃ | 577 (M⁺ + 1) |

TABLE 8

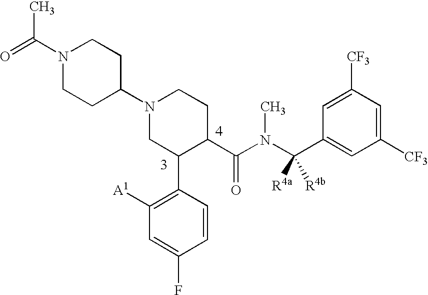

| Example | | | | | |
|---|---|---|---|---|---|
| No. | Configuration | A¹ | R^{4a} | R^{4b} | MS |
| 27 | (3S, 4R) | CH₃ | H | H | 602 (M⁺ + 1) |
| | and | | | | |
| | (3R, 4S) | | | | |
| 28 | (3S, 4S) | CH₃ | CH₃ | H | 616 (M⁺ + 1) |
| 29 | (3S, 4S) | CH₃ | H | CH₃ | 616 (M⁺ + 1) |
| 30 | (3S, 4S) | H | CH₃ | H | 602 (M⁺ + 1) |
| 31 | (3S, 4S) | H | H | CH₃ | 602 (M⁺ + 1) |

TABLE 9

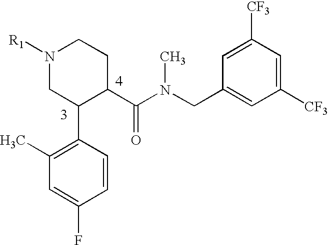

| Example | | | |
|---|---|---|---|
| No. | Configuration | R¹ | MS |
| 32 | (3S, 4S)and(3R, 4R) | 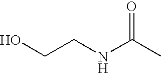 | 564(M⁺ + 1) |
| 33 | (3S, 4S)and(3R, 4R) | 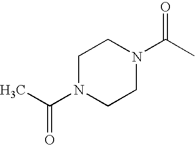 | 631(M⁺ + 1) |
| 34 | (3S, 4R)and(3R, 4S) | 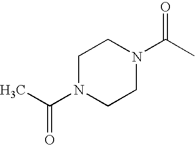 | 631(M⁺ + 1) |
| 35 | (3S, 4S)and(3R, 4R) | —SO₂CH₃ | 555(M⁺ + 1) |

TABLE 10

| Example | | |
|---|---|---|
| No. | Structural formula | MS |
| 36(a) | 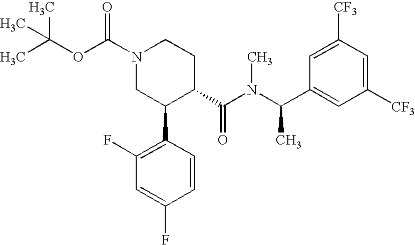 | 595 (M⁺ + 1) |
| 36(b) | 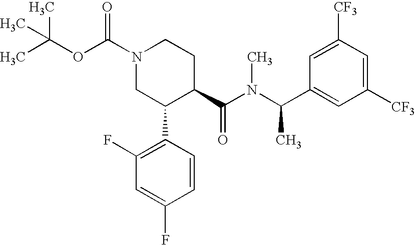 | 595 (M⁺ + 1) |
| 37(1) | 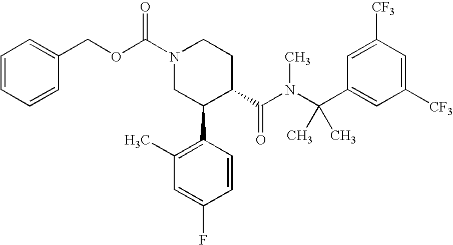 | 639 (M⁺ + 1) |
| 37(2) | 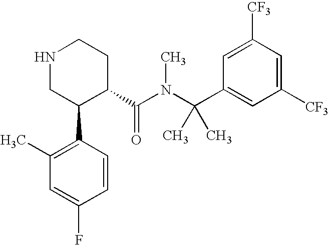 | 505 (M⁺ + 1) |

TABLE 11

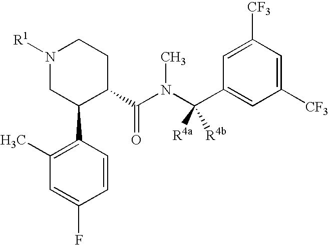

| Example | | | | |
|---|---|---|---|---|
| No. | R¹ | R^{4a} | R^{4b} | MS |
| 38 | 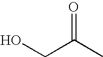 | CH₃ | H | 549 (M⁺ + 1) |
| 39 | 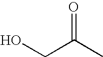 | H | CH₃ | 549 (M⁺ + 1) |
| 40 | 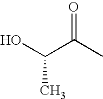 | CH₃ | H | 563 (M⁺ + 1) |
| 41 | 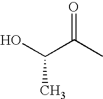 | H | CH₃ | 563 (M⁺ + 1) |
| 42 | 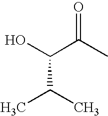 | CH₃ | H | 591 (M⁺ + 1) |
| 43 | 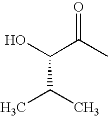 | H | CH₃ | 591 (M⁺ + 1) |
| 44 | 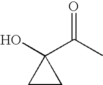 | CH₃ | H | 575 (M⁺ + 1) |
| 45 | 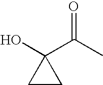 | H | CH₃ | 575 (M⁺ + 1) |
| 46 | 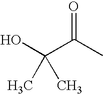 | CH₃ | H | 577 (M⁺ + 1) |

TABLE 12

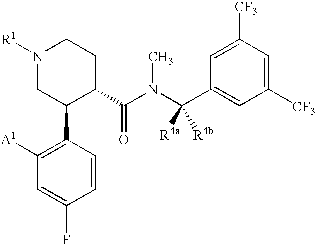

| Example | | | | | |
|---|---|---|---|---|---|
| No. | R¹ | A¹ | R^{4a} | R^{4b} | MS |
| 47 | 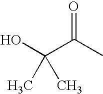 | CH₃ | H | CH₃ | 577(M⁺ + 1) |
| 48 | 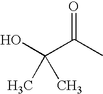 | F | CH₃ | H | 581(M⁺ + 1) |
| 49 | 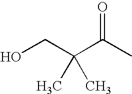 | CH₃ | CH₃ | H | 591(M⁺ + 1) |
| 50 | 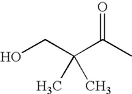 | CH₃ | H | CH₃ | 591(M⁺ + 1) |
| 51 | 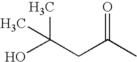 | F | CH₃ | H | 595(M⁺ + 1) |
| 52 | 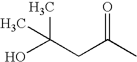 | CH₃ | H | H | 577(M⁺ + 1) |
| 53 | 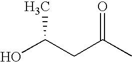 | CH₃ | CH₃ | H | 577(M⁺ + 1) |
| 54 | 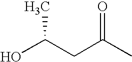 | CH₃ | H | CH₃ | 577(M⁺ + 1) |
| 55 | 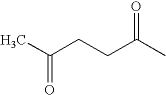 | CH₃ | CH₃ | H | 589(M⁺ + 1) |

TABLE 13

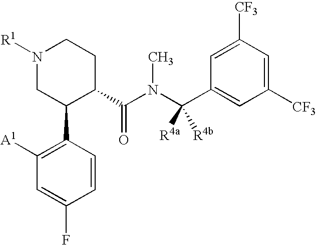

| Ex- | | | | | |
|---|---|---|---|---|---|
| am- | | | | | |
| ple | | | | | |
| No. | R¹ | A¹ | R^{4a} | R^{4b} | MS |
| 56 | 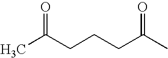 | CH₃ | CH₃ | H | 603(M⁺ + 1) |
| 57 | 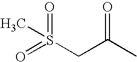 | CH₃ | CH₃ | H | 611(M⁺ + 1) |
| 58 | 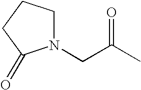 | CH₃ | CH₃ | H | 616(M⁺ + 1) |
| 59 | 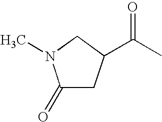 | CH₃ | CH₃ | H | 616(M⁺ + 1) |
| 60 | 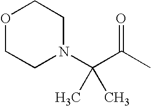 | CH₃ | CH₃ | H | 646(M⁺ + 1) |
| 61 | 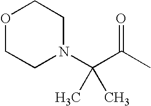 | CH₃ | H | CH₃ | 646(M⁺ + 1) |
| 62 | 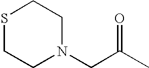 | CH₃ | CH₃ | H | 637(M⁺ + 1) |
| 63 | 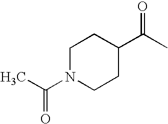 | F | CH₃ | H | 648(M⁺ + 1) |

TABLE 14

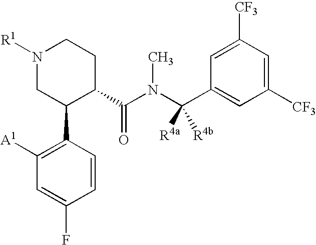

| Example | | | | | |
|---|---|---|---|---|---|
| No. | R¹ | A¹ | R^{4a} | R^{4b} | MS |
| 64 | 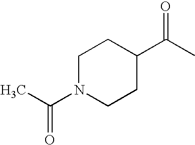 | CH₃ | H | H | 630 (M⁺ + 1) |
| 65 | 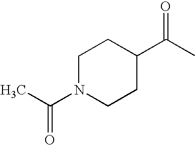 | CH₃ | CH₃ | CH₃ | 658 (M⁺ + 1) |
| 66 | 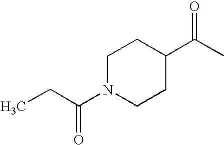 | CH₃ | CH₃ | H | 658 (M⁺ + 1) |
| 67 | 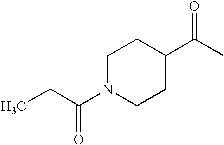 | CH₃ | H | CH₃ | 658 (M⁺ + 1) |
| 68 | 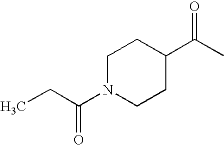 | F | CH₃ | H | 662 (M⁺ + 1) |
| 69 | 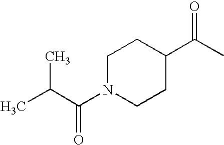 | CH₃ | CH₃ | H | 672 (M⁺ + 1) |

TABLE 15

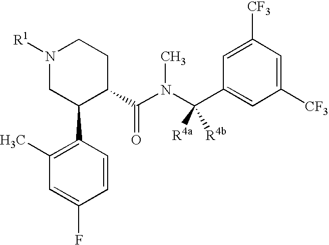

| Exam- | | | | |
|---|---|---|---|---|
| ple | | | | |
| No. | R¹ | R^{4a} | R^{4b} | MS |
| 70 | 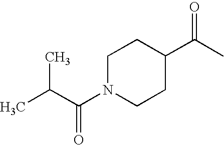 | H | CH₃ | 672(M⁺ + 1) |
| 71 | 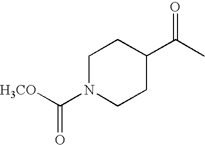 | CH₃ | H | 660(M⁺ + 1) |
| 72 | 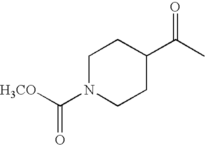 | H | CH₃ | 660(M⁺ + 1) |
| 73 | 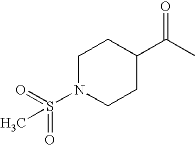 | CH₃ | H | 680(M⁺ + 1) |
| 74 | 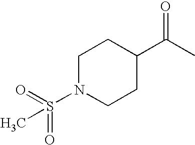 | H | CH₃ | 680(M⁺ + 1) |
| 75 | 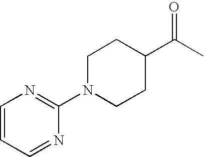 | CH₃ | H | 680(M⁺ + 1) |

TABLE 16

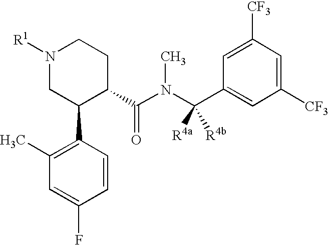

| Exam- | | | | |
|---|---|---|---|---|
| ple | | | | |
| No. | R¹ | R^{4a} | R^{4b} | MS |
| 76 | 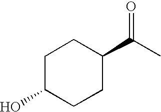 | CH₃ | H | 617(M⁺ + 1) |
| 77 | 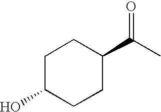 | H | CH₃ | 617(M⁺ + 1) |
| 78 | 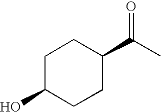 | CH₃ | H | 617(M⁺ + 1) |
| 79 | 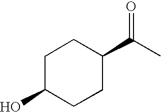 | H | CH₃ | 617(M⁺ + 1) |
| 80 | 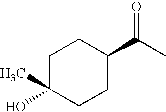 | CH₃ | H | 631(M⁺ + 1) |
| 81 | 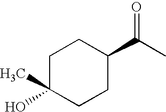 | H | CH₃ | 631(M⁺ + 1) |
| 82 | 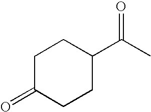 | CH₃ | H | 615(M⁺ + 1) |
| 83 | 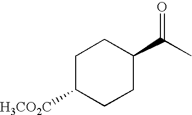 | CH₃ | H | 659(M⁺ + 1) |

TABLE 17

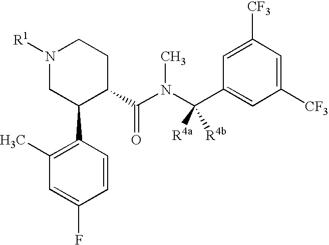

| Exam- | | | | |
|---|---|---|---|---|
| ple | | | | |
| No. | R¹ | R^{4a} | R^{4b} | MS |
| 84 | 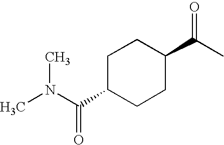 | CH₃ | H | 672(M⁺ + 1) |
| 85 | 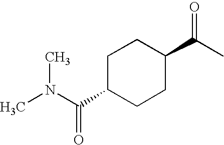 | H | CH₃ | 672(M⁺ + 1) |
| 86 | 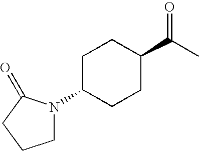 | CH₃ | H | 684(M⁺ + 1) |
| 87 | 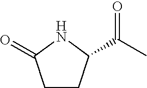 | CH₃ | H | 602(M⁺ + 1) |
| 88 | 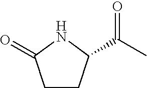 | H | CH₃ | 602(M⁺ + 1) |
| 89 | 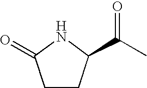 | CH₃ | H | 602(M⁺ + 1) |
| 90 | 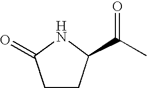 | H | CH₃ | 602(M⁺ + 1) |

TABLE 18

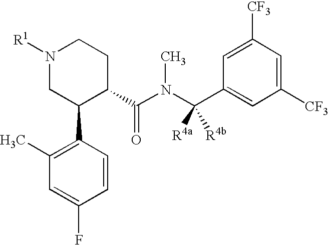

| Exam- | | | | |
|---|---|---|---|---|
| ple | | | | |
| No. | R¹ | R^{4a} | R^{4b} | MS |
| 91 | 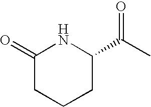 | CH₃ | H | 616(M⁺ + 1) |
| 92 | 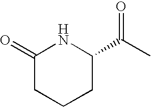 | H | CH₃ | 616(M⁺ + 1) |
| 93 | 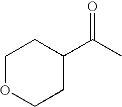 | CH₃ | H | 637(M⁺ + 1) |
| 94 | 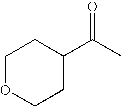 | H | CH₃ | 637(M⁺ + 1) |
| 95 | 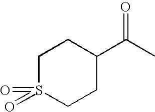 | CH₃ | H | 651(M⁺ + 1) |
| 96 | 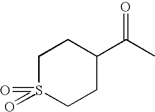 | H | CH₃ | 651(M⁺ + 1) |
| 97 | 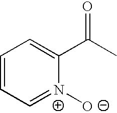 | CH₃ | H | 612(M⁺ + 1) |
| 98 | 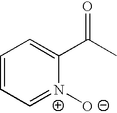 | H | CH₃ | 612(M⁺ + 1) |

TABLE 19

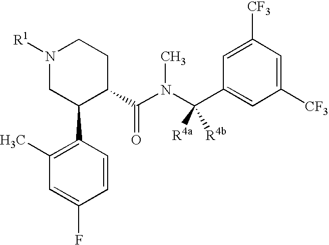

| Exam- | | | | |
|---|---|---|---|---|
| ple | | | | |
| No. | R¹ | R^{4a} | R^{4b} | MS |
| 99 | 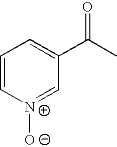 | CH₃ | H | 612(M⁺ + 1) |
| 100 | 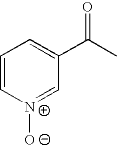 | H | CH₃ | 612(M⁺ + 1) |
| 101 | 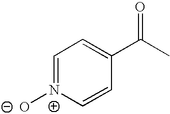 | CH₃ | H | 612(M⁺ + 1) |
| 102 | 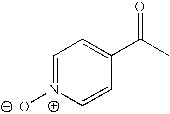 | H | CH₃ | 612(M⁺ + 1) |
| 103 | 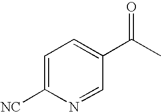 | CH₃ | H | 621(M⁺ + 1) |
| 104 | 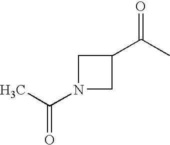 | H | CH₃ | 616(M⁺ + 1) |
| 105 | 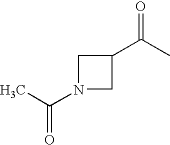 | CH₃ | H | 616(M⁺ + 1) |

TABLE 20

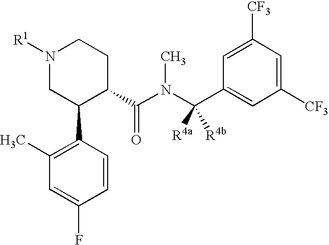

| Exam- | | | | |
|---|---|---|---|---|
| ple | | | | |
| No. | R¹ | R^{4a} | R^{4b} | MS |
| 106 | 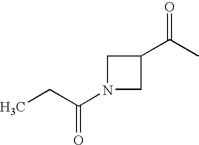 | CH₃ | H | 630(M⁺ + 1) |
| 107 | 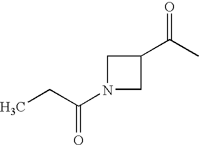 | H | CH₃ | 630(M⁺ + 1) |
| 108 | 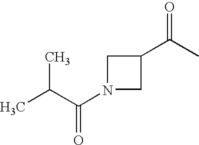 | CH₃ | H | 644(M⁺ + 1) |
| 109 | 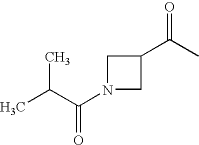 | H | CH₃ | 644(M⁺ + 1) |
| 110 | 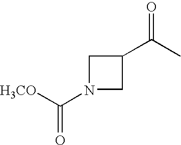 | CH₃ | H | 632(M⁺ + 1) |
| 111 | 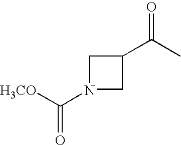 | H | CH₃ | 632(M⁺ + 1) |

TABLE 21

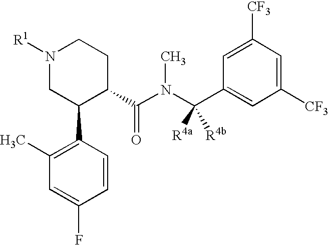

| Exam- | | | | |
|---|---|---|---|---|
| ple | | | | |
| No. | R¹ | R^{4a} | R^{4b} | MS |
| 112 | 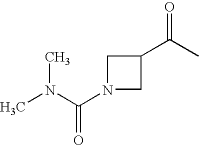 | CH₃ | H | 645(M⁺ + 1) |
| 113 | 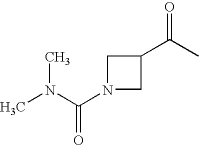 | H | CH₃ | 645(M⁺ + 1) |
| 114 | 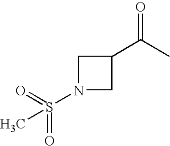 | CH₃ | H | 652(M⁺ + 1) |
| 115 | 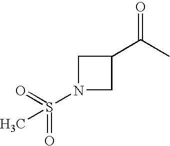 | H | CH₃ | 652(M⁺ + 1) |

TABLE 22

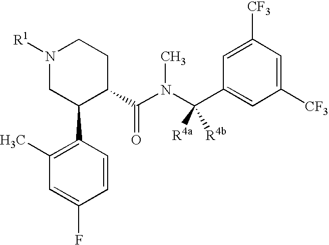

| Exam- | | | | |
|---|---|---|---|---|
| ple | | | | |
| No. | R¹ | R^{4a} | R^{4b} | MS |
| 116 | 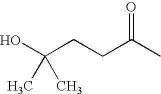 | CH₃ | H | 605(M⁺ + 1) |
| 117 | 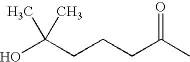 | CH₃ | H | 619(M⁺ + 1) |
| 118 | 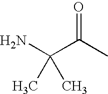 | CH₃ | H | 576(M⁺ + 1) |
| 119 | 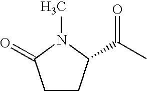 | CH₃ | H | 616(M⁺ + 1) |
| 120 | 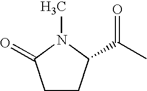 | H | CH₃ | 616(M⁺ + 1) |
| 121 | 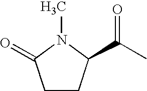 | CH₃ | H | 616(M⁺ + 1) |
| 122 | 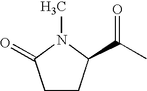 | H | CH₃ | 616(M⁺ + 1) |
| 123 | 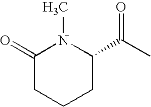 | CH₃ | H | 630(M⁺ + 1) |
| 124 | 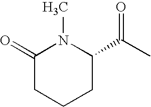 | H | CH₃ | 630(M⁺ + 1) |

TABLE 23

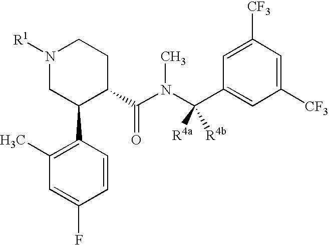

| Exam- | | | | |
|---|---|---|---|---|
| ple | | | | |
| No. | R¹ | R^{4a} | R^{4b} | MS |
| 125 | 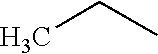 | CH₃ | H | 519(M⁺ + 1) |
| 126 | 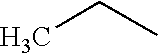 | H | CH₃ | 519(M⁺ + 1) |
| 127 | 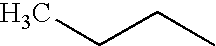 | CH₃ | H | 533(M⁺ + 1) |
| 128 | 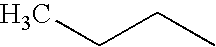 | H | CH₃ | 533(M⁺ + 1) |
| 129 | 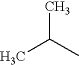 | CH₃ | H | 533(M⁺ + 1) |
| 130 | 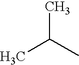 | H | CH₃ | 533(M⁺ + 1) |
| 131 | 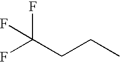 | CH₃ | H | 587(M⁺ + 1) |
| 132 | 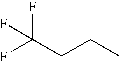 | H | CH₃ | 587(M⁺ + 1) |
| 133 | 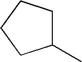 | CH₃ | H | 559(M⁺ + 1) |
| 134 | 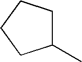 | H | CH₃ | 559(M⁺ + 1) |
| 135 | 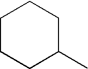 | CH₃ | H | 573(M⁺ + 1) |
| 136 | 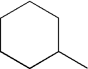 | H | CH₃ | 573(M⁺ + 1) |
| 137 | 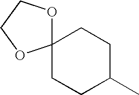 | CH₃ | H | 631(M⁺ + 1) |
| 138 | 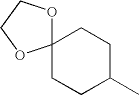 | H | CH₃ | 631(M⁺ + 1) |

TABLE 24

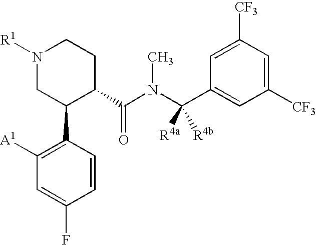

| Exam- | | | | | |
|---|---|---|---|---|---|
| ple | | | | | |
| No. | R¹ | A¹ | R^{4a} | R^{4b} | MS |
| 139 | 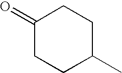 | CH₃ | CH₃ | H | 587(M⁺ + 1) |
| 140 | 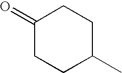 | CH₃ | H | CH₃ | 587(M⁺ + 1) |
| 141 | 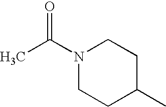 | F | CH₃ | H | 620(M⁺ + 1) |
| 142 | 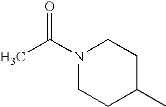 | CH₃ | H | H | 602(M⁺ + 1) |
| 143 | 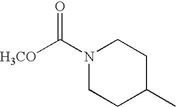 | CH₃ | CH₃ | H | 632(M⁺ + 1) |
| 144 | 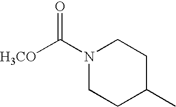 | CH₃ | H | CH₃ | 632(M⁺ + 1) |
| 145 | 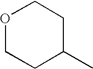 | CH₃ | CH₃ | H | 575(M⁺ + 1) |
| 146 | 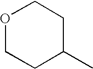 | CH₃ | H | CH₃ | 575(M⁺ + 1) |
| 147 | 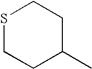 | CH₃ | CH₃ | H | 591(M⁺ + 1) |
| 148 | 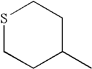 | CH₃ | H | CH₃ | 591(M⁺ + 1) |
| 149 | 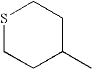 | CH₃ | CH₃ | CH₃ | 605(M⁺ + 1) |

TABLE 25

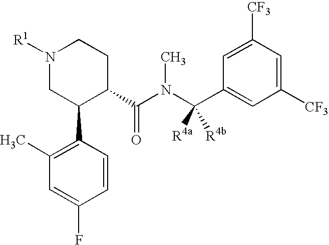

| Exam- | | | | |
|---|---|---|---|---|
| ple | | | | |
| No. | R¹ | R^{4a} | R^{4b} | MS |
| 150 | 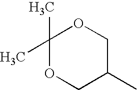 | CH₃ | H | 605(M⁺ + 1) |
| 151 | 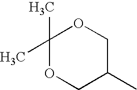 | H | CH₃ | 605(M⁺ + 1) |
| 152 | 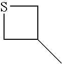 | CH₃ | H | 563(M⁺ + 1) |
| 153 | 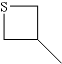 | H | CH₃ | 563(M⁺ + 1) |
| 154 | 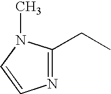 | CH₃ | H | 585(M⁺ + 1) |
| 155 | 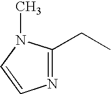 | H | CH₃ | 585(M⁺ + 1) |
| 156 | 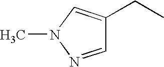 | CH₃ | H | 585(M⁺ + 1) |
| 157 | 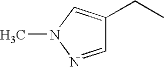 | H | CH₃ | 585(M⁺ + 1) |
| 158 | 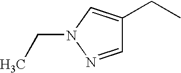 | CH₃ | H | 599(M⁺ + 1) |

TABLE 26

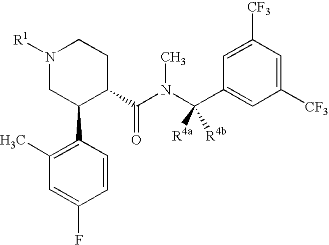

| Exam- | | | | |
|---|---|---|---|---|
| ple | | | | |
| No. | R¹ | R^{4a} | R^{4b} | MS |
| 159 | 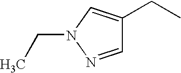 | H | CH₃ | 599(M⁺ + 1) |
| 160 | 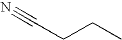 | CH₃ | H | 544(M⁺ + 1) |
| 161 | 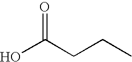 | CH₃ | H | 563(M⁺ + 1) |
| 162 | 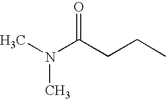 | CH₃ | H | 590(M⁺ + 1) |
| 163 | 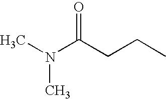 | H | CH₃ | 590(M⁺ + 1) |
| 164 | 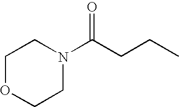 | CH₃ | H | 632(M⁺ + 1) |
| 165 | 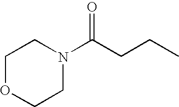 | H | CH₃ | 632(M⁺ + 1) |
| 166 | 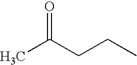 | CH₃ | H | 561(M⁺ + 1) |
| 167 | 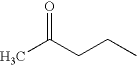 | H | CH₃ | 561(M⁺ + 1) |
| 168 | 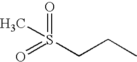 | CH₃ | H | 597(M⁺ + 1) |

TABLE 27

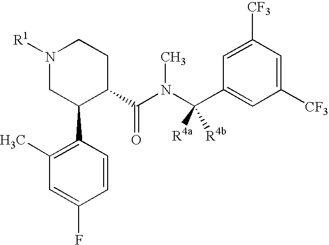

| Exam- | | | | |
|---|---|---|---|---|
| ple | | | | |
| No. | R¹ | R^{4a} | R^{4b} | MS |
| 169 | 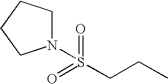 | CH₃ | H | 652(M⁺ + 1) |
| 170 | 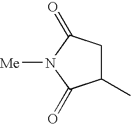 | CH₃ | H | 602(M⁺ + 1) |
| 171 | 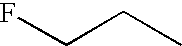 | CH₃ | H | 537(M⁺ + 1) |
| 172 | 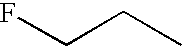 | H | CH₃ | 537(M⁺ + 1) |
| 173 | 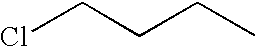 | CH₃ | H | 567(M⁺ + 1) |
| 174 | 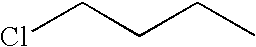 | H | CH₃ | 567(M⁺ + 1) |
| 175 | 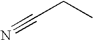 | CH₃ | H | 530(M⁺ + 1) |
| 176 | 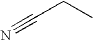 | H | CH₃ | 530(M⁺ + 1) |
| 177 | 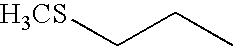 | CH₃ | H | 530(M⁺ + 1) |
| 178 | 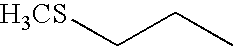 | H | CH₃ | 565(M⁺ + 1) |
| 179 | 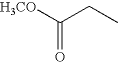 | CH₃ | H | 563(M⁺ + 1) |

TABLE 28

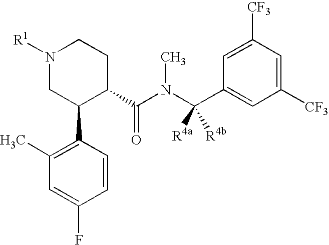

| Exam- | | | | |
|---|---|---|---|---|
| ple | | | | |
| No. | R¹ | R^{4a} | R^{4b} | MS |
| 180 | 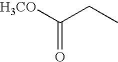 | H | CH₃ | 563(M⁺ + 1) |
| 181 | 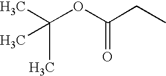 | CH₃ | H | 605(M⁺ + 1) |
| 182 | 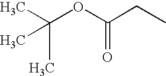 | H | CH₃ | 605(M⁺ + 1) |
| 183 | 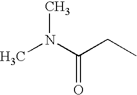 | CH₃ | H | 576(M⁺ + 1) |
| 184 | 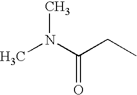 | H | CH₃ | 576(M⁺ + 1) |
| 185 | 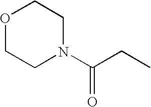 | CH₃ | H | 618(M⁺ + 1) |
| 186 | 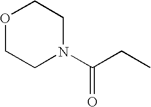 | H | CH₃ | 618(M⁺ + 1) |
| 187 | 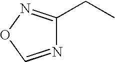 | CH₃ | H | 573(M⁺ + 1) |
| 188 | 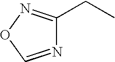 | H | CH₃ | 573(M⁺ + 1) |

TABLE 29

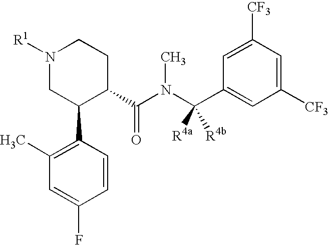

| Exam- | | | | |
|---|---|---|---|---|
| ple | | | | |
| No. | R¹ | R^{4a} | R^{4b} | MS |
| 189 | 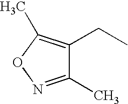 | CH₃ | H | 600(M⁺ + 1) |
| 190 | 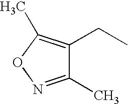 | H | CH₃ | 600(M⁺ + 1) |
| 191 | 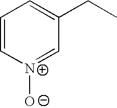 | CH₃ | H | 598(M⁺ + 1) |
| 192 | 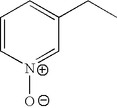 | H | CH₃ | 598(M⁺ + 1) |
| 193 | 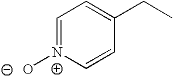 | CH₃ | H | 598(M⁺ + 1) |
| 194 | 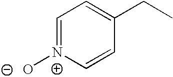 | H | CH₃ | 598(M⁺ + 1) |
| 195 | 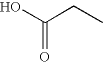 | CH₃ | H | 549(M⁺ + 1) |
| 196 | 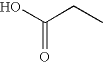 | H | CH₃ | 549(M⁺ + 1) |

TABLE 30

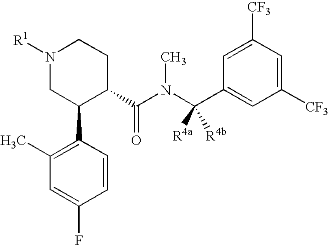

| Example | | | | |
|---|---|---|---|---|
| No. | R¹ | R^{4a} | R^{4b} | MS |
| 197 | 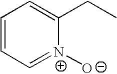 | CH₃ | H | 598 (M⁺ + 1) |
| 198 | 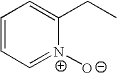 | H | CH₃ | 598 (M⁺ + 1) |
| 199 | 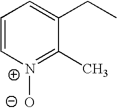 | CH₃ | H | 612 (M⁺ + 1) |
| 200 | 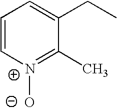 | H | CH₃ | 612 (M⁺ + 1) |
| 201 | 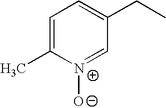 | CH₃ | H | 612 (M⁺ + 1) |
| 202 | 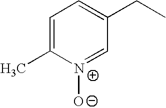 | H | CH₃ | 612 (M⁺ + 1) |
| 203 | 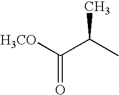 | CH₃ | H | 577 (M⁺ + 1) |
| 204 | 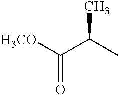 | H | CH₃ | 577 (M⁺ + 1) |
| 205 | 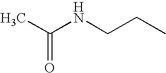 | H | CH₃ | 576 (M⁺ + 1) |

TABLE 31

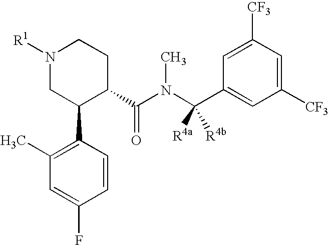

| Ex- | | | | |
|---|---|---|---|---|
| ample | | | | |
| No. | R¹ | R^{4a} | R^{4b} | MS |
| 206 | 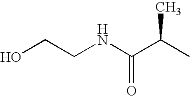 | CH₃ | H | 606 (M⁺ + 1) |
| 207 | 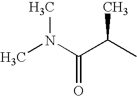 | CH₃ | H | 590 (M⁺ + 1) |
| 208 | 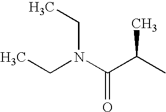 | H | CH₃ | 618 (M⁺ + 1) |
| 209 | 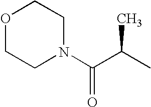 | CH₃ | H | 632 (M⁺ + 1) |
| 210 | 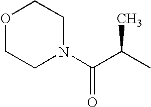 | H | CH₃ | 632 (M⁺ + 1) |
| 211 | 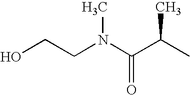 | CH₃ | H | 620 (M⁺ + 1) |
| 212 | 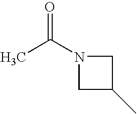 | H | CH₃ | 588 (M⁺ + 1) |
| 213 | 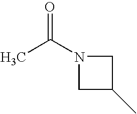 | CH₃ | H | 588 (M⁺ + 1) |

TABLE 32

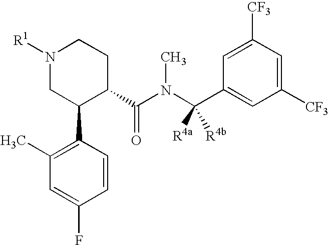

| Example | | | | |
|---|---|---|---|---|
| No. | R¹ | R^{4a} | R^{4b} | MS |
| 214 | 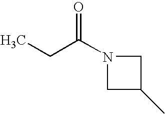 | CH₃ | H | 602 (M⁺ + 1) |
| 215 | 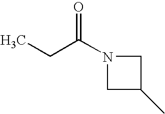 | H | CH₃ | 602 (M⁺ + 1) |
| 216 | 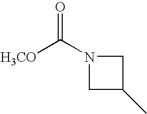 | CH₃ | H | 604 (M⁺ + 1) |
| 217 | 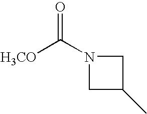 | H | CH₃ | 604 (M⁺ + 1) |
| 218 | 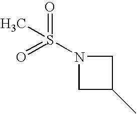 | CH₃ | H | 624 (M⁺ + 1) |
| 219 | 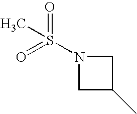 | H | CH₃ | 624 (M⁺ + 1) |
| 220 | 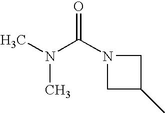 | CH₃ | H | 617 (M⁺ + 1) |
| 221 | 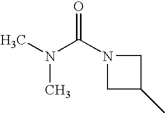 | H | CH₃ | 617 (M⁺ + 1) |

TABLE 33

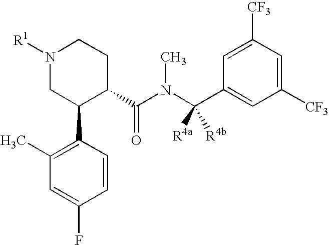

| Example | | | | |
|---|---|---|---|---|
| No. | R¹ | R^{4a} | R^{4b} | MS |
| 222 | 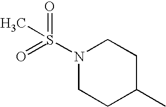 | CH₃ | H | 652 (M⁺ + 1) |
| 223 | 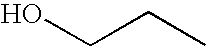 | CH₃ | H | 535 (M⁺ + 1) |
| 224 | 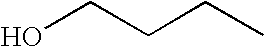 | CH₃ | H | 549 (M⁺ + 1) |
| 225 | 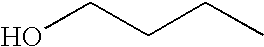 | H | CH₃ | 549 (M⁺ + 1) |
| 226(a) | 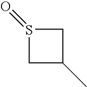 | CH₃ | H | 579 (M⁺ + 1) |
| 226(b) | 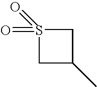 | CH₃ | H | 595 (M⁺ + 1) |
| 227(a) | 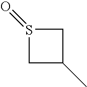 | H | CH₃ | 579 (M⁺ + 1) |
| 227(b) | 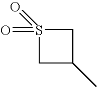 | H | CH₃ | 595 (M⁺ + 1) |
| 228 | 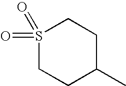 | CH₃ | H | 623 (M⁺ + 1) |

TABLE 34

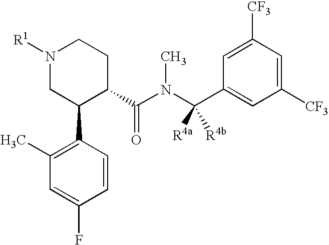

| Example | | | | |
|---|---|---|---|---|
| No. | R¹ | R^{4a} | R^{4b} | MS |
| 229 | 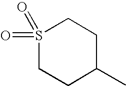 | H | CH₃ | 623 (M⁺ + 1) |
| 230 | 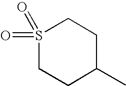 | CH₃ | CH₃ | 637 (M⁺ + 1) |
| 231 | 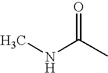 | CH₃ | H | 548 (M⁺ + 1) |
| 232 | 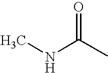 | H | CH₃ | 548 (M⁺ + 1) |
| 233 | 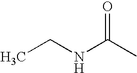 | CH₃ | H | 562 (M⁺ + 1) |
| 234 | 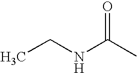 | H | CH₃ | 562 (M⁺ + 1) |
| 235 | 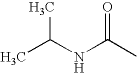 | CH₃ | H | 576 (M⁺ + 1) |
| 236 | 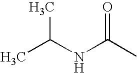 | H | CH₃ | 576 (M⁺ + 1) |

TABLE 35

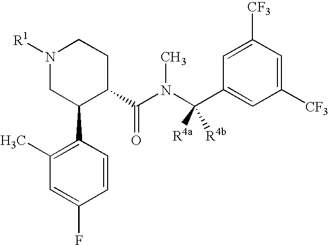

| Example | | | | |
|---|---|---|---|---|
| No. | R¹ | R^{4a} | R^{4b} | MS |
| 237 | 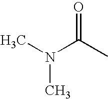 | CH₃ | H | 562 (M⁺ + 1) |
| 238 | 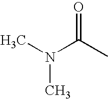 | H | CH₃ | 562 (M⁺ + 1) |
| 239 | 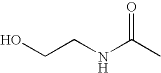 | CH₃ | H | 578 (M⁺ + 1) |
| 240 | 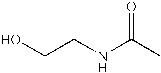 | H | CH₃ | 578 (M⁺ + 1) |
| 241 | 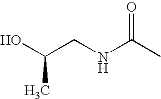 | CH₃ | H | 592 (M⁺ + 1) |
| 242 | 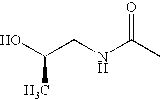 | H | CH₃ | 592 (M⁺ + 1) |
| 243 | 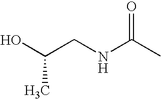 | CH₃ | H | 592 (M⁺ + 1) |
| 244 | 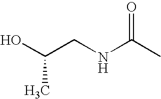 | H | CH₃ | 592 (M⁺ + 1) |

TABLE 36

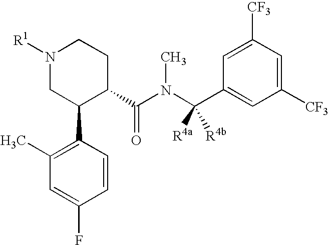

| Example | | | | |
|---|---|---|---|---|
| No. | R¹ | R^{4a} | R^{4b} | MS |
| 245 | 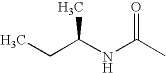 | H | CH₃ | 592 (M⁺ + 1) |
| 246 | 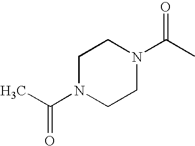 | CH₃ | H | 645 (M⁺ + 1) |
| 247 | 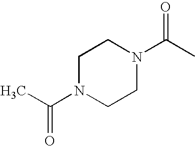 | H | CH₃ | 645 (M⁺ + 1) |
| 248 | 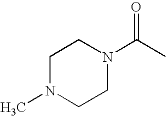 | CH₃ | H | 617 (M⁺ + 1) |
| 249 | 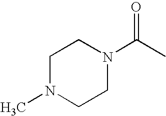 | H | CH₃ | 617 (M⁺ + 1) |
| 250 | 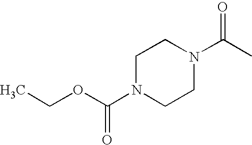 | CH₃ | H | 675 (M⁺ + 1) |
| 251 | 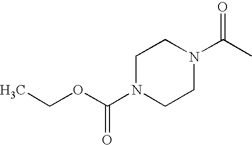 | H | CH₃ | 675 (M⁺ + 1) |

TABLE 37

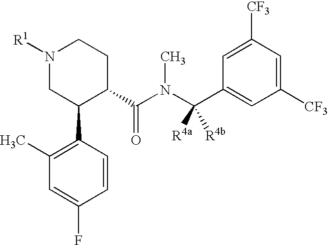

| Ex- | | | | |
|---|---|---|---|---|
| ample | | | | |
| No. | R¹ | R^{4a} | R^{4b} | MS |
| 252 | 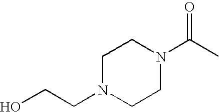 | CH₃ | H | 647(M⁺ + 1) |
| 253 | 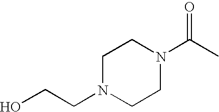 | H | CH₃ | 647(M⁺ + 1) |
| 254 | 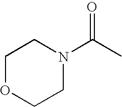 | CH₃ | H | 604(M⁺ + 1) |
| 255 | 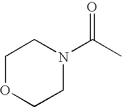 | H | CH₃ | 604(M⁺ + 1) |
| 256 | 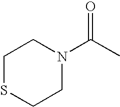 | CH₃ | H | 620(M⁺ + 1) |
| 257 | 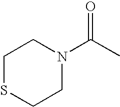 | H | CH₃ | 620(M⁺ + 1) |
| 258 | 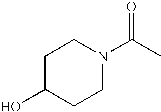 | CH₃ | H | 618(M⁺ + 1) |
| 259 | 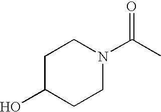 | H | CH₃ | 618(M⁺ + 1) |

TABLE 38

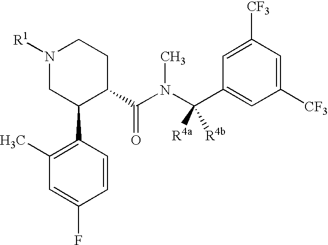

| Example | | | | |
|---|---|---|---|---|
| No. | R¹ | R^{4a} | R^{4b} | MS |
| 260 | 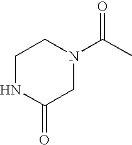 | CH₃ | H | 617 (M⁺ + 1) |
| 261 | 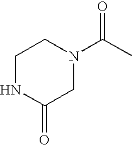 | H | CH₃ | 617 (M⁺ + 1) |
| 262 | 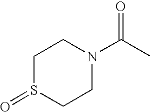 | CH₃ | H | 636 (M⁺ + 1) |
| 263 | 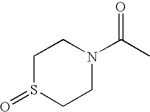 | H | CH₃ | 636 (M⁺ + 1) |
| 264 | 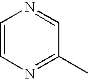 | CH₃ | H | 569 (M⁺ + 1) |
| 265 | 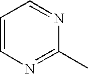 | CH₃ | H | 569 (M⁺ + 1) |
| 266 | 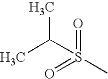 | CH₃ | H | 597 (M⁺ + 1) |
| 267 | 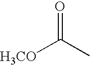 | CH₃ | H | 549 (M⁺ + 1) |
| 268 | 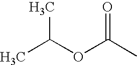 | CH₃ | H | 577 (M⁺ + 1) |

TABLE 39

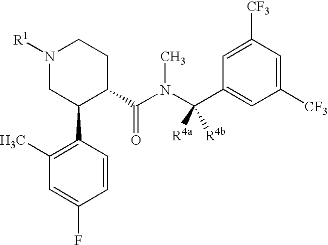

| Example | | | | |
|---|---|---|---|---|
| No. | R¹ | R^{4a} | R^{4b} | MS |
| 269 | 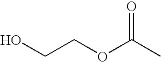 | H | CH₃ | 579 (M⁺ + 1) |
| 270 | 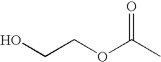 | CH₃ | H | 579 (M⁺ + 1) |
| 271 | 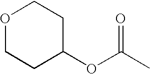 | CH₃ | H | 619 (M⁺ + 1) |
| 272 | 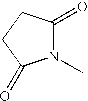 | CH₃ | H | 588 (M⁺ + 1) |
| 273 | 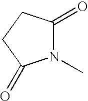 | H | CH₃ | 588 (M⁺ + 1) |
| 274 | 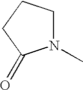 | CH₃ | H | 574 (M⁺ + 1) |
| 275 | 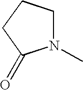 | H | CH₃ | 574 (M⁺ + 1) |
| 276 | 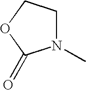 | CH₃ | H | 576 (M⁺ + 1) |
| 277 | 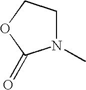 | H | CH₃ | 576 (M⁺ + 1) |

TABLE 40

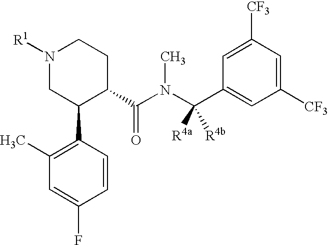

| Ex- | | | | |
|---|---|---|---|---|
| ample | | | | |
| No. | R¹ | R^{4a} | R^{4b} | MS |
| 278 | 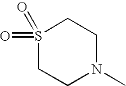 | CH₃ | H | 624 (M⁺ + 1) |
| 279 | 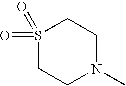 | H | CH₃ | 624 (M⁺ + 1) |
| 280 | 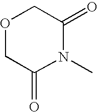 | CH₃ | H | 604 (M⁺ + 1) |
| 281 | 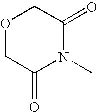 | H | CH₃ | 604 (M⁺ + 1) |
| 282 | 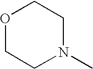 | CH₃ | H | 576 (M⁺ + 1) |
| 283 | 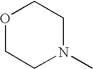 | H | CH₃ | 576 (M⁺ + 1) |
| 284 | 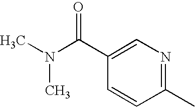 | CH₃ | H | 639 (M⁺ + 1) |
| 285 | 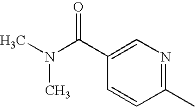 | H | CH₃ | 639 (M⁺ + 1) |
| 286 | 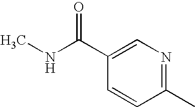 | CH₃ | H | 625 (M⁺ + 1) |
| 287 | 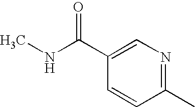 | H | CH₃ | 625 (M⁺ + 1) |

TABLE 41

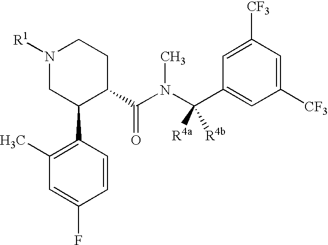

| Example | | | | |
|---|---|---|---|---|
| No. | R¹ | R^{4a} | R^{4b} | MS |
| 288 | 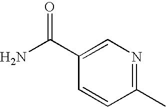 | CH₃ | H | 611 (M⁺ + 1) |
| 289 | 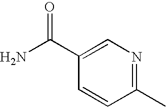 | H | CH₃ | 611 (M⁺ + 1) |
| 290 | 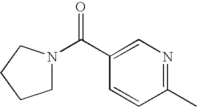 | CH₃ | H | 665 (M⁺ + 1) |
| 291 | 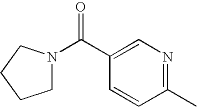 | H | CH₃ | 665 (M⁺ + 1) |
| 292 | 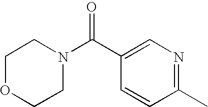 | CH₃ | H | 681 (M⁺ + 1) |
| 293 | 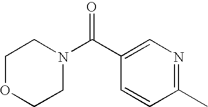 | H | CH₃ | 681 (M⁺ + 1) |
| 294 | 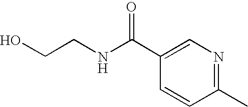 | CH₃ | H | 655 (M⁺ + 1) |
| 295 | 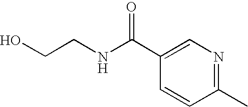 | H | CH₃ | 655 (M⁺ + 1) |
| 296 | 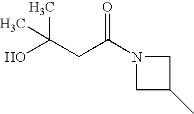 | CH₃ | H | 646 (M⁺ + 1) |

TABLE 42

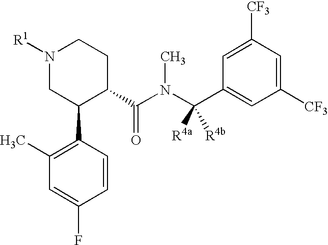

| Example | | | | |
|---|---|---|---|---|
| No. | R¹ | R^{4a} | R^{4b} | MS |
| 297 | 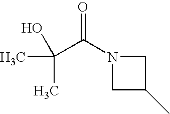 | CH₃ | H | 632 (M⁺ + 1) |
| 298 | 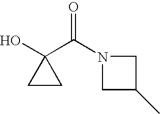 | CH₃ | H | 630 (M⁺ + 1) |
| 299 | 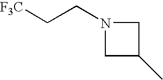 | CH₃ | H | 642 (M⁺ + 1) |

TABLE 43

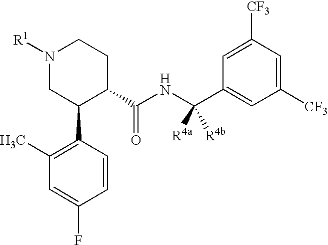

| Example | | | | |
|---|---|---|---|---|
| No. | R¹ | R^{4a} | R^{4b} | MS |
| 300(a) | 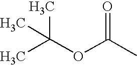 | C₂H₅ | H | 491 (M⁺ + 2-Boc) |
| 300(b) | 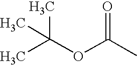 | H | C₂H₅ | 491 (M⁺ + 2-Boc) |
| 301(a) | 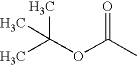 | 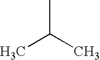 | H | 505 (M⁺ + 2-Boc) |
| 301(b) | 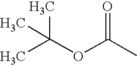 | H | 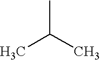 | 505 (M⁺ + 2-Boc) |

The “Boc” represents tert-butoxycarbonyl moiety.

TABLE 44

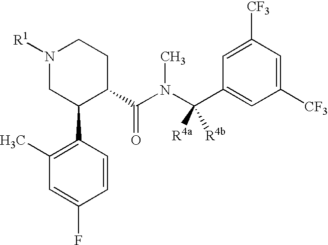

| Example | | | | |
|---|---|---|---|---|
| No. | R¹ | R^{4a} | R^{4b} | MS |
| 302 | 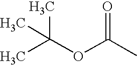 | C₂H₅ | H | 505 (M⁺ + 2-Boc) |
| 303 | 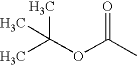 | H | C₂H₅ | 505 (M⁺ + 2-Boc) |
| 304 | 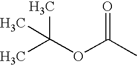 | 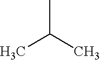 | H | 519 (M⁺ + 2-Boc) |
| 305 | 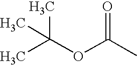 | H | 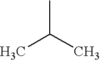 | 519 (M⁺ + 2-Boc) |
| 306 | H | C₂H₅ | H | 505 (M⁺ + 1) |
| 307 | H | H | C₂H₅ | 505 (M⁺ + 1) |
| 308 | H | 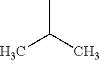 | H | 519 (M⁺ + 1) |
| 309 | H | H | 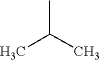 | 519 (M⁺ + 1) |
| 310 | 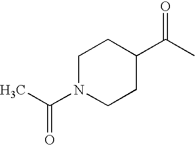 | C₂H₅ | H | 658 (M⁺ + 1) |
| 311 | 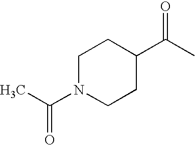 | H | C₂H₅ | 658 (M⁺ + 1) |

The “Boc” represents tert-butoxycarbonyl moiety.

TABLE 45

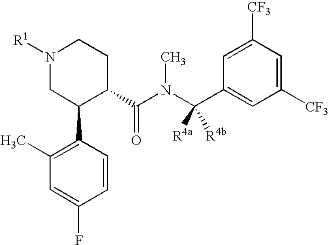

| Example | | | | |
|---|---|---|---|---|
| No. | R¹ | R^{4a} | R^{4b} | MS |
| 312 | 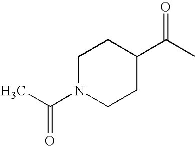 | 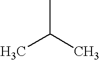 | H | 672 (M⁺ + 1) |
| 313 | 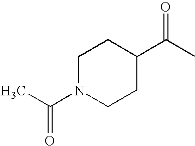 | H | 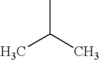 | 672 (M⁺ + 1) |
| 314 | 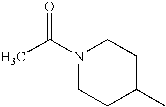 | C₂H₅ | H | 630 (M⁺ + 1) |
| 315 | 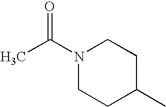 | H | C₂H₅ | 630 (M⁺ + 1) |
| 316 | 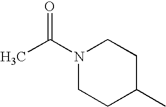 | 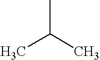 | H | 644 (M⁺ + 1) |
| 317 | 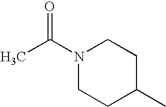 | H | 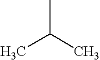 | 644 (M⁺ + 1) |
| 318 | 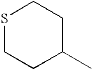 | C₂H₅ | H | 605 (M⁺ + 1) |
| 319 | 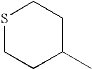 | H | C₂H₅ | 605 (M⁺ + 1) |
| 320 | 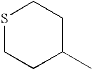 | H | 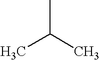 | 619 (M⁺ + 1) |

TABLE 46

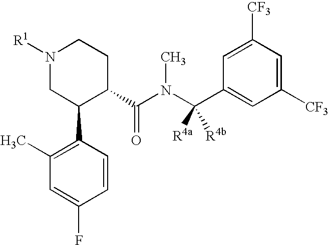

| Example | | | | |
|---|---|---|---|---|
| No. | R¹ | R^{4a} | R^{4b} | MS |
| 321 | 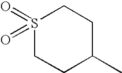 | C₂H₅ | H | 637 (M⁺ + 1) |
| 322 | 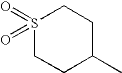 | H | C₂H₅ | 637 (M⁺ + 1) |
| 323 | 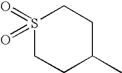 | H | 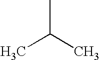 | 637 (M⁺ + 1) |

TABLE 47

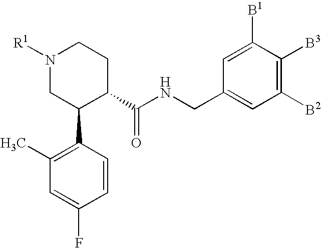

| Example | | | | | |
|---|---|---|---|---|---|
| No. | R¹ | B¹ | B² | B³ | MS |
| 324 | 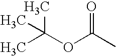 | CH₃ | CH₃ | H | 455 (M⁺ + 1) |
| 325 | 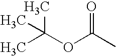 | Cl | Cl | H | 395, 397(M⁺ + 2-Boc) |
| 326 | 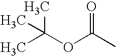 | Cl | H | Cl | 395, 397(M⁺ + 2-Boc) |
| 327 | 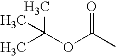 | OCH₃ | OCH₃ | H | 487 (M⁺ + 1) |

The “Boc” represents tert-butoxycarbonyl moiety.

TABLE 48

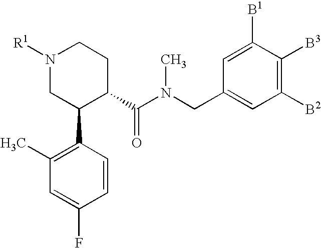

| Example | | | | | |
|---|---|---|---|---|---|
| No. | R¹ | B¹ | B² | B³ | MS |
| 328 | 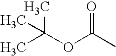 | CH₃ | CH₃ | H | 469 (M⁺ + 1) |
| 329 | 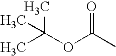 | Cl | Cl | H | 509, 511(M⁺ + 1) |
| 330 | 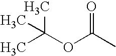 | Cl | H | Cl | 409, 411(M⁺ + 2-Boc) |
| 331 | 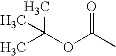 | OCH₃ | OCH₃ | H | 501 (M⁺ + 1) |
| 332 | H | CH₃ | CH₃ | H | 369 (M⁺ + 1) |
| 333 | H | Cl | Cl | H | 409, 411 |
| | | | | | (M⁺ + 1) |
| 334 | H | Cl | H | Cl | 409, 411 |
| | | | | | (M⁺ + 1) |
| 335 | H | OCH₃ | OCH₃ | H | 401 (M+ + 1) |

The “Boc” represents tertbutoxycarbonyl moiety.

TABLE 49

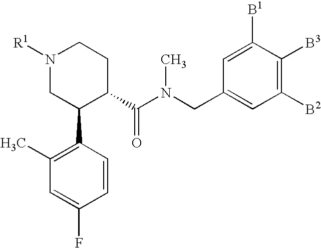

| Example | | | | | |
|---|---|---|---|---|---|
| No. | R¹ | B¹ | B² | B³ | MS |
| 336 | 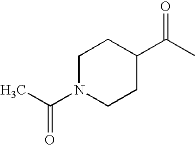 | CH₃ | CH₃ | H | 522 (M⁺ + 1) |
| 337 | 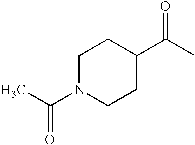 | Cl | H | Cl | 562, 564(M⁺ + 1) |
| 338 | 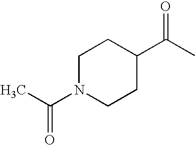 | OCH₃ | OCH₃ | H | 554 (M⁺ + 1) |
| 339 | 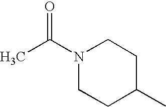 | CH₃ | CH₃ | H | 494 (M⁺ + 1) |
| 340 | 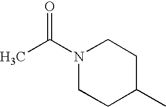 | Cl | H | Cl | 534, 535(M⁺ + 1) |
| 341 | 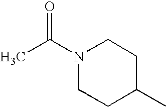 | OCH₃ | OCH₃ | H | 526 (M⁺ + 1) |

TABLE 50

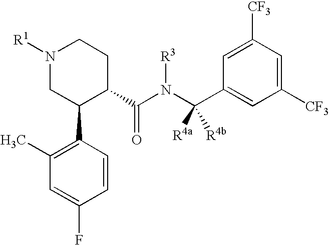

| Example | | | | | |
|---|---|---|---|---|---|
| No. | R¹ | R³ | R^{4a} | R^{4b} | MS |
| 342 | 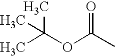 | H | H | H | 463 (M⁺ + 2-Boc) |
| 343(a) | 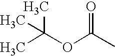 | H | CH₃ | H | 477 (M⁺ + 2-Boc) |
| 343(b) | 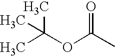 | H | H | CH₃ | 477 (M⁺ + 2-Boc) |
| 344 | 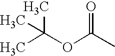 | C₂H₅ | H | H | 491 (M⁺ + 2-Boc) |
| 345 | 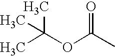 | C₂H₅ | CH₃ | H | 505 (M⁺ + 2-Boc) |
| 346 | H | C₂H₅ | H | H | 491 (M⁺ + 1) |
| 347 | H | C₂H₅ | CH₃ | H | 505 (M⁺ + 1) |
| 348 | H | H | CH₃ | H | 505 (M⁺ + 1) |
| 349 | H | H | H | CH₃ | 477 (M⁺ + 1) |

The “Boc” represents tert-butoxycarbonyl moiety.

TABLE 51

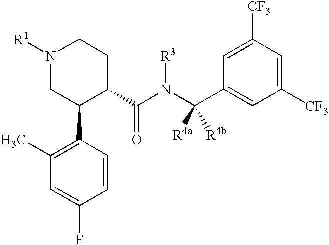

| Example | | | | | |
|---|---|---|---|---|---|
| No. | R¹ | R³ | R^{4a} | R^{4b} | MS |
| 350 | 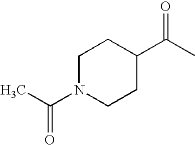 | C₂H₅ | H | H | 644 (M⁺ + 1) |
| 351 | 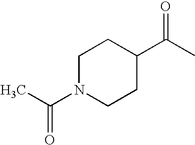 | C₂H₅ | CH₃ | H | 658 (M⁺ + 1) |
| 352 | 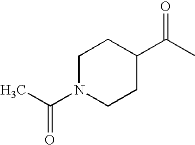 | H | CH₃ | H | 630 (M⁺ + 1) |
| 353 | 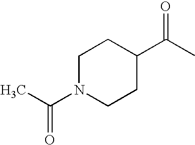 | H | H | CH₃ | 630 (M⁺ + 1) |
| 354 | 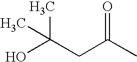 | C₂H₅ | CH₃ | H | 605 (M⁺ + 1) |
| 355 | 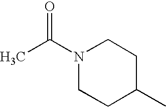 | C₂H₅ | H | H | 616 (M⁺ + 1) |
| 356 | 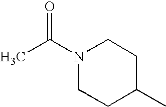 | C₂H₅ | CH₃ | H | 630 (M⁺ + 1) |

TABLE 52

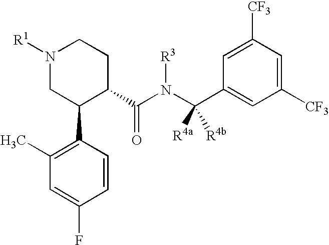

| Example | | | | | |
|---|---|---|---|---|---|
| No. | R¹ | R³ | R^{4a} | R^{4b} | MS |
| 357 | 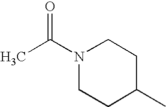 | H | CH₃ | H | 602(M⁺ + 1) |
| 358 | 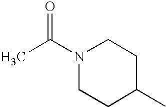 | H | H | CH₃ | 602(M⁺ + 1) |
| 359 | 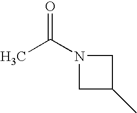 | H | H | CH₃ | 590(M⁺ + 1) |

TABLE 53

| Reference | | |
|---|---|---|
| Example | | |
| No. | Structural formula | MS |
| 1(1) | 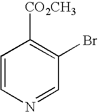 | 216, 218(M⁺ + 1) |
| 1(2) | 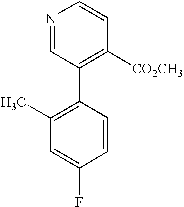 | 246 (M⁺ + 1) |
| 1(3) | 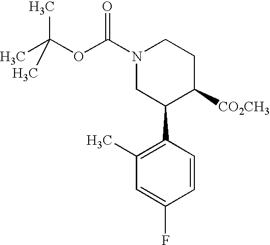 | 352 (M⁺ + 1) |
| | and | |
| | 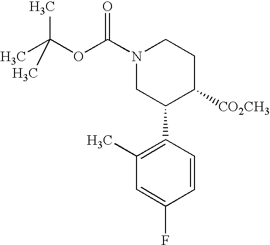 | |
| 1(4) | 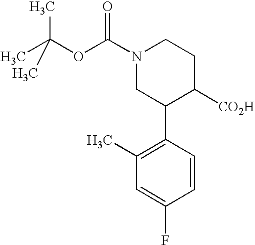 | 336 (M⁺ − 1) |

TABLE 54

| Reference | | |
|---|---|---|
| Example No. | Structural formula | MS |
| 1(5) | 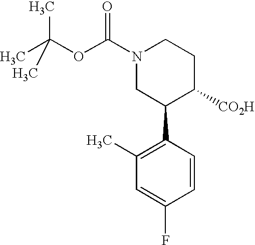 | 336 (M⁺ − 1) |
| | and | |
| | 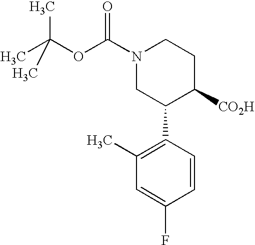 | |
| 1(6)(a) | 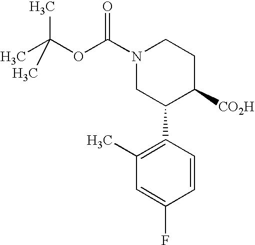 | 336 (M⁺ + 1) |
| 1(6)(b) | 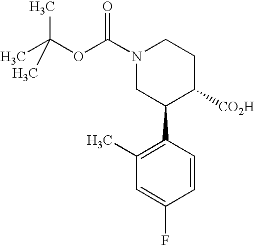 | 336 (M⁺ + 1) |

TABLE 55

| Reference | | |
|---|---|---|
| Example | | |
| No. | Structural formula | MS |
| 2(1) | 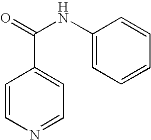 | 199 (M⁺ + 1) |
| 2(2) | 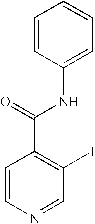 | 325 (M⁺ + 1) |
| 2(3) | 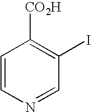 | 250 (M⁺ + 1) |
| 2(4) | 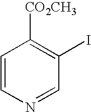 | 264 (M⁺ + 1) |
| 2(5) | 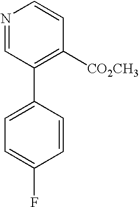 | 232 (M⁺ + 1) |
| 2(6) | 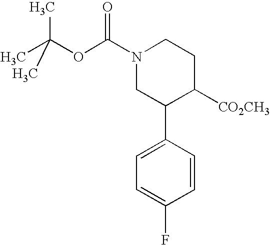 | 338 (M⁺ + 1) |

TABLE 56

| Reference | | |
|---|---|---|
| Example | | |
| No. | Structural formula | MS |
| 2(7) | 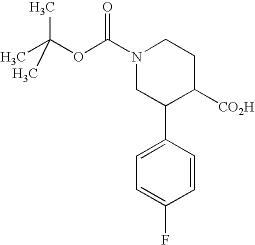 | 322 (M⁺ − 1) |
| 3(1) | 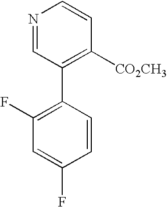 | 250 (M⁺ + 1) |
| 3(2) | 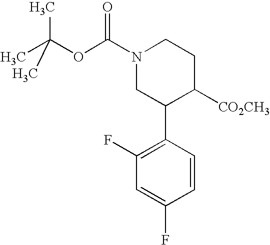 | 356 (M⁺ + 1) |
| 3(3) | 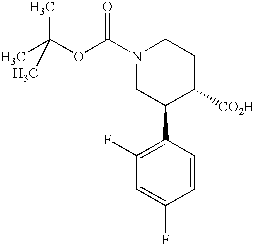 | 340 (M⁺ − 1) |
| | and | |
| | 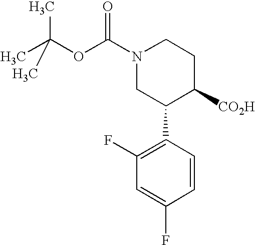 | |

TABLE 57

| Reference | | |
|---|---|---|
| Example No. | Structural formula | MS |
| 4(1) | 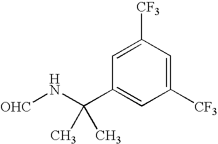 | 300 (M⁺ + 1) |
| 4(2) | 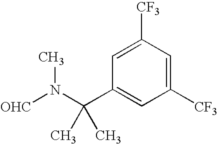 | 314 (M⁺ + 1) |
| 4(3) | 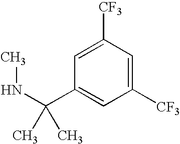 | 286 (M⁺ + 1) |
| 5 | 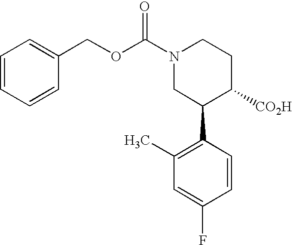 | 372 (M⁺ + 1) |
| 6 | 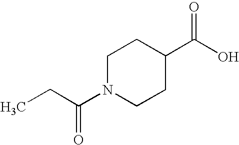 | 186 (M⁺ + 1) |
| 7 | 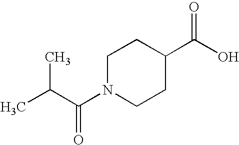 | 200 (M⁺ + 1) |
| 8 | 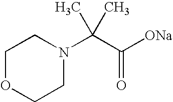 | 174 (M⁺ + 2-Na) |

TABLE 58

| Reference | | |
|---|---|---|
| Example | | |
| No. | Structural formula | MS |
| 9(1) | 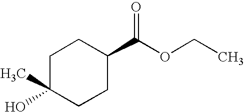 | 186 (M⁺ + 1) |
| 9(2) | 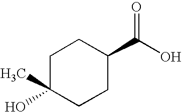 | 157 (M⁺ + 1) |
| 10 | 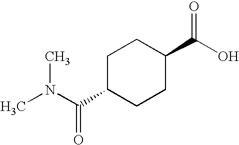 | 200 (M⁺ + 1) |
| 11 | 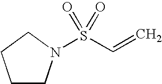 | 162 (M⁺ + 1) |
| 12(1) | 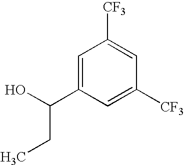 | 273 (M⁺ + 1) |
| 12(2) | 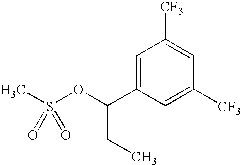 | 351 (M⁺ + 1) |
| 12(3) | 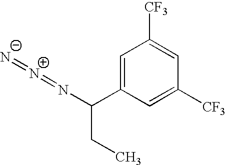 | 270 (M⁺ + 1 − N₂) |
| 12(4) | 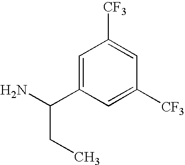 | 272 (M⁺ + 1) |

TABLE 59

| Reference | | |
|---|---|---|
| Example No. | Structural formula | MS |
| 13 | 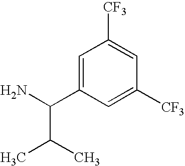 | 286 (M⁺ + 1) |

### INDUSTRIAL APPLICABILITY

The compound of the present invention or a salt thereof has an excellent tachykinin receptor antagonistic action. Further, the compound of the present invention or a salt thereof is excellent in terms of safety, absorption, penetration to the brain, metabolic stability, concentration in blood and sustainability, so that it has excellent pharmaceutical effects.

1. A piperidine compound represented by the formula [I]: 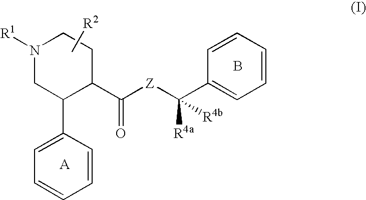
wherein
Ring A represents an optionally substituted benzene ring,
Ring B represents an optionally substituted benzene ring,
R¹ represents hydrogen atom, an optionally substituted amino group, an optionally substituted hydroxyl group, or substituted sulfinyl
R² represents hydrogen atom, an optionally substituted hydroxyl group, an optionally substituted amino group, an optionally substituted alkyl group, a substituted carbonyl group or a halogen atom,
Z represents oxygen atom or a group represented by the formula: —N(R³)—,
R³ represents hydrogen atom or an optionally substituted alkyl group,
R^{4a} and R^{4b} are the same or different from each other and each is hydrogen atom or an optionally substituted alkyl group, or may be bonded to each other at the both ends to form an alkylene group,
or a pharmaceutically acceptable salt thereof.

2. (canceled)

3. The compound or a pharmaceutically acceptable salt thereof according to claim 1, wherein
Ring A is a benzene ring represented by the formula: 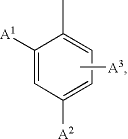
Ring B is a benzene ring represented by the formula: 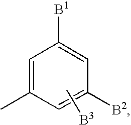
A¹ is hydrogen atom, a halogen atom or an alkyl group,
A² is a halogen atom or a halogen atom,
A³ is hydrogen atom,
B¹ is a trihalogenoalkyl group, an alkyl group, an alkoxy group or halogen atom,
B² is hydrogen atom, a trihalogenoalkyl group, an alkyl group or halogen atom,
B³ is hydrogen atom or a halogen atom,
R¹ is an alkylsulfinyl group; a
R² is hydrogen atom,
Z is a group represented by —N(R³)—, R³ is an alkyl group,
R^{4a} is hydrogen atom or an alkyl group,
R^{4b} is hydrogen atom or an alkyl group.

4. (canceled)

5. (canceled)

6. A process for preparing the piperidine compound represented by the formula [I]: 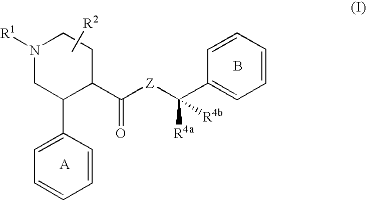
wherein
Ring A represents an optionally substituted benzene ring,
Ring B represents an optionally substituted benzene ring,
R¹ represents hydrogen atom, an optically substituted amino group, an optically substituted hydroxyl group, or a substituted sulfinyl group, R² represents hydrogen atom, an optionally substituted hydroxyl group, an optionally substituted amino group, an optionally substituted alkyl group, a substituted carbonyl group or a halogen atom,
Z represents oxygen atom or a group represented by the formula: —N(R³)—,
R³ represents hydrogen atom or an optionally substituted alkyl group,
R^{4a} and R^{4b} are the same or different from each other and each is hydrogen atom or an optionally substituted alkyl group, or may be bonded to each other at the both ends to form an alkylene group,
or a pharmaceutically acceptable salt thereof,
which comprises reacting a compound represented by the formula [II]: 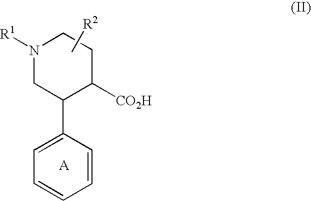
wherein Ring A, R¹ and R² have the same meanings as defined above,
with a compound represented by the formula [III]: 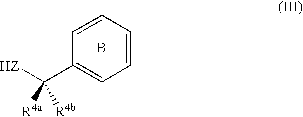
wherein Ring B, Z, R³, R^{4a} and R^{4b} have the same meanings as defined above,
in the presence of a condensing agent, and then, converting it into a pharmaceutically acceptable salt thereof, if necessary.

7. A pharmaceutical composition comprising the compound according to claim 1, in a clinically effective dose and a pharmaceutically acceptable carrier.

8. (canceled)

9. (canceled)

10. A method for treating and preventing a disease selected from inflammation, allergic diseases, pain, migraine, neuralgia, itchiness, cough, central nervous system disease, digestive organs disease nausea, emesis, urinary disorder, circulatory disease and immune disorder, comprising administering the compound according to claim 1 in a clinically effective dose to mammal.

11. The method according to claim 10, wherein the disease is urinary disorder.

12. A pharmaceutical composition comprising the compound according to claim 3, in a clinically effective dose and a pharmaceutically acceptable carrier.

13. A method for treating and preventing a disease selected from inflammation, allergic diseases, pain, migraine, neuralgia, itchiness, cough, central nervous system disease, digestive organs disease nausea, emesis, urinary disorder, circulatory disease and immune disorder, comprising administering the compound according to claim 3 in a clinically effective dose to mammal.

14. The method according to claim 13, wherein the disease is urinary disorder.
